SEQUENCE LISTING (91 sequences)

SEQ ID NO: 1 - protein, 206 aa, Artificial Sequence
  source: /note=“Description of Artificial Sequence Synthetic polypeptide”
  VARIANT (133)..(133): /replace=“Ile” or “Leu”
  SITE (1)..(206): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKNC

SEQ ID NO: 2 - protein, 205 aa, Artificial Sequence
  source: /note=“Description of Artificial Sequence Synthetic polypeptide”
  VARIANT (133)..(133): /replace=“Ile” or “Leu”
  SITE (1)..(205): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKN

SEQ ID NO: 3 - protein, 204 aa, Artificial Sequence
  source: /note=“Description of Artificial Sequence Synthetic polypeptide”
  VARIANT (133)..(133): /replace=“Ile” or “Leu”
  SITE (1)..(204): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFK

SEQ ID NO: 4 - protein, 203 aa, Artificial Sequence
  source: /note=“Description of Artificial Sequence Synthetic polypeptide”
  VARIANT (133)..(133): /replace=“Ile” or “Leu”
  SITE (1)..(203): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYF

SEQ ID NO: 5 - protein, 202 aa, Artificial Sequence
  source: /note=“Description of Artificial Sequence Synthetic polypeptide”
  VARIANT (133)..(133): /replace=“Ile” or “Leu”
  SITE (1)..(202): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEY

SEQ ID NO: 6 - protein, 201 aa, Artificial Sequence
  source: /note=“Description of Artificial Sequence Synthetic polypeptide”
  VARIANT (133)..(133): /replace=“Ile” or “Leu”
  SITE (1)..(201): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPE

SEQ ID NO: 7 - protein, 200 aa, Artificial Sequence
  source: /note=“Description of Artificial Sequence Synthetic polypeptide”
  VARIANT (133)..(133): /replace=“Ile” or “Leu”
  SITE (1)..(200): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHP

SEQ ID NO: 8 - protein, 199 aa, Artificial Sequence
  source: /note=“Description of Artificial Sequence Synthetic polypeptide”
  VARIANT (133)..(133): /replace=“Ile” or “Leu”
  SITE (1)..(199): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELH

SEQ ID NO: 9 - protein, 198 aa, Artificial Sequence
  source: /note=“Description of Artificial Sequence Synthetic polypeptide”
  VARIANT (133)..(133): /replace=“Ile” or “Leu”
  SITE (1)..(198): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVAREL

SEQ ID NO: 10 - protein, 197 aa, Artificial Sequence
  source: /note=“Description of Artificial Sequence Synthetic polypeptide”
  VARIANT (133)..(133): /replace=“Ile” or “Leu”
  SITE (1)..(197): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARE

SEQ ID NO: 11 - protein, 196 aa, Artificial Sequence
  source: /note=“Description of Artificial Sequence Synthetic polypeptide”
  VARIANT (133)..(133): /replace=“Ile” or “Leu”
  SITE (1)..(196): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVAR

SEQ ID NO: 12 - protein, 195 aa, Artificial Sequence
  source: /note=“Description of Artificial Sequence Synthetic polypeptide”
  VARIANT (133)..(133): /replace=“Ile” or “Leu”
  SITE (1)..(195): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVA

SEQ ID NO: 13 - protein, 194 aa, Artificial Sequence
  source: /note=“Description of Artificial Sequence Synthetic polypeptide”
  VARIANT (133)..(133): /replace=“Ile” or “Leu”
  SITE (1)..(194): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHV

SEQ ID NO: 14 - protein, 193 aa, Artificial Sequence
  source: /note=“Description of Artificial Sequence Synthetic polypeptide”
  VARIANT (133)..(133): /replace=“Ile” or “Leu”
  SITE (1)..(193): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHH

SEQ ID NO: 15 - protein, 192 aa, Artificial Sequence
  source: /note=“Description of Artificial Sequence Synthetic polypeptide”
  VARIANT (133)..(133): /replace=“Ile” or “Leu”
  SITE (1)..(192): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFH

SEQ ID NO: 16 - protein, 191 aa, Artificial Sequence
  source: /note=“Description of Artificial Sequence Synthetic polypeptide”
  VARIANT (133)..(133): /replace=“Ile” or “Leu”
  SITE (1)..(191): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAF

SEQ ID NO: 17 - protein, 190 aa, Artificial Sequence
  source: /note=“Description of Artificial Sequence Synthetic polypeptide”
  VARIANT (133)..(133): /replace=“Ile” or “Leu”
  SITE (1)..(190): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLA

SEQ ID NO: 18 - protein, 189 aa, Artificial Sequence
  source: /note=“Description of Artificial Sequence Synthetic polypeptide”
  VARIANT (133)..(133): /replace=“Ile” or “Leu”
  SITE (1)..(189): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRL

SEQ ID NO: 19 - protein, 188 aa, Artificial Sequence
  source: /note=“Description of Artificial Sequence Synthetic polypeptide”
  VARIANT (133)..(133): /replace=“Ile” or “Leu”
  SITE (1)..(188): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSR

SEQ ID NO: 20 - protein, 187 aa, Artificial Sequence
  source: /note=“Description of Artificial Sequence Synthetic polypeptide”
  VARIANT (133)..(133): /replace=“Ile” or “Leu”
  SITE (1)..(187): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDS

SEQ ID NO: 21 - protein, 186 aa, Artificial Sequence
  source: /note=“Description of Artificial Sequence Synthetic polypeptide”
  VARIANT (133)..(133): /replace=“Ile” or “Leu”
  SITE (1)..(186): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFD

SEQ ID NO: 22 - protein, 185 aa, Artificial Sequence
  source: /note=“Description of Artificial Sequence Synthetic polypeptide”
  VARIANT (133)..(133): /replace=“Ile” or “Leu”
  SITE (1)..(185): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRF

SEQ ID NO: 23 - protein, 184 aa, Artificial Sequence
  source: /note=“Description of Artificial Sequence Synthetic polypeptide”
  VARIANT (133)..(133): /replace=“Ile” or “Leu”
  SITE (1)..(184): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWR

SEQ ID NO: 24 - protein, 183 aa, Artificial Sequence
  source: /note=“Description of Artificial Sequence Synthetic polypeptide”
  VARIANT (133)..(133): /replace=“Ile” or “Leu”
  SITE (1)..(183): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEW

SEQ ID NO: 25 - protein, 182 aa, Artificial Sequence
  source: /note=“Description of Artificial Sequence Synthetic polypeptide”
  VARIANT (133)..(133): /replace=“Ile” or “Leu”
  SITE (1)..(182): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLE

SEQ ID NO: 26 - protein, 181 aa, Artificial Sequence
  source: /note=“Description of Artificial Sequence Synthetic polypeptide”
  VARIANT (133)..(133): /replace=“Ile” or “Leu”
  SITE (1)..(181): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVL

SEQ ID NO: 27 - protein, 180 aa, Artificial Sequence
  source: /note=“Description of Artificial Sequence Synthetic polypeptide”
  VARIANT (133)..(133): /replace=“Ile” or “Leu”
  SITE (1)..(180): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREV

SEQ ID NO: 28 - protein, 179 aa, Artificial Sequence
  source: /note=“Description of Artificial Sequence Synthetic polypeptide”
  VARIANT (133)..(133): /replace=“Ile” or “Leu”
  SITE (1)..(179): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGRE

SEQ ID NO: 29 - protein, 178 aa, Artificial Sequence
  source: /note=“Description of Artificial Sequence Synthetic polypeptide”
  VARIANT (133)..(133): /replace=“Ile” or “Leu”
  SITE (1)..(178): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGR

SEQ ID NO: 30 - protein, 177 aa, Artificial Sequence
  source: /note=“Description of Artificial Sequence Synthetic polypeptide”
  VARIANT (133)..(133): /replace=“Ile” or “Leu”
  SITE (1)..(177): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPG

SEQ ID NO: 31 - DNA, 621 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(621): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgctga

SEQ ID NO: 32 - DNA, 531 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(531): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccgggg

SEQ ID NO: 33 - protein, 158 aa, Artificial Sequence
  source: /note=“Description of Artificial Sequence Synthetic polypeptide”
MHQKRTAMFQDPQERPRKLPQLCTELQTTIHDIILECVYCKQQLLRREVYDFAFRDLCIVYRDGSPYAVCDKCLKFYSKISEYRHYCYSLYGTTLEQQYNKPLCDLLIRCINCQKPLCPEEKQRHLDKKQRFHNIRVRWTGRCMSCCRSSRTRRETQL

SEQ ID NO: 34 - DNA, 477 bp, Artificial Sequence
  source: /note=“Description of Artificial Sequence Synthetic polynucleotide”
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataagggtgcggtggaccggtcgatgtatgtcttgttgcagatcatcaagaacacgtagagaaacccagctgtaa

SEQ ID NO: 35 - DNA, 477 bp, Artificial Sequence
  source: /note=“Description of Artificial Sequence Synthetic polynucleotide”
atgcaccagaagcgcaccgccatgttccaggacccccaggagcgcccccgcaagctgccccagctgtgcaccgagctgcagaccaccatccacgacatcatcctggagtgcgtgtactgcaagcagcagctgctgcgccgcgaggtgtacgacttcgccttccgcgacctgtgcatcgtgtaccgcgacggcagcccctacgccgtgtgcgacaagtgcctgaagttctactccaagatcagcgagtaccgccactactgctacagcctgtacggcaccaccctggagcagcagtacaacaagcccctgtgcgacctgctgatccgctgcatcaactgccagaagcccctgtgccccgaggagaagcagcgccacctggacaagaagcagcgcttccacaacatccgggtgcggtggaccgggcgctgcatgagctgctgccgcagcagccgcacccgccgcgagacccagctgtaa

SEQ ID NO: 36 - protein, 98 aa, Artificial Sequence
  source: /note=“Description of Artificial Sequence Synthetic polypeptide”
MHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKP

SEQ ID NO: 37 - DNA, 297 bp, Artificial Sequence
  source: /note=“Description of Artificial Sequence Synthetic polynucleotide”
atgcatggagatacacctacattgcatgaatatatgttagatttgcaaccagagacaactggcctctacggctatggccaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 38 - DNA, 297 bp, Artificial Sequence
  source: /note=“Description of Artificial Sequence Synthetic polynucleotide”
atgcacggcgacacccccaccttgcacgagtacatgttggacttgcagcccgagaccaccggcctgtacggctacggccagttgaacgacagctccgaggaggaggacgagatcgacggccccgccggccaggccgagcccgaccgcgcccactacaacatcgtgaccttctgctgcaagtgcgactccaccctgcgcctgtgcgtgcagagcacccacgtggacatccgcaccttggaggacctgctgatgggcaccctgggcatcgtgtgccccatctgcagccagaagccctaa

SEQ ID NO: 39 - protein, 367 aa, Artificial Sequence
  source: /note=“Description of Artificial Sequence Synthetic polypeptide”
  VARIANT (133)..(133): /replace=“Ile” or “Leu”
  SITE (1)..(367): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKNCGPGPHQKRTAMFQDPQERPRKLPQLCTELQTTIHDIILECVYCKQQLLRREVYDFAFRDLCIVYRDGSPYAVCDKCLKFYSKISEYRHYCYSLYGTTLEQQYNKPLCDLLIRCINCQKPLCPEEKQRHLDKKQRFHNIRVRWTGRCMSCCRSSRTRRETQL

SEQ ID NO: 40 - DNA, 1104 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(1104): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccccaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataagggtgcggtggaccggtcgatgtatgtcttgttgcagatcatcaagaacacgtagagaaacccagctgtaa

SEQ ID NO: 41 - DNA, 1104 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(399): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccccaccagaagcgcaccgccatgttccaggacccccaggagcgcccccgcaagctgccccagctgtgcaccgagctgcagaccaccatccacgacatcatcctggagtgcgtgtactgcaagcagcagctgctgcgccgcgaggtgtacgacttcgccttccgcgacctgtgcatcgtgtaccgcgacggcagcccctacgccgtgtgcgacaagtgcctgaagttctactccaagatcagcgagtaccgccactactgctacagcctgtacggcaccaccctggagcagcagtacaacaagcccctgtgcgacctgctgatccgctgcatcaactgccagaagcccctgtgccccgaggagaagcagcgccacctggacaagaagcagcgcttccacaacatccgggtgcggtggaccgggcgctgcatgagctgctgccgcagcagccgcacccgccgcgagacccagctgtaa

SEQ ID NO: 42 - protein, 307 aa, Artificial Sequence
  source: /note=“Description of Artificial Sequence Synthetic polypeptide”
  VARIANT (133)..(133): /replace=“Ile” or “Leu”
  SITE (1)..(307): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKNCGPGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKP

SEQ ID NO: 43 - DNA, 924 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(924): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccccatggagatacacctacattgcatgaatatatgttagatttgcaaccagagacaactggcctctacggctatggccaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 44 - DNA, 924 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(924): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccccacggcgacacccccaccttgcacgagtacatgttggacttgcagcccgagaccaccggcctgtacggctacggccagttgaacgacagctccgaggaggaggacgagatcgacggccccgccggccaggccgagcccgaccgcgcccactacaacatcgtgaccttctgctgcaagtgcgactccaccctgcgcctgtgcgtgcagagcacccacgtggacatccgcaccttggaggacctgctgatgggcaccctgggcatcgtgtgccccatctgcagccagaagccctaa

SEQ ID NO: 45 - protein, 338 aa, Artificial Sequence
  source: /note=“Description of Artificial Sequence Synthetic polypeptide”
  VARIANT (133)..(133): /replace=“Ile” or “Leu”
  SITE (1)..(338): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGGPGPHQKRTAMFQDPQERPRKLPQLCTELQTTIHDIILECVYCKQQLLRREVYDFAFRDLCIVYRDGSPYAVCDKCLKFYSKISEYRHYCYSLYGTTLEQQYNKPLCDLLIRCINCQKPLCPEEKQRHLDKKQRFHNIRVRWTGRCMSCCRSSRTRRETQL

SEQ ID NO: 46 - DNA, 1017 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(1017): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggggacctgggccccaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataagggtgcggtggaccggtcgatgtatgtcttgttgcagatcatcaagaacacgtagagaaacccagctgtaa

SEQ ID NO: 47 - DNA, 1017 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(1017): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggggacctgggccccaccagaagcgcaccgccatgttccaggacccccaggagcgcccccgcaagctgccccagctgtgcaccgagctgcagaccaccatccacgacatcatcctggagtgcgtgtactgcaagcagcagctgctgcgccgcgaggtgtacgacttcgccttccgcgacctgtgcatcgtgtaccgcgacggcagcccctacgccgtgtgcgacaagtgcctgaagttctactccaagatcagcgagtaccgccactactgctacagcctgtacggcaccaccctggagcagcagtacaacaagcccctgtgcgacctgctgatccgctgcatcaactgccagaagcccctgtgccccgaggagaagcagcgccacctggacaagaagcagcgcttccacaacatccgggtgcggtggaccgggcgctgcatgagctgctgccgcagcagccgcacccgccgcgagacccagctgtaa

SEQ ID NO: 48 - protein, 278 aa, Artificial Sequence
  source: /note=“Description of Artificial Sequence Synthetic polypeptide”
  VARIANT (133)..(133): /replace=“Ile” or “Leu”
  SITE (1)..(278): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGGPGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKP

SEQ ID NO: 49 - DNA, 837 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(837): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggggacctgggccccatggagatacacctacattgcatgaatatatgttagatttgcaaccagagacaactggcctctacggctatggccaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 50 - DNA, 837 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(837): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggggacctgggccccacggcgacacccccaccttgcacgagtacatgttggacttgcagcccgagaccaccggcctgtacggctacggccagttgaacgacagctccgaggaggaggacgagatcgacggccccgccggccaggccgagcccgaccgcgcccactacaacatcgtgaccttctgctgcaagtgcgactccaccctgcgcctgtgcgtgcagagcacccacgtggacatccgcaccttggaggacctgctgatgggcaccctgggcatcgtgtgccccatctgcagccagaagccctaa

SEQ ID NO: 51 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 52 - protein, 40 aa, Mycobacterium tuberculosis
MTDVSRKIRAWGRRLMIGTAAAVVLPGLVGLAGGAATAGA

SEQ ID NO: 53 - protein, 285 aa, Mycobacterium tuberculosis
FSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 54 - protein, 495 aa, Artificial Sequence
  source: /note=“Description of Artificial Sequence Synthetic polypeptide”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGIRYPLTFGWCYKLVPVEPEKLEEANKGENTSLLHPVSLHGMDDPGREVLEWRFDSRLAFHHVARELHPEYFKNCGPGPFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 55 - protein, 4 aa, Artificial Sequence
  source: /note=“Description of Artificial Sequence Synthetic peptide”
GPGP

SEQ ID NO: 56 - DNA, 24 bp, Artificial Sequence
  source: /note=“Description of Artificial Sequence Synthetic primer”
tatagggccccatggagatacacc

SEQ ID NO: 57 - DNA, 27 bp, Artificial Sequence
  source: /note=“Description of Artificial Sequence Synthetic primer”
tatcgtcgacttatggttctgggaaca

SEQ ID NO: 58 - DNA, 18 bp, Artificial Sequence
  source: /note=“Description of Artificial Sequence Synthetic oligonucleotide”
caccatcaccatcaccat

SEQ ID NO: 59 - DNA, 20 bp, Artificial Sequence
  source: /note=“Description of Artificial Sequence Synthetic primer”
aatgtttcaggacccacagg

SEQ ID NO: 60 - DNA, 20 bp, Artificial Sequence
  source: /note=“Description of Artificial Sequence Synthetic primer”
ttgtttgcagctctgtgcat

SEQ ID NO: 61 - DNA, 22 bp, Artificial Sequence
  source: /note=“Description of Artificial Sequence Synthetic primer”
caagtgtgactctacgcttcgg

SEQ ID NO: 62 - DNA, 24 bp, Artificial Sequence
  source: /note=“Description of Artificial Sequence Synthetic primer”
gtggcccattaacaggtcttccaa

SEQ ID NO: 63 - protein, 16 aa, Artificial Sequence
  source: /note=“Description of Artificial Sequence Synthetic peptide”
CIVYRDGNPYAVCDKC

SEQ ID NO: 64 - protein, 15 aa, Artificial Sequence
  source: /note=“Description of Artificial Sequence Synthetic peptide”
EQQYNKPLCDLLIRC

SEQ ID NO: 65 - protein, 9 aa, Artificial Sequence
  source: /note=“Description of Artificial Sequence Synthetic peptide”
KLPQLCTEL

SEQ ID NO: 66 - protein, 8 aa, Artificial Sequence
  source: /note=“Description of Artificial Sequence Synthetic peptide”
YDFAFRDL

SEQ ID NO: 67 - protein, 9 aa, Artificial Sequence
  source: /note=“Description of Artificial Sequence Synthetic peptide”
RCINCQKPL

SEQ ID NO: 68 - protein, 9 aa, Artificial Sequence
  source: /note=“Description of Artificial Sequence Synthetic peptide”
DKKQRFMNI

SEQ ID NO: 69 - protein, 17 aa, Artificial Sequence
  source: /note=“Description of Artificial Sequence Synthetic peptide”
QAEPDRAHYNIVTFCCK

SEQ ID NO: 70 - protein, 8 aa, Artificial Sequence
  source: /note=“Description of Artificial Sequence Synthetic peptide”
DLYCYEQL

SEQ ID NO: 71 - protein, 9 aa, Artificial Sequence
  source: /note=“Description of Artificial Sequence Synthetic peptide”
RAHYNIVTF

SEQ ID NO: 72 - protein, 9 aa, Artificial Sequence
  source: /note=“Description of Artificial Sequence Synthetic peptide”
LCVQSTHVD

SEQ ID NO: 73 - protein, 8 aa, Artificial Sequence
  source: /note=“Description of Artificial Sequence Synthetic peptide”
ITQMYTNV

SEQ ID NO: 74 - DNA, 1488 bp, Artificial Sequence
  source: /note=“Description of Artificial Sequence Synthetic polynucleotide”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccctttagcaggcctggcctgcccgtggaatacctgcaagtgccttcccctagcatgggaagagatattaaggtgcagttccagagcggcggaaacaacagccccgctgtttacctgctcgacggcctgagagcccaggatgactacaacggctgggacatcaacacccctgccttcgagtggtactaccagtctggcctgtctatcgtgatgccagtgggcggccagagcagcttctacagcgactggtatagccctgcctgcggcaaggccggttgtcagacctacaaatgggagacattcctgaccagcgagctgcctcagtggctgtccgccaatcgggcggtcaagccaaccggcagcgccgctatcggcctgagcatggccggcagcagcgccatgatcctggccgcttatcacccccaacaatttatctacgccggctccctgagcgccctgctggaccccagccagggcatgggacctagcctgatcggacttgctatgggcgatgctggaggctacaaggccgccgacatgtggggaccttcttctgatcctgcctgggagagaaacgaccctacacagcagatccccaagctggtggccaacaataccagactgtgggtgtactgcggcaacggaacacctaacgagctgggcggagccaacatccctgccgagttcctggaaaacttcgtgcggagctctaatctgaagttccaggacgcctacaatgccgccggcggccacaacgccgtgttcaacttcccacctaacggcacccacagctgggaatactggggcgctcagctgaacgccatgaaaggcgacctgcagtcctctctgggagccggatga

SEQ ID NO: 75 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 76 - protein, 33 aa, Mycobacterium tuberculosis
IEAAASAIQGNVTSIHSLLDEGKQSLTKLAAAW

SEQ ID NO: 77 - protein, 37 aa, Mycobacterium tuberculosis
EAYQGVQQKWDATATELNNALQNLARTISEAGQAMAS

SEQ ID NO: 78 - protein, 304 aa, Artificial Sequence
  source: /note=“Description of Artificial Sequence Synthetic polypeptide”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGIRYPLTFGWCYKLVPVEPEKLEEANKGENTSLLHPVSLHGMDDPGREVLEWRFDSRLAFHHVARELHPEYFKNCGPGPTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 79 - DNA, 915 bp, Artificial Sequence
  source: /note=“Description of Artificial Sequence Synthetic polynucleotide”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggcccaccgagcagcaatggaacttcgccggcatcgaggccgctgcttccgccatccagggcaacgtgaccagcatccacagcctgctggacgagggcaagcagagcctgaccaagctggccgccgcttggggcggatctggctctgaagcctaccagggcgtgcagcagaaatgggatgccacagccacagaactgaacaacgccctgcagaacctggctagaaccatcagcgaggccggacaggccatggccagcaccgaaggcaatgtgacaggcatgttcgcctga

SEQ ID NO: 80 - DNA, 35 bp, Artificial Sequence
  source: /note=“Description of Artificial Sequence Synthetic primer”
taatacgactcactatagggcgccaccatggtgag

SEQ ID NO: 81 - DNA, 25 bp, Artificial Sequence
  source: /note=“Description of Artificial Sequence Synthetic primer”
tgcagtgaaaaaaatgctttatttg

SEQ ID NO: 82 - DNA, 36 bp, Artificial Sequence
  source: /note=“Description of Artificial Sequence Synthetic primer”
taatacgactcactatagggcgccaccatgcacggc

SEQ ID NO: 83 - DNA, 21 bp, Artificial Sequence
  source: /note=“Description of Artificial Sequence Synthetic primer”
gtcgacttagggcttctggct

SEQ ID NO: 84 - DNA, 25 bp, Artificial Sequence
  source: /note=“Description of Artificial Sequence Synthetic primer”
tgacacctactcagacaatgcgatg

SEQ ID NO: 85 - protein, 495 aa, Artificial Sequence
  source: /note=“Description of Artificial Sequence Synthetic polypeptide”
  VARIANT (133)..(133): /replace=“Ile” or “Leu”
  SITE (1)..(495): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKNCGPGPFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 86 - DNA, 1488 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(1488): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggccctttagcaggcctggcctgcccgtggaatacctgcaagtgccttcccctagcatgggaagagatattaaggtgcagttccagagcggcggaaacaacagccccgctgtttacctgctcgacggcctgagagcccaggatgactacaacggctgggacatcaacacccctgccttcgagtggtactaccagtctggcctgtctatcgtgatgccagtgggcggccagagcagcttctacagcgactggtatagccctgcctgcggcaaggccggttgtcagacctacaaatgggagacattcctgaccagcgagctgcctcagtggctgtccgccaatcgggcggtcaagccaaccggcagcgccgctatcggcctgagcatggccggcagcagcgccatgatcctggccgcttatcacccccaacaatttatctacgccggctccctgagcgccctgctggaccccagccagggcatgggacctagcctgatcggacttgctatgggcgatgctggaggctacaaggccgccgacatgtggggaccttcttctgatcctgcctgggagagaaacgaccctacacagcagatccccaagctggtggccaacaataccagactgtgggtgtactgcggcaacggaacacctaacgagctgggcggagccaacatccctgccgagttcctggaaaacttcgtgcggagctctaatctgaagttccaggacgcctacaatgccgccggcggccacaacgccgtgttcaacttcccacctaacggcacccacagctgggaatactggggcgctcagctgaacgccatgaaaggcgacctgcagtcctctctgggagccggatga

SEQ ID NO: 87 - protein, 304 aa, Artificial Sequence
  source: /note=“Description of Artificial Sequence Synthetic polypeptide”
  VARIANT (133)..(133): /replace=“Ile” or “Leu”
  SITE (1)..(304): /note=“Variant residues given in the sequence have no preference with respect to those in the annotations for variant positions”
MGCKWSKSSVVGWPAVRERMRRAEPAADGVGAASRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPEKLEEANKGENTSLLHPVSLHGMDDPGREVLEWRFDSRLAFHHVARELHPEYFKNCGPGPTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 88 - DNA, 915 bp, Artificial Sequence
  source: /note=“Description of Artificial Sequence Synthetic polynucleotide”
  variation (397)..(399): /replace=“atc” or “ata” or “att” or “cta” or “ctc” or “ctg” or “ctt” or “gta” or “gtc” or “gtg” or “tta” or “ttg”
  misc_feature (1)..(915): /note=“Variant nucleotides given in the sequence have no preference with respect to those in the annotations for variant positions”
atgggttgcaagtggtcaaaaagtagtgtggttggatggcctgctgtaagggaaagaatgagacgagctgagccagcagcagatggggtgggagcagcatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccaggaccaggggttagatatccactgacctttggatggtgctacaagctagtaccagttgagccagagaagttagaagaagccaacaaaggagagaacaccagcttgttacaccctgtgagcctgcatggaatggatgacccggggagagaagtgttagagtggaggtttgacagccgcctagcatttcatcacgtggcccgagagctgcatccggagtacttcaagaactgcggacctgggcccaccgagcagcaatggaacttcgccggcatcgaggccgctgcttccgccatccagggcaacgtgaccagcatccacagcctgctggacgagggcaagcagagcctgaccaagctggccgccgcttggggcggatctggctctgaagcctaccagggcgtgcagcagaaatgggatgccacagccacagaactgaacaacgccctgcagaacctggctagaaccatcagcgaggccggacaggccatggccagcaccgaaggcaatgtgacaggcatgttcgcctga

SEQ ID NO: 89 - protein, 6 aa, Artificial Sequence
  source: /note=“Description of Artificial Sequence Synthetic 6xHis tag”
HHHHHH

SEQ ID NO: 90 - DNA, 80 bp, Artificial Sequence
  source: /note=“Description of Artificial Sequence Synthetic oligonucleotide”
tacttcaagaactgcggacctgggcccggccaatctttcccgggggtaccgtcgactgcggccgcgaattccaagctttg

SEQ ID NO: 91 - DNA, 30 bp, Artificial Sequence
  source: /note=“Description of Artificial Sequence Synthetic oligonucleotide”
taatacgactcactatagggcgccaccatg

(19) United States
(12) Patent Application Publication
(10) Pub. No.: US 2023/0173063 A1
(43) Pub. Date: Jun. 8, 2023

(54) EXOSOME-ANCHORING FUSION PROTEINS AND VACCINES

(71) Applicant: BiovelocITA S.r.l., Milan (IT)

(72) Inventors: Cristina PELIZON, Milan (IT); Maurizio Paolo Maria FEDERICO, Rome (IT)

(21) Appl. No.: 17/923,210

(22) PCT Filed: May 4, 2021
(86) PCT No.: PCT/EP2021/061730

§ 371 Date: Nov. 3, 2022

(30) Foreign Application Priority Data
May 4, 2020 (IT) 102020000009688

(51) Int. Cl. A61K 39/385; A61K 39/00; A61K 39/12

(52) U.S. Cl. CPC A61K 39/385; A61K 39/0011; A61K 39/12; A61K 2039/53; A61K 2039/55555; A61K 2039/572; A61K 2039/585; A61K 2039/6075; A61K 2039/70

(57) ABSTRACT

Provided herein are exosome-anchoring fusion proteins and methods of using the exosome-anchoring fusion proteins for immunization to treat or prevent diseases such as virus infections or cancer.

### 1. CROSS-REFERENCE TO RELATED APPLICATIONS

This application claims the right of priority based on Italian application serial no. 102020000009688, filed May 4, 2020, the disclosure of which is incorporated herein by reference in its entirety.

### 2. BACKGROUND

Vaccines are effective tools for preventing and treating viral infections, bacterial infections, and toxicities caused by bacterial proteins. Vaccines have also been shown to be effective in preventing and treating various types of cancer caused by viral infection. More recently, vaccines have been proposed for directly treating cancers by stimulating immune responses to tumor neoantigens or to oncofetal antigens having restricted expression in normal adult tissues.

B cells play a critical role in adaptive immunity stimulated by vaccines, providing protection from pathogen through the production of specific antibodies. The activation and activity of T cells, including CD4⁺ and CD8⁺ T cells, is also an important part of the adaptive immune response to vaccination. Vaccines designed to induce T cells can directly contribute to pathogen clearance via cell-mediated mechanisms and can directly kill tumor cells expressing the targeted tumor antigen.

Exosomes are membrane-bound extracellular vesicles produced by inward budding of late endosomes. Exosomes have biological activities in vivo and exert significant roles in various pathological conditions such as cancer, autoimmune diseases, infectious and neurodegenerative diseases. Dendritic cell-derived exosomes express MHC I, MHC II, and costimulatory molecules and have been proven to be able to induce and enhance antigen-specific T cell responses in vivo. Clinical trials have demonstrated the feasibility of exosomes as cell-free vaccines in patients. However, the therapeutic efficacy of exosome vaccines appears to be limited, and cell-free exosome vaccine has been approved for use.

There is, therefore, a need in the art for approaches that increase the immunogenicity of exosome vaccines.

### 3. SUMMARY

We have designed and tested fusion proteins that comprise an exosome-anchoring polypeptide domain and an immunogenic antigen polypeptide domain. The fusion proteins can be used for treating or preventing diseases such as virus infections, bacterial infections, and cancer. We have also demonstrated that both plasmid expression vectors encoding the fusion protein and RNA transcripts encoding the fusion protein are effective vehicles for introducing the fusion proteins into cells for packaging into exosomes.

Accordingly, in a first aspect, a fusion protein is provided herein. The fusion protein comprises from N-terminus to C-terminus, (a) an exosome-anchoring polypeptide domain and (b) an immunogenic antigen polypeptide domain, wherein the exosome-anchoring polypeptide is a truncated Nef^{mut} protein having an amino acid sequence selected from SEQ ID NOs: 1-30.

In some embodiments, the exosome-anchoring polypeptide has the amino acid sequence of SEQ ID NO: 1. In some embodiments, the exosome-anchoring polypeptide has the amino acid sequence of SEQ ID NO: 30.

In some embodiments, the immunogenic antigen is a virus antigen or a tumor antigen.

In some embodiments, the immunogenic antigen is a virus antigen. In some embodiments, the virus antigen is selected from the group consisting of: a human papillomavirus (HPV) antigen, a human immunodeficiency virus (HIV) antigen, a hepatitis B virus (HBV) antigen, a hepatitis C virus (HCV) antigen, an Ebola virus antigen, a West Nile virus antigen, a Crimean-Congo virus antigen, a dengue virus antigen, and an influenza virus antigen.

In some embodiments, the virus antigen is a human papillomavirus (HPV) antigen. In some embodiments, the HPV antigen is E6 or E7 of human papillomavirus.

In some embodiments, the virus antigen is a human immunodeficiency virus (HIV) antigen. In some embodiments, the HIV antigen is Gag or Tat of human immunodeficiency virus.

In some embodiments, the virus antigen is a hepatitis B virus (HBV) antigen. In some embodiments, the HBV antigen is Core of hepatitis B virus.

In some embodiments, the virus antigen is a hepatitis C virus (HCV) antigen. In some embodiments, the HCV antigen is Core, NS3, E1, or E2 of hepatitis C virus.

In some embodiments, the virus antigen is an Ebola virus antigen. In some embodiments, the Ebola virus antigen is VP24, VP40, NP, or GP of Ebola virus.

In some embodiments, the virus antigen is a West Nile virus antigen. In some embodiments, the West Nile virus antigen is NS3 of West Nile virus.

In some embodiments, the virus antigen is a Crimean-Congo virus antigen. In some embodiments, the Crimean-Congo virus antigen is GP or NP of Crimean-Congo virus.

In some embodiments, the virus antigen is a dengue virus antigen.

In some embodiments, the virus antigen is an influenza virus antigen. In some embodiments, the influenza virus is selected from the group consisting of: parainfluenza virus 1, parainfluenza virus 2, influenza A virus, and influenza B virus. In some embodiments, the influenza virus is influenza A virus. In some embodiments, the virus antigen is the nucleoprotein (NP) or the matrix protein (M1) of influenza A virus.

In some embodiments, the immunogenic antigen is a bacteria antigen. In some embodiments, the bacteria antigen is a Mycobacterium tuberculosis antigen. In some embodiments, the bacteria antigen is Ag85B or ESAT-6.

In some embodiments, the immunogenic antigen is a parasite antigen. In some embodiments, the parasite antigen is a Plasmodium antigen.

In some embodiments, the immunogenic antigen is a tumor antigen. In some embodiments, the tumor antigen is a tumor-specific antigen. In some embodiments, the tumor antigen is a tumor-associated antigen.

In another aspect, provided herein is a polynucleotide encoding the fusion protein described above.

In some embodiments, the polynucleotide is DNA.

In some embodiments, the polynucleotide is RNA. In some embodiments, the polynucleotide is messenger RNA (mRNA), e.g., mRNA suitable for translation obtained by T7 RNA polymerase transcription from a DNA template.

In another aspect, provided herein is a vector comprising at least one polynucleotide described above, wherein the vector expresses at least one fusion protein described above. In some embodiments, the vector is a plasmid vector. In some embodiments, the vector is a viral vector. In some embodiments, the viral vector is an adenovirus vector, an adeno-associated virus (AAV) vector, or a vaccinia vector.

In another aspect, provided herein is an extracellular vesicle comprising the fusion protein, the polynucleotide, or the vector described above. In some embodiments, the extracellular vesicle is an exosome.

In another aspect, provided herein is a nanoparticle comprising the fusion protein, the polynucleotide, or the vector described above.

In another aspect, provided herein is a vaccine composition comprising the fusion protein, the polynucleotide, the vector, the extracellular vesicle, or the nanoparticle described above, and a pharmaceutically acceptable excipient. In some embodiments, the vaccine composition is formulated for intramuscular administration.

In another aspect, provided herein is a method of treating or preventing a disease or condition in a subject in need thereof through immunization. The method comprises administering to the subject an effective amount of the fusion protein, the polynucleotide, the vector, the extracellular vesicle, the nanoparticle, or the pharmaceutical composition.

In some embodiments, a plurality of immunogenic antigens are administered to the subject. In some embodiments, the immunogenic antigens are expressed from the same vector. In some embodiments, the immunogenic antigens are expressed from different vectors. In some embodiments, the immunogenic antigens are administered simultaneously to the subject.

In some embodiments, the disease or condition is a viral infection. In some embodiments, the disease or condition is cancer.

In some embodiments, the fusion protein, the polynucleotide, the vector, the extracellular vesicle, the nanoparticle, or the pharmaceutical composition is administered by intramuscular administration. In some embodiments, the method further comprises electroporation immediately after the intramuscular administration.

In another aspect, provided herein is a method of inducing an antigen-specific cytotoxic T-lymphocyte (CTL) and/or antigen-specific CD4⁺ T cell response in a subject in need thereof. The method comprises administering to the subject an effective amount of the fusion protein, the polynucleotide, the vector, the extracellular vesicle, the nanoparticle, or the pharmaceutical composition.

In some embodiments, a plurality immunogenic antigens are administered to the subject. In some embodiments, the immunogenic antigens are expressed from the same vector. In some embodiments, the immunogenic antigens are expressed from different vectors. In some embodiments, the immunogenic antigens are administered simultaneously to the subject.

In some embodiments, the subject has or is at risk for a viral infection. In some embodiments, the subject has or is at risk for cancer. In some embodiments, the subject has or is at risk for a bacterial infection.

In some embodiments, the fusion protein, the polynucleotide, the vector, the extracellular vesicle, the nanoparticle, or the pharmaceutical composition is administered by intramuscular administration. In some embodiments, the method further comprises electroporation immediately after the intramuscular administration.

In another aspect, provided herein is a fusion protein, comprising: from N-terminus to C-terminus, (a) an exosome-anchoring polypeptide domain and (b) an immunogenic antigen polypeptide domain, wherein the exosome-anchoring polypeptide is a Nef^{mut} protein or a truncated Nef^{mut} protein having an amino acid sequence selected from SEQ ID NOs: 1-30, and wherein the immunogenic antigen has the amino acid sequence of SEQ ID NO: 33, SEQ ID NO: 36, SEQ ID NO: 51 or SEQ ID NO: 75.

In some embodiments, the exosome-anchoring polypeptide is a Nef^{mut} protein having the amino acid sequence of SEQ ID NO: 1, and wherein the immunogenic antigen has the amino acid sequence of SEQ ID NO: 33. In some embodiments, the fusion protein has the amino acid sequence of SEQ ID NO: 39.

In some embodiments, the exosome-anchoring polypeptide is a Nef^{mut} protein having the amino acid sequence of SEQ ID NO: 1, and wherein the immunogenic antigen has the amino acid sequence of SEQ ID NO: 36. In some embodiments, the fusion protein has the amino acid sequence of SEQ ID NO: 42.

In some embodiments, the exosome-anchoring polypeptide is a truncated Nef^{mut} protein having the amino acid sequence of SEQ ID NO: 30, and wherein the immunogenic antigen has the amino acid sequence of SEQ ID NO: 33. In some embodiments, the fusion protein has the amino acid sequence of SEQ ID NO: 45.

In some embodiments, the exosome-anchoring polypeptide is a truncated Nef^{mut} protein having the amino acid sequence of SEQ ID NO: 30, and wherein the immunogenic antigen has the amino acid sequence of SEQ ID NO: 36. In some embodiments, the fusion protein has the amino acid sequence of SEQ ID NO: 48.

In some embodiments, the exosome-anchoring polypeptide is a truncated Nef^{mut} protein having the amino acid sequence of SEQ ID NO: 30, and wherein the immunogenic antigen has the amino acid sequence of SEQ ID NO: 51. In some embodiments, the fusion protein has the amino acid sequence of, SEQ ID NO: 85.

In some embodiments, the exosome-anchoring polypeptide is a truncated Nef^{mut} protein having the amino acid sequence of SEQ ID NO: 30, and wherein the immunogenic antigen has the amino acid sequence of SEQ ID NO: 75. In some embodiments, the fusion protein has the amino acid sequence of Nef^{mut}+ESAT-6 fusion protein SEQ ID NO: 87.

In another aspect, provided herein is a polynucleotide encoding the fusion protein described above. In some embodiments, the polynucleotide is DNA. In some embodiments, the polynucleotide has the nucleotide sequence of SEQ ID NOs: 38, 41, 44, 47, 74, or 79.

In some embodiments, the polynucleotide is RNA.

In another aspect, provided herein is a vector comprising at least one polynucleotide described above, wherein the vector expresses at least one fusion protein described above. In some embodiments, the vector is a plasmid vector. In some embodiments, the vector is a viral vector.

In another aspect, provided herein is an extracellular vesicle comprising the fusion protein, the polynucleotide, or the vector described above. In some embodiments, the extracellular vesicle is an exosome.

In another aspect, provided herein is a nanoparticle comprising the fusion protein, the polynucleotide, or the vector described above.

In another aspect, provided herein is a pharmaceutical composition comprising the fusion protein, the polynucleotide, the vector, the extracellular vesicle, or the nanoparticle described above, and a pharmaceutically acceptable excipient. In some embodiments, the pharmaceutical composition is formulated for intramuscular administration.

In another aspect, provided herein is a method of treating or preventing a human papillomavirus (HPV) infection. The method comprises administering to a patient who has or is at risk for HPV infection an effective amount of the fusion protein, the polynucleotide, the vector, the extracellular vesicle, the nanoparticle, or the pharmaceutical composition.

In some embodiments, E6 and E7 antigens of HPV16 are administered to the patient. In some embodiments, the E6 and E7 antigens of HPV16 are expressed from the same vector or mRNA. In some embodiments, the E6 and E7 antigens of HPV16 are expressed from different vectors or mRNAs. In some embodiments, the E6 and E7 antigens of HPV16 are administered simultaneously to the patient.

In some embodiments, the fusion protein, the polynucleotide, the vector, the extracellular vesicle, the nanoparticle, or the pharmaceutical composition is administered by intramuscular administration. In some embodiments, the method further comprises electroporation immediately after the intramuscular administration.

In another aspect, provided herein is a method of inducing a cytotoxic T-lymphocyte (CTL) and/or antigen-specific CD4⁺ T cell response in a patient who has or is at risk for HPV infection. The method comprises administering to a patient who has or is at risk for HPV infection an effective amount of the fusion protein, the polynucleotide, the vector, the extracellular vesicle, the nanoparticle, or the pharmaceutical composition.

In some embodiments, E6 and E7 antigens of HPV16 are administered to the patient. In some embodiments, the E6 and E7 antigens of HPV16 are expressed from the same vector or mRNA. In some embodiments, the E6 and E7 antigens of HPV16 are expressed from different vectors or mRNAs. In some embodiments, the E6 and E7 antigens of HPV16 are administered simultaneously to the patient.

In some embodiments, the fusion protein, the polynucleotide, the vector, the extracellular vesicle, the nanoparticle, or the pharmaceutical composition is administered by intramuscular administration. In some embodiments, the method further comprises electroporation immediately after the intramuscular administration.

### 4. BRIEF DESCRIPTION OF THE DRAWINGS

These and other features, aspects, and advantages of the present invention will become better understood with regard to the following description, and accompanying drawings, where:

FIGS. 1A and 1B show the structure of the DNA molecular construct expressing Nef^{mut}/HPV16-E7, with FIG. 1A showing a schematic of the construct in which “ie-CMV” is the immediate-early CMV promoter; “SD” is the major splice donor site; “SA” is the major splice acceptor site; “polyA” is the polyadenylation site, and FIG. 1B showing a portion of the nucleotide sequence, including from 5′ to 3′ end, the 3′ end of Nef^{mut} ORF, the downstream GPGP (SEQ ID NO: 55) linker including the Apa I site, and the other relevant restriction sites of the pTarget-Nef^{mut} expression vector.

FIGS. 2A and 2B show HPV16 E7-specific CD8⁺ and CD4⁺ T cell response induced in mice injected with Nef^{mut}/E7 expressing DNA vector with or without subsequent electroporation (EP), with FIG. 2A showing HPV16 E7-specific CD8⁺ T cell response and FIG. 2B showing HPV16 E7-specific CD4⁺ T cell response. C57BL/6 mice were inoculated twice with either the Nef^{mut}/E7-DNA vector or the empty vector alone (Nil). At the time of sacrifice, 2.5×10⁵ splenocytes/well were incubated overnight with 5 μg/ml of unrelated (not shown) or E7-specific peptides in triplicate IFN-γ ELISpot microwells. The number of IFN-γ spot-forming units (SFU)/well are shown as mean values of triplicates. Intragroup mean values are also reported. ND: not done.

FIGS. 3A and 3B show the detection of Nef^{mut}/E6 based fusion products in transfected cells and respective exosomes, with FIG. 3A showing the fusion protein expression in transfected cells and FIG. 3B showing the fusion protein in exosomes. Western blot analysis was performed with 30 μg of cell lysates from 293T cells transfected with DNA vectors expressing the indicated Nef^{mut}-based fusion products, and equal volumes of buffer where purified exosomes were resuspended after differential centrifugations of the respective supernatants. As control, conditions from mock-transfected cells as well as cells transfected with wild type (wt) Nef were included. Polyclonal anti-Nef Abs served to detect Nef^{mut}-based products, while β-actin and Alix were markers for cell lysates and exosomes, respectively. The relevant protein products are indicated by arrows: for lanes 2, 3 and 4, the arrow on the left indicates the wt Nef protein, Nef^{mut} protein, and Nef^{mut}PL protein. Due to its shorter length, Nef^{mut}PL runs slightly faster on SDS gel. For lanes 5, 6 and 7, the arrows in the middle indicate Nef^{mut}/E6 based product and a shorter Nef^{mut}/E6 based degradation product. The arrow on the right indicates the Nef^{mut}PL/E6 based product (lanes 8 and 9). Positive CTRL: cells and exosomes from transfection with pcDNA3-Nef^{mut}/GFP. Molecular markers are provided in kDa.

FIGS. 4A and 4B show the detection of Nef^{mut}/E7 based fusion products in transfected cells and respective exosomes with FIG. 4A showing the fusion protein expression in transfected cells and FIG. 4B showing the fusion protein in exosomes. Western blot analysis was performed with 30 μg of cell lysates from 293T cells transfected with DNA vectors expressing the indicated Nef^{mut}-based fusion products, and equal volumes of buffer where purified exosomes were resuspended after differential centrifugations of the respective supernatants. As control, conditions from mock-transfected cells as well as cells transfected with Nef^{mut} alone were included. Polyclonal anti-Nef Abs served to detect Nef^{mut}-based products, while β-actin and Alix were markers for cell lysates and exosomes, respectively. Nef protein products are indicated by arrows: for lanes 2 and 3, the arrow on the left indicates Nef^{mut} protein and Nef^{mut}PL protein. Due to its shorter length, Nef^{mut}PL runs slightly faster on SDS gel. The arrow on the right indicates the Nef^{mut}/E7 based product and Nef^{mut}PL/E7 based product (lanes 4 to 9). Molecular markers are provided in kDa.

FIGS. 5A and 5B show the analysis of HPV16 E6 and E7 products in transfected cells and respective exosomes, with FIG. 5A showing the expression of HPV16 E6 and E7 products in transfected cells and FIG. 5B showing the HPV16 E6 and E7 products in exosomes. Western blot analysis from 30 μg of cell lysates from 293T cells transfected with DNA vectors expressing the indicated HPV16 ORFs, and equal volumes of buffer where purified exosomes were resuspended after differential centrifugations of the respective supernatants. As control, conditions from mock-transfected cells as well as cells transfected with Nef^{mut} fused with a scFv (Nef^{mut} GO) including a His tag at its C-terminus were included. Polyclonal anti-His-tag antibodies served to detect both HPV16-related (indicated by an arrow) and Nef^{mut}-GO products, while β-actin and Alix were detected as markers for cell lysates and exosomes, respectively. Relevant protein products are indicated by arrows. Molecular markers are provided in kDa.

FIGS. 6A and 6B show HPV16 E6-specific and E7-specific CD8⁺ and CD4⁺ T cell immunity induced in mice by injection (intramuscular+electroporation) (i.m.+EP) of the indicated DNA vectors, with FIG. 6A showing HPV16 E6-specific and E7-specific CD8⁺ T cell response and FIG. 6B showing HPV16 E6-specific and E7-specific CD4⁺ T cell response. C57BL/6 mice were inoculated (i.m.+EP) with 10 μg of the DNA vectors expressing Nef^{mut}-based products. As control, mice were inoculated with the empty vector (Nil). At the time of sacrifice, 2.5×10⁵ splenocytes were incubated overnight with 5 μg/ml of unrelated (not shown), E6-specific, or E7-specific peptides in triplicate IFN-γ ELISpot microwells. Shown are the number of IFN-γ spot-forming units (SFU)/well as mean values of triplicates. Intragroup mean values are also reported.

FIGS. 7A, 7B, and 7C show IFN-7, IL-2 and TNF-α intracellular accumulation in CD8⁺ T cells from cultures of splenocytes isolated from each mouse injected (i.m.+EP) with the indicated DNA vectors, with FIG. 7A showing IFN-γ intracellular accumulation in CD8⁺ T cells, FIG. 7B showing IL-2 intracellular accumulation in CD8⁺ T cells, and FIG. 7C showing TNF-α intracellular accumulation in CD8⁺ T cells. Cryopreserved splenocytes were incubated overnight with 5 μg/ml of unrelated (not shown), E6-specific, or E7-specific peptides in the presence of Brefeldin A, and then analyzed by intracellular cytokine staining (ICS). For each mouse, absolute percentages of positive CD8⁺ T cells within total alive CD8⁺ T cells for each cytokine, subtracted values detected in CD8⁺ T cells from cultures treated with unrelated peptide, are reported. In the histograms, each bar represents an individual animal.

FIGS. 8A, 8B, and 8C show intragroup mean values+standard deviation of the percentages of IFN-γ, IL-2 and TNF-α accumulating CD8⁺ T cells within total CD8⁺ T cells from cultures of splenocytes isolated from each mouse injected (i.m.+EP) with the indicated DNA vectors, with FIG. 8A showing intragroup mean values+SD of the percentages of IFN-γ accumulating CD8⁺ T cells, FIG. 8B showing intragroup mean values+SD of the percentages of IL-2 accumulating CD8⁺ T cells, and FIG. 8C showing intragroup mean values+SD of the percentages of TNF-α accumulating CD8⁺ T cells. Shown are mean values+SD of the absolute percentages of positive CD8⁺ T cells from cultures treated with specific peptides subtracted values detected in CD8⁺ T cells from cultures treated with unrelated peptide. In the histograms, each bar represents a group of animals.

FIGS. 9A and 9B show IFN-γ, IL-2 and TNF-α intracellular accumulation in CD8⁺ T cells from cultures of splenocytes isolated from each mouse injected (i.m.+EP) with the indicated DNA vectors as determined by detection of single, double, and triple cytokine positive CD8⁺ T cell sub-populations, with FIG. 9A showing the results from Nef^{mut}/E7, Nef^{mut}/E7^{OD}, and Nef^{mut}PL/E7^{OD} injected mice and FIG. 9B showing the results from Nef^{mut}/E6, Nef^{mut}/E6^{OD}, and Nef^{mut}PL/E6^{OD} injected mice. Cryopreserved splenocytes were incubated overnight with 5 μg/ml of unrelated (not shown), E6- or E7-specific peptides in the presence of Brefeldin A, and then analyzed by ICS. Shown are both absolute and relative percentages of positive CD8⁺ T cells within total CD8⁺ T cells from cultures treated with specific peptides subtracted values detected in CD8⁺ T cells from cultures treated with unrelated peptide. Values detected with splenocytes from mice injected with control vector were below the sensitivity threshold of the assay (not shown). The experimental data on which the analysis is based are the same as in FIGS. 7A, 7B, and 7C.

FIG. 10 shows intragroup mean percentages of IFN-γ, IL-2 and TNF-α positive CD8⁺ T cells within total CD8⁺ T cells from cultures of splenocytes isolated from each mouse injected (i.m.+EP) with the indicated DNA vectors as determined by detection of single, double, and triple cytokine positive CD8⁺ T cell sub-populations. Shown are both absolute and relative mean percentages of positive CD8⁺ T cells within total CD8⁺ T cells from cultures treated with specific peptides subtracted values detected in CD8⁺ T cells from cultures treated with unrelated peptide. Values detected with splenocytes from mice injected with control vector were below the sensitivity threshold of the assay (not shown). The experimental data on which the analysis is based are the same as in FIGS. 8A, 8B, and 8C.

FIGS. 11A and 11B show HPV16 E6-specific and E7-specific CD8⁺ and CD4⁺ T cell immunity induced in mice either injected or co-injected with the indicated DNA vectors, with FIG. 11A showing HPV16 E6-specific and E7-specific CD8⁺ T cell response and FIG. 11B showing HPV16 E6-specific and E7-specific CD4⁺ T cell response. C57BL/6 mice were inoculated (i.m.+EP) with 10 μg of the DNA vectors expressing Nef^{mut}/E6^{OD} and Nef^{mut}/E7^{OD} either separately or in combination. As control, mice were inoculated with the empty vector (Nil). At the time of sacrifice, 2.5×10⁵ splenocytes were incubated overnight with 5 μg/ml of unrelated (not shown), E6-specific, E7-specific, or E6+E7-specific peptides in triplicate IFN-γ ELISpot microwells. The number of IFN-γ spot-forming units (SFU)/well are reported as mean values of triplicates. Intragroup mean values are also reported. The E7-specific immune response alone was also evaluated in co-injected mice. ND: not done.

FIGS. 12A, 12B, and 12C show IFN-γ, IL-2 and TNF-α intracellular accumulation in CD8⁺ T cells from cultures of splenocytes isolated from each mouse injected (i.m.+EP) with the indicated DNA vectors, with FIG. 12A showing IFN-γ intracellular accumulation in CD8⁺ T cells, FIG. 12B showing IL-2 intracellular accumulation in CD8⁺ T cells, and FIG. 12C showing TNF-α intracellular accumulation in CD8⁺ T cells. Cryopreserved splenocytes were incubated overnight with 5 μg/ml of unrelated (not shown), E6-specific or E7-specific peptides in the presence of Brefeldin A, and then analyzed by ICS. Absolute percentages of positive CD8⁺ T cells within total CD8⁺ T cells for each cytokine subtracted values detected in CD8⁺ T cells from cultures treated with unrelated peptide are reported. N.D.: not detectable, values below the threshold sensitivity of the assay. In the histograms, each bar represents an individual animal.

FIGS. 13A, 13B, and 13C show intragroup mean values+SD of the percentages of IFN-γ, IL-2 and TNF-α intracellular accumulation in CD8⁺ T cells from cultures of splenocytes isolated from each mouse injected (i.m.+EP) with the indicated DNA vectors, with FIG. 13A showing intragroup mean values+SD of the percentages of IFN-γ intracellular accumulation in CD8⁺ T cells, FIG. 13B showing intragroup mean values+SD of the percentages of IL-2 intracellular accumulation in CD8⁺ T cells, and FIG. 13C showing intragroup mean values+SD of the percentages of TNF-α intracellular accumulation in CD8⁺ T cells. Shown are mean values+SD of the absolute percentages of positive CD8⁺ T cells within total CD8⁺ T cells from cultures treated with specific peptides subtracted values detected in CD8⁺ T cells from cultures treated with unrelated peptide. In the histograms, each bar represents a group of animals.

FIGS. 14A and 14B show IFN-γ, IL-2 and TNF-α intracellular accumulation in CD8⁺ T cells from cultures of splenocytes isolated from each mouse injected (i.m.+EP) with the indicated DNA vectors as determined by detection of single, double, and triple cytokine positive CD8⁺ T cell sub-populations, with FIG. 14A showing the results from mice with single injection of Nef^{mut}/E7^{OD} or Nef^{mut}/E6^{OD} and FIG. 14B showing the results from mice with co-injection of Nef^{mut}/E7^{OD}+Nef^{mut}/E6^{OD}. Cryopreserved splenocytes were incubated overnight with 5 μg/ml of unrelated (not shown), E6- or E7-specific peptides in the presence of Brefeldin A, and then analyzed by ICS. Shown are both absolute and relative percentages of positive CD8⁺ T cells within total CD8⁺ T cells from cultures treated with specific peptides subtracted values detected in CD8⁺ T cells from cultures treated with unrelated peptide. Values detected with splenocytes from mice injected with control vector were below the sensitivity threshold of the assay (not shown). The experimental data on which the analysis is based are the same as in FIGS. 12A, 12B, and 12C.

FIG. 15 shows intragroup mean percentages of IFN-γ, IL-2 and TNF-α positive CD8⁺ T cells within total CD8⁺ T cells from cultures of splenocytes isolated from each mouse injected (i.m.+EP) with the indicated DNA vectors as determined by detection of single, double, and triple cytokine positive CD8⁺ T cell sub-populations. Shown are both absolute and relative mean percentages of positive CD8⁺ T cells within total CD8⁺ T cells from cultures treated with specific peptides subtracted values detected in CD8⁺ T cells from cultures treated with unrelated peptide. Values detected with splenocytes from mice injected with control vector were below the sensitivity threshold of the assay (not shown). The experimental data on which the analysis is based are the same as in FIGS. 13A, 13B, and 13C.

FIGS. 16A and 16B show HPV16 E6-specific and E7-specific CD8⁺ and CD4⁺ T cell immunity induced in mice injected (i.m.+EP) with the indicated DNA vectors, with FIG. 16A showing HPV16 E6-specific and E7-specific CD8⁺ T cell response and FIG. 16B showing HPV16 E6-specific and E7-specific CD4⁺ T cell response. C57BL/6 mice were inoculated (i.m.+EP) with 10 μg of the DNA vectors expressing E6^{OD}, E7^{OD}, Nef^{mut}/E6^{OD}, or Nef^{mut}/E7^{OD}. As control, mice were inoculated with the empty vector (Nil). At the time of sacrifice, 2.5×10⁵ splenocytes were incubated overnight with 5 μg/ml of unrelated (not shown), E6, or E7-specific peptides in triplicate IFN-γ ELISpot microwells. Shown are the number of IFN-γ spot-forming units (SFU)/well as mean values of triplicates. Intragroup mean values are also reported.

FIGS. 17A, 17B, and 17C show IFN-γ, IL-2 and TNF-α intracellular accumulation in CD8⁺ T cells from cultures of splenocytes isolated from each mouse injected (i.m.+EP) with the indicated DNA vectors, with FIG. 17A showing IFN-γ intracellular accumulation in CD8⁺ T cells, FIG. 17B showing IL-2 intracellular accumulation in CD8⁺ T cells, and FIG. 17C showing TNF-α intracellular accumulation in CD8⁺ T cells. Cryopreserved splenocytes were incubated overnight with 5 μg/ml of unrelated (not shown), E6- or E7-specific peptides in the presence of Brefeldin A, and then analyzed by ICS. Absolute percentages of positive CD8⁺ T cells within total CD8⁺ T cells for each cytokine subtracted values detected in CD8⁺ T cells from cultures treated with unrelated peptide are reported. In the histograms, each bar represents an individual animal.

FIGS. 18A, 18B, and 18C show intragroup mean values+SD of the percentages of IFN-γ, IL-2 and TNF-α intracellular accumulation in CD8⁺ T cells from cultures of splenocytes isolated from each mouse injected (i.m.+EP) with the indicated DNA vectors, with FIG. 18A showing IFN-γ intracellular accumulation in CD8⁺ T cells, FIG. 18B showing IL-2 intracellular accumulation in CD8⁺ T cells, and FIG. 18C showing TNF-α intracellular accumulation in CD8⁺ T cells. Shown are mean values+SD of the absolute percentages of positive CD8⁺ T cells from cultures treated with specific peptides subtracted values detected in CD8⁺ T cells from cultures treated with unrelated peptide. In the histograms, each bar represents a group of animals.

FIGS. 19A and 19B show IFN-γ, IL-2 and TNF-α intracellular accumulation in CD8⁺ T cells within total CD8⁺ T cells from cultures of splenocytes isolated from each mouse injected (i.m.+EP) with the indicated DNA vectors as determined by detection of single, double, and triple cytokine positive CD8⁺ T cell sub-populations, with FIG. 19A showing the results from each mouse injected with Nef^{mut}/E7^{OD} or E7^{OD} and FIG. 19B showing the results from each mouse injected with Nef^{mut}/E6^{OD} or E6^{OD}. Cryopreserved splenocytes were incubated overnight with 5 μg/ml of unrelated (not shown), E6-specific or E7-specific peptides in the presence of Brefeldin A, and then analyzed by ICS. Shown are both absolute and relative percentages of positive CD8⁺ T cells within total CD8⁺ T cells from cultures treated with specific peptides subtracted values detected in CD8⁺ T cells from cultures treated with unrelated peptide. N.D.: values detected in CD8⁺ T cells from cultures treated with unrelated peptide were ≥ of those of CD8⁺ T cells from E6 peptide-treated cultures. Values detected with splenocytes from mice injected with control vector were below the sensitivity threshold of the assay (not shown). The experimental data on which the analysis is based are the same as in FIGS. 17A, 17B, and 17C.

FIG. 20 shows intragroup mean percentages of IFN-γ, IL-2 and TNF-α positive CD8⁺ T cells within total CD8⁺ T cells from cultures of splenocytes isolated from each mouse injected (i.m.+EP) with the indicated DNA vectors as determined by detection of single, double, and triple cytokine positive CD8⁺ T cell sub-populations. Shown are both absolute and relative mean percentages of positive CD8⁺ T cells within total CD8⁺ T cells from cultures treated with specific peptides subtracted values detected in CD8⁺ T cells from cultures treated with unrelated peptide. Values detected with splenocytes from mice injected with control vector were below the sensitivity threshold of the assay (not shown).

FIG. 21 shows the results of CTL assay carried out with CD8⁺ T cells isolated from mice inoculated (i.m.+EP) with the indicated vectors. The mean values of duplicates, calculated after subtraction of values obtained from co-cultures with CD8⁺ T cells isolated from mice injected with the empty vector, are shown. The experimental data on which the analysis is based are the same as in FIGS. 18A, 18B, and 18C.

FIG. 22 shows the HPV16 E6-specific and E7-specific CD8⁺ T cell immunity induced in tumor-bearing mice co-injected with the indicated DNA vectors. C57BL/6 mice were injected s.c. with 2×10⁵ TC-1 cells, and then inoculated (i.m.+EP) with 10 μg of both DNA vectors expressing E6^{OD} and E7^{OD} either alone or fused with Nef^{mut}. PBMCs were isolated after retro orbital bleeding, and then incubated overnight with 5 μg/ml of unrelated (not shown), or E6-specific and E7-specific nonamers in triplicate IFN-γ ELISpot microwells. Shown are the number of IFN-γ spot-forming units (SFU)/10⁵ PBMCs as mean values of triplicates. On the right, the SFU values are associated with each mouse. Intragroup mean values±SD are also reported.

FIGS. 23A, 23B, 23C, 23D, 23E, and 23F show the anti-tumor therapeutic effect induced by inoculation (i.m.+EP) of indicated DNA vectors, with FIG. 23A showing the cumulative data±SE as measured until the sacrifices of mice developing late tumors, FIG. 23B showing the data from each empty vector inoculated mouse, FIG. 23C showing the data from each Nef^{mut} inoculated mouse, FIG. 23D showing the data from each E6^{OD} and E7^{OD} inoculated mouse, FIG. 23E showing the data from each Nef^{mut}/E6^{OD} and Nef^{mut}/E7^{OD} inoculated mouse, and FIG. 23F showing the Kaplan-Meier survival curve. C57BL/6 mice (12 per group) were challenged with 2×10⁵ TC-1 cells and, the day after the tumor appearance, i.e., when tumor masses became detectable by palpation, co-inoculated with DNA vectors expressing Nef^{mut}/E6^{OD} and Nef^{mut}/E7^{OD}, E6^{OD} and E7^{OD}, or, as control, with either Nef^{mut} or empty vector. The DNA inoculations were repeated at day 17 after tumor cell implantation, and the growth of tumor mass was followed over time. Tumor sizes were measured every 2-3 days during the observation time. X-axis scale indicates the days of tumor monitoring, as well as the timing of tumor implantation, immunization and bleeding.

### NEW FIGURES

FIGS. 24A and 24B show the detection of Nef^{mut}/Ag85B and Nef^{mut}/ESAT-6 fusion in transfected cells (cell lysates) and respective exosomes, with FIG. 24A showing the fusion protein expression in transfected cells and FIG. 24B showing the fusion protein in exosomes.

FIGS. 25A and 25B illustrate the two different approaches used to produce Nef^{mut}/E7^{OD} mRNA for use as RNA vaccines, with FIG. 25A illustrating use of linearized plasmid as template for in vitro transcription and FIG. 25B showing use of a PCR amplicon as a template for in vitro transcription.

FIGS. 26A and 26B show the detection of Nef^{mut}/E7^{OD} fusion product in transfected cells and respective exosomes following mRNA delivery, with FIG. 26A showing the fusion protein expression in transfected cells and FIG. 26B showing the fusion protein in exosomes. As control, cells transfected with EGFP (Clontech, PT-3027-5 6085-1) were included. Mouse monoclonal anti-Nef antibodies served to detect Nef^{mut}/E7^{OD} product, while Vinculin and Alix were revealed as markers for cell lysates and exosomes, respectively. Relevant protein products are indicated by arrows. Molecular markers are provided in kDa.

FIGS. 27A and 27B show the detection of Nef^{mut}/E7^{OD} fusion product by mRNA delivery in transfected cells and respective exosomes with FIG. 27A showing the fusion protein expression in transfected cells and FIG. 27B showing the fusion protein in exosomes. As control, cells transfected with E7^{OD} mRNA were included. Mouse monoclonal anti-Nef antibodies served to detect Nef^{mut}/E7^{OD} product, mouse monoclonal anti E7 served to detect E7^{OD} product while Vinculin and Alix were revealed as markers for cell lysates and exosomes, respectively. Relevant protein products are indicated by arrows. Molecular markers are provided in kDa.

### 5. DETAILED DESCRIPTION

### 5.1. Definitions

Unless defined otherwise, all technical and scientific terms used herein have the meaning commonly understood by one of ordinary skill in the art to which the invention pertains.

As used herein, “Negative Regulatory Factor,” “Negative Factor,” or “Nef” refers to a small 27-35 kDa myristoylated protein encoded by primate lentiviruses, including Human Immunodeficiency Viruses (HIV-1 and HIV-2) and Simian Immunodeficiency Virus (SIV). HIV-1 Nef is a 27 kDa scaffold/adaptor protein that plays an important role in viral replication and pathogenesis. There are multiple HIV-1 Nef gene variants. Nef mutant (herein referred to as “Nef^{mut}”) is characterized by three amino acid substitutions in any of the known Nef gene variants: a glycine (G) to cysteine (C) substitution at position 3, a valine (V) to leucine (L) substitution at position 153, and a glutamate (E) to glycine (G) substitution at position 177. Nef^{mut} proteins are described in Lattanzi L. et al., 2012, Vaccine, 30: 7229-7237 and WO 2018/069947, each of which is incorporated herein by reference in its entirety. The amino acid sequence of an exemplary Nef^{mut} is disclosed below (SEQ ID NO: 1) with the amino acid substitutions as compared to wild type Nef underlined and in bold, and wherein “X” is V, L or I.

(SEQ ID NO: 1)
MGCKWSKSSVVGWPAVRERMRRAEPAADGVGAASRDLEKHGAITSSNTAA
TNADCAWLEAQEEEEVGFPVTPQVPLRPMTYKAAVDLSHFLKEKGGLEGL
IHSQRRQDILDLWIYHTQGYFPDWQNYTPGPGXRYPLTFGWCYKLVPVEP
EKLEEANKGENTSLLHPVSLHGMDDPGREVLEWRFDSRLAFHHVARELHP
EYFKNC

As used herein, the term “extracellular vesicle (EV)” refers to a cell-derived vesicle comprising a membrane that encloses an internal space. Extracellular vesicles comprise all membrane-bound vesicles that have a smaller diameter than the cell from which they are derived. Generally, extracellular vesicles can comprise various macromolecular cargo either within the internal space, displayed on the external surface of the extracellular vesicle, and/or spanning the membrane. The cargo can comprise nucleic acids, proteins, carbohydrates, lipids, small molecules, and/or combinations thereof. By way of example and without limitation, extracellular vesicles include apoptotic bodies, microvesicles, exosomes, and nanovesicles. Extracellular vesicles can be derived from a living or dead organism, explanted tissues or organs, and/or cultured cells. Extracellular vesicles can be engineered in vivo or in vitro to incorporate antigens.

As used herein the term “exosome” refers to an extracellular vesicle that is between 30-150 nm in diameter, typically 50-100 nm in diameter, comprising a membrane that encloses an internal space, and which is generated from the cell by inward budding of late endosomes and release from the cell by fusion of multivesicular bodies (MVBs) with the plasma membrane. For use in a vaccine product, the exosome can be derived from a producer cell, and isolated from the producer cell based on its size, density, biochemical parameters, or a combination thereof. Exosomes can be engineered in vivo or in vitro to incorporate antigens.

As used herein, the term “nanovesicle” refers to an extracellular vesicle between 20-250 nm in diameter, typically 30-150 nm in diameter, comprising a membrane that encloses an internal space, and which is generated from the cell by direct or indirect manipulation such that the nanovesicle would not be produced by the producer cell without the manipulation. Appropriate manipulations of the producer cell include but are not limited to serial extrusion, treatment with alkaline solutions, sonication, or combinations thereof. The production of nanovesicles can, in some instances, result in the destruction of the producer cell. The nanovesicle, once it is derived from a producer cell according to the manipulation, can be isolated from the producer cell based on its size, density, biochemical parameters, or a combination thereof. Nanovesicles can be engineered in vivo or in vitro to incorporate antigens.

As used herein, the term “nanoparticle” refers to an engineered particle ranging from 1 to 500 nm in diameter, typically 1 to 100 nm in diameter. Nanoparticles can be generated from biological and/or chemical materials, such as phospholipids, lipids, lactic acid, dextran, chitosan, polymers, carbon, silica, and metal. The medical applications of engineered nanoparticles include drug delivery and in vivo or in vitro diagnostics.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared. If not otherwise specified, percentage identities herein are determined using BLAST with NCBI default parameters.

By “subject” is meant a human or non-human mammal including, but not limited to, bovine, equine, canine, ovine, feline, and rodent, including murine and rattus, subjects. A “patient” is a human subject.

5.2. Fusion Protein

In a first aspect, provided herein is a fusion protein. The fusion protein comprises from N-terminus to C-terminus, (a) an exosome-anchoring polypeptide domain and (b) an immunogenic antigen polypeptide domain.

In some embodiments, the N-terminus of the immunogenic antigen polypeptide domain is directly fused to the C-terminus of the exosome-anchoring polypeptide domain. In some other embodiments, the N-terminus of the immunogenic antigen polypeptide domain is fused to the C-terminus of the exosome-anchoring polypeptide domain via a peptide linker.

5.2.1. Exosome-Anchoring Polypeptide Domain

The fusion protein disclosed herein comprises an exosome-anchoring polypeptide domain.

In some embodiments, the exosome-anchoring polypeptide is a Nef^{mut} protein (SEQ ID NO: 1). In some embodiments, the exosome-anchoring polypeptide has 90%, 91%, 92%, 93%, 94%, 95%, 96%, 97%, 98%, or 99% sequence identity to the full length of amino acid sequence of SEQ ID NO: 1, wherein the amino acid residues at position 3, position 153, and position 177 of SEQ ID NO: 1 are maintained.

In some embodiments, the exosome-anchoring polypeptide is a truncated Nef^{mut} protein. In particular embodiments, the truncated Nef^{mut} protein retains the amino acid residues at position 3, position 153, and position 177 of SEQ ID NO:1.

In certain embodiments, the exosome-anchoring polypeptide has an amino acid sequence selected from SEQ ID NOs: 2-30. In some embodiments, the exosome-anchoring polypeptide has 90%, 91%, 92%, 93%, 94%, 95%, 96%, 97%, 98%, or 99% sequence identity to the full length of an amino acid sequence selected from SEQ ID NOs: 2-30, wherein the amino acid residues at position 3, position 153, and position 177 of SEQ ID NOs: 2-30 are maintained.

In certain embodiments, the exosome-anchoring polypeptide has the amino acid sequence of SEQ ID NO: 30. The truncated Nef^{mut} protein having the amino acid sequence of SEQ ID NO: 30 is herein referred to as Nef^{mut}PL. In certain embodiments, the exosome-anchoring polypeptide has 90%, 91%, 92%, 93%, 94%, 95%, 96%, 97%, 98%, or 99% sequence identity to the full length of the amino acid sequence of SEQ ID NO: 30, wherein the amino acid residues at position 3, position 153, and position 177 of SEQ ID NO: 30 are maintained. The amino acid sequences of various truncated Nef^{mut} proteins are shown in Table 1 below, wherein “X” is V, L or I.

TABLE 1

| SEQ ID NO: | Amino Acid Sequence |
|---|---|
| 2 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHPEYFKN |
| 3 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHPEYFK |
| 4 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHPEYF |
| 5 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHPEY |
| 6 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHPE |
| 7 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELHP |
| 8 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARELH |
| 9 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVAREL |
| 10 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVARE |
| 11 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVAR |
| 12 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHVA |
| 13 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHHV |
| 14 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFHH |
| 15 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAFH |
| 16 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLAF |
| 17 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRLA |
| 18 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSRL |
| 19 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDSR |
| 20 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFDS |
| 21 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRFD |
| 22 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWRF |
| 23 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEWR |
| 24 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LEW |
| 25 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | LE |
| 26 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| | L |
| 27 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGREV |
| 28 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGRE |
| 29 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPGR |
| 30 | MGCKWSKSSVVGWPAVRERMRRAEPAADGVGAASRDLEKHGAITSSNTAATNADCAWLEA |
| | QEEEEVGFPVTPQVPLRPMTYKAAVDLSHFLKEKGGLEGLIHSQRRQDILDLWIYHTQGY |
| | FPDWQNYTPGPGXRYPLTFGWCYKLVPVEPEKLEEANKGENTSLLHPVSLHGMDDPG |

In certain embodiments, the full length Nef^{mut} protein is encoded by the polynucleotide having the nucleic acid sequence of SEQ ID NO: 31, wherein “XXX” is a tri nucleotide sequence that forms a codon that corresponds to valine, leucine or isoleucine. In certain embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 31)
ATGGGTTGCAAGTGGTCAAAAAGTAGTGTGGTTGGATGGCCTGCTGTAAG
GGAAAGAATGAGACGAGCTGAGCCAGCAGCAGATGGGGTGGGAGCAGCAT
CTCGAGACCTAGAAAAACATGGAGCAATCACAAGTAGCAATACAGCAGCT
ACCAATGCTGATTGTGCCTGGCTAGAAGCACAAGAGGAGGAGGAGGTGGG
TTTTCCAGTCACACCTCAGGTACCTTTAAGACCAATGACTTACAAGGCAG
CTGTAGATCTTAGCCACTTTTTAAAAGAAAAGGGGGGACTGGAAGGGCTA
ATTCACTCCCAACGAAGACAAGATATCCTTGATCTGTGGATCTACCACAC
ACAAGGCTACTTCCCTGATTGGCAGAACTACACACCAGGACCAGGGXXXA
GATATCCACTGACCTTTGGATGGTGCTACAAGCTAGTACCAGTTGAGCCA
GAGAAGTTAGAAGAAGCCAACAAAGGAGAGAACACCAGCTTGTTACACCC
TGTGAGCCTGCATGGAATGGATGACCCGGGGAGAGAAGTGTTAGAGTGGA
GGTTTGACAGCCGCCTAGCATTTCATCACGTGGCCCGAGAGCTGCATCCG
GAGTACTTCAAGAACTGCTGA

In certain embodiments, the Nef^{mut}PL truncated protein is encoded by the polynucleotide having the nucleic acid sequence of SEQ ID NO: 32, wherein “XXX” is a tri nucleotide sequence that forms a codon that corresponds to valine, leucine or isoleucine. In certain embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 32)
ATGGGTTGCAAGTGGTCAAAAAGTAGTGTGGTTGGATGGCCTGCTGTAAG
GGAAAGAATGAGACGAGCTGAGCCAGCAGCAGATGGGGTGGGAGCAGCAT
CTCGAGACCTAGAAAAACATGGAGCAATCACAAGTAGCAATACAGCAGCT
ACCAATGCTGATTGTGCCTGGCTAGAAGCACAAGAGGAGGAGGAGGTGGG
TTTTCCAGTCACACCTCAGGTACCTTTAAGACCAATGACTTACAAGGCAG
CTGTAGATCTTAGCCACTTTTTAAAAGAAAAGGGGGGACTGGAAGGGCTA
ATTCACTCCCAACGAAGACAAGATATCCTTGATCTGTGGATCTACCACAC
ACAAGGCTACTTCCCTGATTGGCAGAACTACACACCAGGACCAGGGXXXA
GATATCCACTGACCTTTGGATGGTGCTACAAGCTAGTACCAGTTGAGCCA
GAGAAGTTAGAAGAAGCCAACAAAGGAGAGAACACCAGCTTGTTACACCC
TGTGAGCCTGCATGGAATGGATGACCCGGGG

5.2.2. Immunogenic Antigen Polypeptide Domain

The fusion protein disclosed herein comprises an immunogenic antigen polypeptide domain.

In some embodiments, the immunogenic antigen is a virus antigen, a bacteria antigen, a fungal antigen, a eukaryotic parasite antigen, or a tumor antigen.

In some embodiments, the immunogenic antigen is a full-length protein from a virus, bacterium, fungus, parasite, or tumor. In some embodiments, the immunogenic antigen is a fragment of a protein from a virus, bacterium, fungus, parasite, or tumor. In certain embodiments, the immunogenic antigen is an N-terminal fragment of the protein from a virus, bacterium, fungus, parasite, or tumor. In certain embodiments, the immunogenic antigen is a C-terminal fragment protein from a virus, bacterium, fungus, parasite, or tumor.

In some embodiments, the immunogenic antigen is “detoxified”—i.e., the sequence is altered to reduce or abrogate binding to host cell proteins.

5.2.2.1. Virus Antigen

In some embodiments, the immunogenic antigen is a virus antigen. In some embodiments, the virus antigen is a viral structural protein, such as a capsid protein, an envelope protein, or a membrane protein. In some embodiments, the virus antigen is a viral nonstructural protein, such as a viral enzyme.

In various embodiments, the virus antigen is selected from the group consisting of: a human papillomavirus (HPV) antigen, a human immunodeficiency virus (HIV) antigen, a hepatitis B virus (HBV) antigen, a hepatitis C virus (HCV) antigen, an Ebola virus antigen, a West Nile virus antigen, a Crimean-Congo virus antigen, a dengue virus antigen, and an influenza virus antigen.

In some embodiments, the virus antigen is a human papillomavirus (HPV) antigen. In certain embodiments, the HPV antigen is E6 or E7 of human papillomavirus.

In some embodiments, the virus antigen is a human immunodeficiency virus (HIV) antigen. In certain embodiments, the HIV antigen is Gag or Tat of human immunodeficiency virus.

In some embodiments, the virus antigen is a hepatitis B virus (HBV) antigen. In certain embodiments, the HBV antigen is Core of hepatitis B virus.

In some embodiments, the virus antigen is a hepatitis C virus (HCV) antigen. In certain embodiments, the HCV antigen is Core, NS3, E1, or E2 of hepatitis C virus.

In some embodiments, the virus antigen is an Ebola virus antigen. In certain embodiments, the Ebola virus antigen is VP24, VP40, NP, or GP of Ebola virus.

In some embodiments, the virus antigen is a West Nile virus antigen. In certain embodiments, the West Nile virus antigen is NS3 of West Nile virus.

In some embodiments, the virus antigen is a Crimean-Congo virus antigen. In certain embodiments, the Crimean-Congo virus antigen is GP or NP of Crimean-Congo virus.

In some embodiments, the virus antigen is a dengue virus antigen.

In some embodiments, the virus antigen is an influenza virus antigen. In various embodiments, the influenza virus is selected from the group consisting of parainfluenza virus 1, parainfluenza virus 2, influenza A virus, and influenza B virus. In certain embodiments, the influenza virus is influenza A virus. In particular embodiments, the virus antigen is the nucleoprotein (NP) or the matrix protein (M1) of influenza A virus.

5.2.2.1.1. HPV Antigen

In some embodiments, the virus antigen is a human papillomavirus (HPV) antigen. In various embodiments, the HPV is selected from HPV16, HPV18, HPV6, or HPV11. In some embodiments, the HPV is HPV16 or HPV18. In certain embodiments, the HPV is HPV16. In certain embodiments, the HPV is HPV18. In some embodiments, the HPV antigen is a structural protein, such as a capsid protein. In some other embodiments, the HPV antigen is a nonstructural protein. In some embodiments, the HPV antigen is selected from the group consisting of L1, L2, E1, E2, E3, E4, E5, E6, and E7 of human papillomavirus. In certain embodiments, the HPV antigen is E6 or E7 of human papillomavirus. In some of these embodiments, the HPV antigen is detoxified to reduce or abrogate binding to host cell proteins. In certain embodiments, the HPV antigen is detoxified to remove a p53 binding site. In certain embodiments, the HPV antigen is detoxified to remove a retinoblastoma protein (pRB) binding site.

In some embodiments, the HPV antigen is selected from the group consisting of L1, L2, E1, E2, E3, E4, E5, E6, and E7 of HPV16. In certain embodiments, the HPV antigen is E6 or E7 of HPV16.

In some embodiments, the immunogenic antigen is E6 of HPV16. In certain embodiments, the open reading frame (ORF) of E6 is detoxified. In particular embodiments, the amino acid sequence of detoxified E6 of HPV16 is SEQ ID NO: 33.

(SEQ ID NO: 33)
MHQKRTAMFQDPQERPRKLPQLCTELQTTIHDIILECVYCKQQLLRREVY
DFAFRDLCIVYRDGSPYAVCDKCLKFYSKISEYRHYCYSLYGTTLEQQYN
KPLCDLLIRCINCQKPLCPEEKQRHLDKKQRFHNIRVRWTGRCMSCCRSS
RTRRETQL

In certain embodiments, the nucleic acid sequence encoding detoxified E6 of HPV16 (E6^{DETOX}) is SEQ ID NO: 34.

(SEQ ID NO: 34)
ATGCACCAAAAGAGAACTGCAATGTTTCAGGACCCACAGGAGCGACCCAG
AAAGTTACCACAGTTATGCACAGAGCTGCAAACAACTATACATGATATAA
TATTAGAATGTGTGTACTGCAAGCAACAGTTACTGCGACGTGAGGTATAT
GACTTTGCTTTTCGGGATTTATGCATAGTATATAGAGATGGGAATCCATA
TGCTGTATGTGATAAATGTTTAAAGTTTTATTCTAAAATTAGTGAGTATA
GACATTATTGTTATAGTTTGTATGGAACAACATTAGAACAGCAATACAAC
TAAACCGTTGTGTGATTGTTAATTAGGTGTATTAACTGTCAAAAGCCACT
GGTGTCCTGAAGAAAAGCAAAGACATCTGACAAAAAGCAAAGATTCCATA
ATATAAGGGTGCGGTGGACCGGTCGATGTATGTCTTGTTGCAGATCATCA
AGAACACGTAGAGAAACCCAGCTGTAA

In certain embodiments, the nucleic acid sequence encoding detoxified E6 of HPV16 is codon optimized for expression in patients. In specific embodiments, the nucleic acid sequence encoding codon optimized and detoxified E6 of HPV16 (E6^{OD}) is SEQ ID NO: 35.

(SEQ ID NO: 35)
ATGCACCAGAAGCGCACCGCCATGTTCCAGGACCCCCAGGAGCGCCCCCG
CAAGCTGCCCCAGCTGTGCACCGAGCTGCAGACCACCATCCACGACATCA
TCCTGGAGTGCGTGTACTGCAAGCAGCAGCTGCTGCGCCGCGAGGTGTAC
GACTTCGCCTTCCGCGACCTGTGCATCGTGTACCGCGACGGCAGCCCCTA
CGCCGTGTGCGACAAGTGCCTGAAGTTCTACTCCAAGATCAGCGAGTACC
GCCACTACTGCTACAGCCTGTACGGCACCACCCTGGAGCAGCAGTACAAC
AAGCCCCTGTGCGACCTGCTGATCCGCTGCATCAACTGCCAGAAGCCCCT
GTGCCCCGAGGAGAAGCAGCGCCACCTGGACAAGAAGCAGCGCTTCCACA
ACATCCGGGTGCGGTGGACCGGGCGCTGCATGAGCTGCTGCCGCAGCAGC
CGCACCCGCCGCGAGACCCAGCTGTAA

In some embodiments, the immunogenic antigen is E7 of HPV16. In certain embodiments, the open reading frame (ORF) of E7 is detoxified. In particular embodiments, the amino acid sequence of detoxified E7 of HPV16 is SEQ ID NO: 36.

(SEQ ID NO: 36)
MHGDTPTLHEYMLDLQPETTGLYGYGQLNDSSEEEDEIDGPAGQAEPDRA
HYNIVTFCCKCDSTLRLCVQSTHVDIRTLEDLLMGTLGIVCPICSQKP

In certain embodiments, the nucleic acid sequence encoding detoxified E7 of HPV16 (E7^{DETOX}) is SEQ ID NO: 37.

(SEQ ID NO: 37)
ATGCATGGAGATACACCTACATTGCATGAATATATGTTAGATTTGCAACC
AGAGACAACTGGCCTCTACGGCTATGGCCAATTAAATGACAGCTCAGAGG
AGGAGGATGAAATAGATGGTCCAGCTGGACAAGCAGAACCGGACAGAGCC
CATTACAATATTGTAACCTTTTGTTGCAAGTGTGACTCTACGCTTCGGTT
GTGCGTACAAAGCACACACGTAGACATTCGTACTTTGGAAGACCTGTTAA
TGGGCACACTAGGAATTGTGTGCCCCATCTGTTCTCAGAAACCATAA

In certain embodiments, the nucleic acid sequence encoding detoxified E7 of HPV16 is codon optimized. In specific embodiments, the nucleic acid sequence encoding codon optimized and detoxified E7 of HPV16 (E7^{OD}) is SEQ ID NO: 38.

(SEQ ID NO: 38)
ATGCACGGCGACACCCCCACCTTGCACGAGTACATGTTGGACTTGCAGCCCGAGACCACCGGC
CTGTACGGCTACGGCCAGTTGAACGACAGCTCCGAGGAGGAGGACGAGATCGACGGCCCCGCC
GGCCAGGCCGAGCCCGACCGCGCCCACTACAACATCGTGACCTTCTGCTGCAAGTGCGACTCC
ACCCTGCGCCTGTGCGTGCAGAGCACCCACGTGGACATCCGCACCTTGGAGGACCTGCTGATG
GGCACCCTGGGCATCGTGTGCCCCATCTGCAGCCAGAAGCCCTAA

In certain embodiments, the fusion protein comprises from N-terminus to C-terminus a Nef^{mut} protein and a detoxified E6 of HPV16. In some embodiments, the C-terminus of the Nef^{mut} protein and the N-terminus of the detoxified E6 of HPV16 are fused directly without a linker. In particular embodiments, the fusion protein has the amino acid sequence of SEQ ID NO: 39, wherein “X” is V, L or I.

(SEQ ID NO: 39)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGREVLEWRFDSRL
AFHHVARELHPEYFKNCGPGPHQKRTAMFQDPQERPRKLPQLCTELQTTIHDIILECVYCKQQ
LLRREVYDFAFRDLCIVYRDGSPYAVCDKCLKFYSKISEYRHYCYSLYGTTLEQQYNKPLCDL
LIRCINCQKPLCPEEKQRHLDKKQRFHNIRVRWTGRCMSCCRSSRTRRETQL

In certain embodiments, the nucleic acid encoding the fusion protein (Nef^{mut}/E6^{DETOX}) is SEQ ID NO: 40, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 40)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
CACCAAAAGAGAACTGCAATGTTTCAGGACCCACAGGAGCGACCCAGAAAGTTACCACAGTTA
TGCACAGAGCTGCAAACAACTATACATGATATAATATTAGAATGTGTGTACTGCAAGCAACAG
TTACTGCGACGTGAGGTATATGACTTTGCTTTTCGGGATTTATGCATAGTATATAGAGATGGG
AATCCATATGCTGTATGTGATAAATGTTTAAAGTTTTATTCTAAAATTAGTGAGTATAGACAT
TATTGTTATAGTTTGTATGGAACAACATTAGAACAGCAATACAACAAACCGTTGTGTGATTTG
TTAATTAGGTGTATTAACTGTCAAAAGCCACTGTGTCCTGAAGAAAAGCAAAGACATCTGGAC
AAAAAGCAAAGATTCCATAATATAAGGGTGCGGTGGACCGGTCGATGTATGTCTTGTTGCAGA
TCATCAAGAACACGTAGAGAAACCCAGCTGTAA

In certain embodiments, the nucleic acid sequence encoding detoxified E6 of HPV16 of the fusion protein is codon optimized. In specific embodiments, the nucleic acid sequence encoding the fusion protein (Nef^{mut}/E6^{OD}) is SEQ ID NO: 41, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 41)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
CACCAGAAGCGCACCGCCATGTTCCAGGACCCCCAGGAGCGCCCCCGCAAGCTGCCCCAGCTG
TGCACCGAGCTGCAGACCACCATCCACGACATCATCCTGGAGTGCGTGTACTGCAAGCAGCAG
CTGCTGCGCCGCGAGGTGTACGACTTCGCCTTCCGCGACCTGTGCATCGTGTACCGCGACGGC
AGCCCCTACGCCGTGTGCGACAAGTGCCTGAAGTTCTACTCCAAGATCAGCGAGTACCGCCAC
TACTGCTACAGCCTGTACGGCACCACCCTGGAGCAGCAGTACAACAAGCCCCTGTGCGACCTG
CTGATCCGCTGCATCAACTGCCAGAAGCCCCTGTGCCCCGAGGAGAAGCAGCGCCACCTGGAC
AAGAAGCAGCGCTTCCACAACATCCGGGTGCGGTGGACCGGGCGCTGCATGAGCTGCTGCCGC
AGCAGCCGCACCCGCCGCGAGACCCAGCTGTAA

In certain embodiments, the fusion protein comprises from N-terminus to C-terminus a Nef^{mut} protein and a detoxified E7 of HPV16. In some embodiments, the C-terminus of the Nef^{mut} protein and the N-terminus of the detoxified E7 of HPV16 are fused directly without a linker. In particular embodiments, the fusion protein has the amino acid sequence of SEQ ID NO: 42, wherein “X” is V, L or I

(SEQ ID NO: 42)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGREVLEWRFDSRL
AFHHVARELHPEYFKNCGPGPHGDTPTLHEYMLDLQPETTGLYGYGQLNDSSEEEDEIDGPAG
QAEPDRAHYNIVTFCCKCDSTLRLCVQSTHVDIRTLEDLLMGTLGIVCPICSQKP

In certain embodiments, the nucleic acid encoding the fusion protein (Nef^{mut}/E7^{DETOX}) is SEQ ID NO: 43, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 43)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
CATGGAGATACACCTACATTGCATGAATATATGTTAGATTTGCAACCAGAGACAACTGGCCTC
TACGGCTATGGCCAATTAAATGACAGCTCAGAGGAGGAGGATGAAATAGATGGTCCAGCTGGA
CAAGCAGAACCGGACAGAGCCCATTACAATATTGTAACCTTTTGTTGCAAGTGTGACTCTACG
CTTCGGTTGTGCGTACAAAGCACACACGTAGACATTCGTACTTTGGAAGACCTGTTAATGGGC
ACACTAGGAATTGTGTGCCCCATCTGTTCTCAGAAACCATAA

In certain embodiments, the nucleic acid sequence encoding detoxified E7 of HPV16 of the fusion protein is codon optimized. In specific embodiments, the nucleic acid sequence encoding the fusion protein (Nef^{mut}/E7^{OD}) is SEQ ID NO: 44, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 44)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
CACGGCGACACCCCCACCTTGCACGAGTACATGTTGGACTTGCAGCCCGAGACCACCGGCCTG
TACGGCTACGGCCAGTTGAACGACAGCTCCGAGGAGGAGGACGAGATCGACGGCCCCGCCGGC
CAGGCCGAGCCCGACCGCGCCCACTACAACATCGTGACCTTCTGCTGCAAGTGCGACTCCACC
CTGCGCCTGTGCGTGCAGAGCACCCACGTGGACATCCGCACCTTGGAGGACCTGCTGATGGGC
ACCCTGGGCATCGTGTGCCCCATCTGCAGCCAGAAGCCCTAA

In certain embodiments, the fusion protein comprises from N-terminus to C-terminus a truncated Nef^{mut} protein and a detoxified E6 of HPV16. In some embodiments, the C-terminus of the truncated Nef^{mut} protein and the N-terminus of the detoxified E6 of HPV16 are fused directly without a linker. In certain embodiments, the truncated Nef^{mut} protein is Nef^{mut}PL. In particular embodiments, the fusion protein has the amino acid sequence of SEQ ID NO: 45, wherein “X” is V, L or I.

(SEQ ID NO: 45)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGGPGPHQKRTAMF
QDPQERPRKLPQLCTELQTTIHDIILECVYCKQQLLRREVYDFAFRDLCIVYRDGSPYAVCDK
CLKFYSKISEYRHYCYSLYGTTLEQQYNKPLCDLLIRCINCQKPLCPEEKQRHLDKKQRFHNI
RVRWTGRCMSCCRSSRTRRETQL

In certain embodiments, the nucleic acid encoding the fusion protein (Nef^{mut}PL/E6^{DETOX}) is SEQ ID NO: 46, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 46)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGGGACCTGGGCCCCACCAAAAGAGAACTGCAATGTTT
CAGGACCCACAGGAGCGACCCAGAAAGTTACCACAGTTATGCACAGAGCTGCAAACAACTATA
CATGATATAATATTAGAATGTGTGTACTGCAAGCAACAGTTACTGCGACGTGAGGTATATGAC
TTTGCTTTTCGGGATTTATGCATAGTATATAGAGATGGGAATCCATATGCTGTATGTGATAAA
TGTTTAAAGTTTTATTCTAAAATTAGTGAGTATAGACATTATTGTTATAGTTTGTATGGAACA
ACATTAGAACAGCAATACAACAAACCGTTGTGTGATTTGTTAATTAGGTGTATTAACTGTCAA
AAGCCACTGTGTCCTGAAGAAAAGCAAAGACATCTGGACAAAAAGCAAAGATTCCATAATATA
AGGGTGCGGTGGACCGGTCGATGTATGTCTTGTTGCAGATCATCAAGAACACGTAGAGAAACC
CAGCTGTAA

In certain embodiments, the nucleic acid sequence encoding detoxified E6 of HPV16 of the fusion protein is codon optimized. In specific embodiments, the nucleic acid sequence encoding the fusion protein (Nef^{mut}PL/E6^{OD}) is SEQ ID NO: 47, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 47)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGOTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGGGACCTGGGCCCCACCAGAAGCGCACCGCCATGTTC
CAGGACCCCCAGGAGCGCCCCCGCAAGCTGCCCCAGCTGTGCACCGAGCTGCAGACCACCATC
CACGACATCATCCTGGAGTGCGTGTACTGCAAGCAGCAGCTGCTGCGCCGCGAGGTGTACGAC
TTCGCCTTCCGCGACCTGTGCATCGTGTACCGCGACGGCAGCCCCTACGCCGTGTGCGACAAG
TGCCTGAAGTTCTACTCCAAGATCAGCGAGTACCGCCACTACTGCTACAGCCTGTACGGCACC
ACCCTGGAGCAGCAGTACAACAAGCCCCTGTGCGACCTGCTGATCCGCTGCATCAACTGCCAG
AAGCCCCTGTGCCCCGAGGAGAAGCAGCGCCACCTGGACAAGAAGCAGCGCTTCCACAACATC
CGGGTGCGGTGGACCGGGCGCTGCATGAGCTGCTGCCGCAGCAGCCGCACCCGCCGCGAGACC
CAGCTGTAA

In certain embodiments, the fusion protein comprises from N-terminus to C-terminus a truncated Nef^{mut} protein and a detoxified E7 of HPV16. In some embodiments, the C-terminus of the truncated Nef^{mut} protein and the N-terminus of the detoxified E7 of HPV16 are fused directly without a linker. In certain embodiments, the truncated Nef^{mut} protein is Nef^{mut}PL. In particular embodiments, the fusion protein has the amino acid sequence of SEQ ID NO: 48, wherein “X” is V, L or I.

(SEQ ID NO: 48)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGGPGPHGDTPTLH
EYMLDLQPETTGLYGYGQLNDSSEEEDEIDGPAGQAEPDRAHYNIVTFCCKCDSTLRLCVQST
HVDIRTLEDLLMGTLGIVCPICSQKP

In certain embodiments, the nucleic acid encoding the fusion protein (Nef^{mut}PL/E7^{DETOX}) is SEQ ID NO: 49, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 49)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGGGACCTGGGCCCCATGGAGATACACCTACATTGCAT
GAATATATGTTAGATTTGCAACCAGAGACAACTGGCCTCTACGGCTATGGCCAATTAAATGAC
AGCTCAGAGGAGGAGGATGAAATAGATGGTCCAGCTGGACAAGCAGAACCGGACAGAGCCCAT
TACAATATTGTAACCTTTTGTTGCAAGTGTGACTCTACGCTTCGGTTGTGCGTACAAAGCACA
CACGTAGACATTCGTACTTTGGAAGACCTGTTAATGGGCACACTAGGAATTGTGTGCCCCATC
TGTTCTCAGAAACCATAA

In certain embodiments, the nucleic acid sequence encoding detoxified E7 of HPV16 of the fusion protein is codon optimized. In specific embodiments, the nucleic acid sequence encoding the fusion protein (Nef^{mut}PL/E7^{OD}) is SEQ ID NO: 50, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 50)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGCTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGGGACCTGGGCCCCACGGCGACACCCCCACCTTGCAC
GAGTACATGTTGGACTTGCAGCCCGAGACCACCGGCCTGTACGGCTACGGCCAGTTGAACGAC
AGCTCCGAGGAGGAGGACGAGATCGACGGCCCCGCCGGCCAGGCCGAGCCCGACCGCGCCCAC
TACAACATCGTGACCTTCTGCTGCAAGTGCGACTCCACCCTGCGCCTGTGCGTGCAGAGCACC
CACGTGGACATCCGCACCTTGGAGGACCTGCTGATGGGCACCCTGGGCATCGTGTGCCCCATC
TGCAGCCAGAAGCCCTAA

5.2.2.2. Bacteria Antigen

In some embodiments, the immunogenic antigen is a bacteria antigen.

In some embodiments, the bacteria antigen is a Mycobacterium tuberculosis antigen. In some embodiments, the Mycobacterium tuberculosis antigen is Ag85B or ESAT-6. In some embodiments, the Mycobacterium tuberculosis antigen comprises SEQ ID NO: 51 or SEQ ID NO: 75.

5.2.2.2.1. TB Antigen

In some embodiments, the bacterial antigen is a human tuberculosis (TB) antigen. In some embodiments, the TB antigen is Rv1196; Rv0125; ESAT-6; Ag85B; TB10.4; Ag85B; H1+Rv2660c; Rv2608; Rv3619; Rv3620; Rv1813; Antigen 85A; Antigen 85A; Antigen 85A; TB10.4; Antigen 85B; or Antigen 85A. In certain embodiments, the TB antigen is antigen 85B (Ag85B) of Mycobacterium tuberculosis. In certain embodiments, the TB antigen is antigen 85A (Ag85A) of Mycobacterium tuberculosis. In certain embodiments, the TB antigen is Early secretory antigenic target-6 (ESAT-6) of Mycobacterium tuberculosis.

In some embodiments, the immunogenic antigen is Ag85B. In particular embodiments, the amino acid sequence of Ag85B is SEQ ID NO: 51.

(SEQ ID NO: 51)
MTDVSRKIRAWGRRLMIGTAAAVVLPGLVGLAGGAATAGAFSRPGLPVEYLQVPSPSMGRDIK
VQFQSGGNNSPAVYLLDGLRAQDDYNGWDINTPAFEWYYQSGLSIVMPVGGQSSFYSDWYSPA
CGKAGCQTYKWETFLTSELPQWLSANRAVKPTGSAAIGLSMAGSSAMILAAYHPQQFIYAGSL
SALLDPSQGMGPSLIGLAMGDAGGYKAADMWGPSSDPAWERNDPTQQIPKLVANNTRLWVYCG
NGTPNELGGANIPAEFLENFVRSSNLKFQDAYNAAGGHNAVFNFPPNGTHSWEYWGAQLNAMK
GDLQSSLGAG

In some embodiments, the immunogenic antigen is Early secretory antigenic target-6 (ESAT-6) of Mycobacterium tuberculosis. In particular embodiments, the amino acid sequence of ESAT-6 is SEQ ID NO: 75.

(SEQ ID NO: 75)
MTEQQWNFAGIEAAASAIQGNVTSIHSLLDEGKQSLTKLAAAWGGSGS
EAYQGVQQKWDATATELNNALQNLARTISEAGQAMASTEGNVTGMFA

In certain embodiments, the fusion protein comprises from N-terminus to C-terminus a Nef^{mut} protein and Ag85B. In some embodiments, the C-terminus of the Nef^{mut} protein and the N-terminus of the Ag85B are fused without the Ag85B signal sequence. In particular embodiments, the fusion protein has the amino acid sequence of SEQ ID NO: 85, wherein “X” is V, L or I.

(SEQ ID NO: 85)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGREVLEWRFDSRL
AFHHVARELHPEYFKNCGPGPFSRPGLPVEYLQVPSPSMGRDIKVQFQSGGNNSPAVYLLDGL
RAQDDYNGWDINTPAFEWYYQSGLSIVMPVGGQSSFYSDWYSPACGKAGCQTYKWETFLTSEL
PQWLSANRAVKPTGSAAIGLSMAGSSAMILAAYHPQQFIYAGSLSALLDPSQGMGPSLIGLAM
GDAGGYKAADMWGPSSDPAWERNDPTQQIPKLVANNTRLWVYCGNGTPNELGGANIPAEFLEN
FVRSSNLKFQDAYNAAGGHNAVFNFPPNGTHSWEYWGAQLNAMKGDLQSSLGAG

In certain embodiments, the nucleic acid encoding the fusion protein (Nef^{mut}/Ag85B) is SEQ ID NO: 86, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 86)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGOTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
TTTAGCAGGCCTGGCCTGCCCGTGGAATACCTGCAAGTGCCTTCCCCTAGCATGGGAAGAGAT
ATTAAGGTGCAGTTCCAGAGCGGCGGAAACAACAGCCCCGCTGTTTACCTGCTCGACGGCCTG
AGAGCCCAGGATGACTACAACGGCTGGGACATCAACACCCCTGCCTTCGAGTGGTACTACCAG
TCTGGCCTGTCTATCGTGATGCCAGTGGGCGGCCAGAGCAGCTTCTACAGCGACTGGTATAGC
CCTGCCTGCGGCAAGGCCGGTTGTCAGACCTACAAATGGGAGACATTCCTGACCAGCGAGCTG
CCTCAGTGGCTGTCCGCCAATCGGGCGGTCAAGCCAACCGGCAGCGCCGCTATCGGCCTGAGC
ATGGCCGGCAGCAGCGCCATGATCCTGGCCGCTTATCACCCCCAACAATTTATCTACGCCGGC
TCCCTGAGCGCCCTGCTGGACCCCAGCCAGGGCATGGGACCTAGCCTGATCGGACTTGCTATG
GGCGATGCTGGAGGCTACAAGGCCGCCGACATGTGGGGACCTTCTTCTGATCCTGCCTGGGAG
AGAAACGACCCTACACAGCAGATCCCCAAGCTGGTGGCCAACAATACCAGACTGTGGGTGTAC
TGCGGCAACGGAACACCTAACGAGCTGGGCGGAGCCAACATCCCTGCCGAGTTCCTGGAAAAC
TTCGTGCGGAGCTCTAATCTGAAGTTCCAGGACGCCTACAATGCCGCCGGCGGCCACAACGCC
GTGTTCAACTTCCCACCTAACGGCACCCACAGCTGGGAATACTGGGGCGCTCAGCTGAACGCC
ATGAAAGGCGACCTGCAGTCCTCTCTGGGAGCCGGATGA

In certain embodiments, the fusion protein comprises from N-terminus to C-terminus a Nef^{mut} protein and ESAT-6. In some embodiments, the C-terminus of the Nef^{mut} protein and the N-terminus of ESAT-6 are fused directly without a linker. In particular embodiments, the fusion protein has the amino acid sequence of SEQ ID NO: 87, wherein “X” is V, L or I.

(SEQ ID NO: 87)
MGCKWSKSSVVGWPAVRERMRRAEPAADGVGAASRDLEKHGAITSSNTAATNADCAWLEAQEE
EEVGFPVTPQVPLRPMTYKAAVDLSHFLKEKGGLEGLIHSQRRQDILDLWIYHTQGYFPDWQN
YTPGPGXRYPLTFGWCYKLVPVEPEKLEEANKGENTSLLHPVSLHGMDDPGREVLEWRFDSRL
AFHHVARELHPEYFKNCGPGPTEQQWNFAGIEAAASAIQGNVTSIHSLLDEGKQSLTKLAAAW
GGSGSEAYQGVQQKWDATATELNNALQNLARTISEAGQAMASTEGNVTGMFA

In certain embodiments, the nucleic acid encoding the fusion protein (Nef^{mut}/ESAT-6) is SEQ ID NO: 88, wherein “XXX” is a tri nucleotide sequence that forms a codon that corresponds to valine, leucine or isoleucine. In some embodiments, “XXX” is ATT, ATC, ATA, CTT, CTC, CTA, CTG, TTA, TTG, GTT, GTC, GTA, or is GTG. In certain embodiments, “XXX” is GTT or ATC.

(SEQ ID NO: 88)
ATGGGTTGCAAGTGGTCAAAAAGTAGTGTGGTTGGATGGCCTGCTGTAAGGGAAAGAATGAGA
CGAGCTGAGCCAGCAGCAGATGGGGTGGGAGCAGCATCTCGAGACCTAGAAAAACATGGAGCA
ATCACAAGTAGCAATACAGCAGCTACCAATGOTGATTGTGCCTGGCTAGAAGCACAAGAGGAG
GAGGAGGTGGGTTTTCCAGTCACACCTCAGGTACCTTTAAGACCAATGACTTACAAGGCAGCT
GTAGATCTTAGCCACTTTTTAAAAGAAAAGGGGGGACTGGAAGGGCTAATTCACTCCCAACGA
AGACAAGATATCCTTGATCTGTGGATCTACCACACACAAGGCTACTTCCCTGATTGGCAGAAC
TACACACCAGGACCAGGGXXXAGATATCCACTGACCTTTGGATGGTGCTACAAGCTAGTACCA
GTTGAGCCAGAGAAGTTAGAAGAAGCCAACAAAGGAGAGAACACCAGCTTGTTACACCCTGTG
AGCCTGCATGGAATGGATGACCCGGGGAGAGAAGTGTTAGAGTGGAGGTTTGACAGCCGCCTA
GCATTTCATCACGTGGCCCGAGAGCTGCATCCGGAGTACTTCAAGAACTGCGGACCTGGGCCC
ACCGAGCAGCAATGGAACTTCGCCGGCATCGAGGCCGCTGCTTCCGCCATCCAGGGCAACGTG
ACCAGCATCCACAGCCTGCTGGACGAGGGCAAGCAGAGCCTGACCAAGCTGGCCGCCGCTTGG
GGCGGATCTGGCTCTGAAGCCTACCAGGGCGTGCAGCAGAAATGGGATGCCACAGCCACAGAA
CTGAACAACGCCCTGCAGAACCTGGCTAGAACCATCAGCGAGGCCGGACAGGCCATGGCCAGC
ACCGAAGGCAATGTGACAGGCATGTTCGCCTGA

5.2.2.3. Parasite Antigen

In some embodiments, the immunogenic antigen is a parasite antigen.

In some embodiments, the parasite antigen is a Plasmodium antigen. In various embodiments, the parasite antigen is selected from the group consisting of: Plasmodium falciparum antigen, Plasmodium vivax antigen, Plasmodium ovale antigen, Plasmodium malariae antigen, and Plasmodium knowlesi antigen.

5.2.2.4. Tumor Antigen

In some embodiments, the immunogenic antigen is a tumor antigen.

In certain embodiments, the tumor antigen is an oncofetal antigen. In certain embodiments, the tumor antigen is a tumor-specific antigen. In certain embodiments, the tumor antigen is derived from a protein that is expressed only on cancer cells. In certain embodiments, the tumor antigen is a tumor specific neoantigen.

In certain embodiments, the tumor antigen is a tumor-associated antigen. In certain embodiments, the tumor antigen is derived from a protein that is overexpressed on cancer cells.

5.2.3. Peptide Linker

In various embodiments, the fusion protein disclosed herein further comprises a linker between the exosome-anchoring polypeptide domain and immunogenic antigen polypeptide domain.

In some embodiments, the linker increases the stability of the fusion protein. In some embodiments, the linker increases the bioactivity of the fusion protein. In some embodiments, the linker facilitates the expression of the fusion protein.

In typical embodiments, the linker is a peptide linker. In certain embodiments, the peptide linker is derived from a naturally-occurring multi-domain protein. In certain embodiments, the peptide linker is an empirical linker designed for specific purposes, such as improving structural stability, enhancing bioactivity, increasing expression level, altering the PK profiles, and enabling the in vivo targeting of the fusion protein.

5.2.4. Polynucleotide

In another aspect, provided herein are polynucleotides encoding the fusion proteins described above.

In some embodiments, the polynucleotide is a DNA molecule encoding a fusion protein, wherein the fusion protein comprises from N-terminus to C-terminus, (a) an exosome-anchoring polypeptide domain and (b) an immunogenic antigen polypeptide domain. In typical embodiments, the exosome-anchoring polypeptide domain and immunogenic antigen polypeptide domain are as described above. In certain embodiments, the exosome-anchoring polypeptide is a Nef^{mut} protein. In certain embodiments, the exosome-anchoring polypeptide is a truncated Nef^{mut} protein. In various embodiments, the DNA molecule is codon-optimized.

In some embodiments, the polynucleotide is an RNA molecule encoding a fusion protein, wherein the fusion protein comprises from N-terminus to C-terminus, (a) an exosome-anchoring polypeptide domain and (b) an immunogenic antigen polypeptide domain. In typical embodiments, the exosome-anchoring polypeptide domain and immunogenic antigen polypeptide domain are as described above. In certain embodiments, the exosome-anchoring polypeptide is a Nef^{mut} protein. In certain embodiments, the exosome-anchoring polypeptide is a truncated Nef^{mut} protein. In particular embodiments, the RNA molecule is an mRNA molecule. In certain embodiments, the mRNA molecule has a 5′ cap. In certain embodiments, the mRNA molecule has a 3′ poly-A tail. Where nucleic acid sequences are shown herein, it is understood that for mRNA, the deoxy-thymidines (T) shown are substituted with uridines.

5.2.5. Vector

In another aspect, provided herein are vectors that are capable of expressing the fusion protein as described herein. In various embodiments, the vector comprises at least one polynucleotide, wherein the polynucleotide encodes the fusion protein. In some embodiments, the vector is a plasmid vector. In certain embodiments, the plasmid vector is a pVAX1 vector (Invitrogen). In some embodiments, the vector is a viral vector. In particular embodiments, the viral vector is an adenovirus vector, an adeno-associated virus (AAV) vector, or a vaccinia virus vector.

In some embodiments, the vector is a DNA vector expressing a fusion protein, wherein the fusion protein comprises from N-terminus to C-terminus, (a) an exosome-anchoring polypeptide domain and (b) an immunogenic antigen polypeptide domain. In some embodiments, the vector is an RNA vector expressing a fusion protein, wherein the fusion protein comprises from N-terminus to C-terminus, (a) an exosome-anchoring polypeptide domain and (b) an immunogenic antigen polypeptide domain.

In some embodiments, the vector further comprises one or more of the components selected from: a signal sequence, an origin of replication, one or more marker genes, an enhancer element, a promoter, and a transcription termination sequence. In some embodiments, the vector further comprises a post-transcriptional regulatory element, such as a woodchuck hepatitis virus post-transcriptional regulatory elements (WPRE). In some embodiments, the vector expresses a single immunogenic antigen. In some embodiments, the vector expresses a plurality of immunogenic antigens, such as two, three, four, or five immunogenic antigens. In some embodiments, the vector expresses a plurality of fusion proteins, wherein each fusion protein comprises from N-terminus to C-terminus, (a) an exosome-anchoring polypeptide domain and (b) an immunogenic antigen polypeptide domain. In some embodiments, the vector expresses two, three, four, or five fusion proteins, wherein each fusion protein comprises from N-terminus to C-terminus, (a) an exosome-anchoring polypeptide domain and (b) an immunogenic antigen polypeptide domain.

5.2.6. Extracellular Vesicle

In another aspect, provided herein are extracellular vesicles comprising the fusion protein, the polynucleotide encoding the fusion protein, or the vector expressing the fusion protein.

In some embodiments, the extracellular vesicle is an exosome. In some embodiments, the extracellular vesicle is a nanovesicle.

In some embodiments, the extracellular vesicle comprises a fusion protein, wherein the fusion protein comprises from N-terminus to C-terminus, (a) an exosome-anchoring polypeptide domain and (b) an immunogenic antigen polypeptide domain. In certain embodiments, the exosome-anchoring polypeptide is a Nef^{mut} protein. In certain embodiments, the exosome-anchoring polypeptide is a truncated Nef^{mut} protein. In some embodiments, the Nef^{mut} protein or the truncated Nef^{mut} protein is anchored in the membrane of the extracellular vesicle, and the immunogenic antigen is displayed on the surface of the extracellular vesicle.

In some embodiments, the extracellular vesicle comprises the fusion protein, the polynucleotide encoding the fusion protein, or the vector expressing the fusion protein in the internal space of the extracellular vesicle.

5.2.7. Nanoparticle

In another aspect, provided herein are nanoparticles comprising the fusion protein, the polynucleotide encoding the fusion protein, or the vector expressing the fusion protein.

5.3. Pharmaceutical Composition

In another aspect, provided herein are pharmaceutical compositions comprising the fusion protein, the polynucleotide, the vector, the extracellular vesicle, or the nanoparticle described herein, and a pharmaceutically acceptable excipient.

In some embodiments, the pharmaceutical composition is a vaccine composition. In certain embodiments, the vaccine composition further comprises at least one adjuvant. In particular embodiments, the adjuvant is a saponin adjuvant. In specific embodiments, the saponin adjuvant is Matrix-M™ (Novavax). In particular embodiments, the adjuvant is an alum adjuvant. In specific embodiments, the alum adjuvant is aluminum hydroxide, an aluminum hydroxide gel, or aluminum phosphate. In particular embodiments, the adjuvant is an emulsion adjuvant. In particular embodiments, the adjuvant is a TLR agonist. In specific embodiments, the TLR agonist adjuvant is a CpG oligonucleotide. In some embodiments, the adjuvant is one described in Liang et al., Front. Immunol. 6 Nov. 2020 (doi.org/10.3389/fimmu.2020.589833).

Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, 8th Revised Ed. (2017), incorporated herein by reference in its entirety.

In some embodiments, the pharmaceutical composition comprises a plurality of fusion proteins, such as two, three, four, five, six, seven, eight, nine, or ten fusion proteins, wherein each fusion protein comprises from N-terminus to C-terminus, (a) an exosome-anchoring polypeptide domain and (b) an immunogenic antigen polypeptide domain. In some embodiments, the pharmaceutical composition comprises a plurality of polynucleotides encoding the fusion proteins. In some embodiments, the pharmaceutical composition comprises a plurality of vectors expressing the fusion proteins.

In some embodiments, the pharmaceutical compositions are formulated for administration in single or multiple doses.

5.3.1. Route of Administration

The suitable routes of administration for the pharmaceutical compositions described herein include, but are not limited to, enteral (such as by oral administration), parenteral (such as by subcutaneous, intravenous, intranasal, intramuscular, intradermal, or intrasternal injection or infusion), intranasal, pulmonary (such as by oral inhalation), and topical.

In certain embodiments, the pharmaceutical compositions are formulated for parenteral injection. In particular embodiments, the pharmaceutical composition is in the form of a sterile injectable aqueous or non-aqueous solution or suspension. In some embodiments, the pharmaceutical compositions are formulated for intravenous injection. In some embodiments, the pharmaceutical compositions are formulated for subcutaneous injection.

In some embodiments, the pharmaceutical compositions are formulated for intramuscular injection. In some embodiments, the pharmaceutical composition comprising a vector expressing the fusion protein is formulated for intramuscular injection. In some of these embodiments, the intramuscular injection of the polypeptide or the vector is followed by electroporation. In some embodiments, the electroporation comprises six to eight short pulses (<100 μs) at high field strengths (1000-1300 V/cm). In some embodiments, the electroporation comprises six to eight long pulses (10-20 ms) at low field strengths (200 V/cm). In some embodiments, the electroporation comprises a combination of short high-voltage pulses and long low-voltage pulses.

In certain embodiments, the pharmaceutical compositions are formulated for intranasal administration.

In some embodiments, the pharmaceutical compositions are formulated for topical administration. In particular embodiments, the pharmaceutical compositions are in the form a cream or an ointment.

5.4. Methods of Prevention and Treatment

In another aspect, provided herein are methods of treating or preventing a disease or condition in a subject in need thereof through immunization. Also provided herein are methods of inducing an antigen-specific CD8⁺ cytotoxic T-lymphocyte (CTL) and/or antigen-specific CD4⁺ T cell response in a subject in need thereof. In some embodiments, the method induces an antigen-specific cytotoxic T-lymphocyte (CTL) response in a subject in need thereof. In some embodiments, the method induces an antigen-specific CD4⁺ T cell response in a subject in need thereof. The method comprises administering to the subject an effective amount of the fusion protein, the polynucleotide, the vector, the extracellular vesicle, the nanoparticle, or the pharmaceutical composition disclosed herein.

In some embodiments, the method comprises administering to the subject a single immunogenic antigen. In some embodiments, the method comprises administering to the subject a plurality of immunogenic antigens, such as two, three, four, five, six, seven, eight, nine, or ten immunogenic antigens. In certain embodiments, each of the plurality of antigens is part of a separate fusion protein. In certain embodiments, all of the plurality of antigens are fused to a single exosome-anchoring domain.

In certain embodiments, each of the plurality of antigens is expressed from a separate vector. In certain embodiments, all of the plurality of immunogenic antigens are expressed from the same vector. In embodiments in which the immunogenic antigens are expressed from different vectors, the plurality of immunogenic antigens are administered simultaneously to the subject. In some other embodiments, the immunogenic antigens are administered sequentially to the subject.

In certain embodiments, the method comprises administering to the subject a combination of two immunogenic antigens. In certain embodiments, the method comprises administering to the subject a combination of three immunogenic antigens. In certain embodiments, the method comprises administering to the subject a combination of four immunogenic antigens. In certain embodiments, the method comprises administering to the subject a combination of five immunogenic antigens.

In some embodiments, the disease or condition is an infection and the subject has or is at risk for an infection. The infection can be caused by various infectious agents, such as viruses, bacteria, fungi, or parasites. In certain embodiments, the disease or condition is a viral infection, and the subject has or is at risk for a viral infection. In various embodiments, the viral infection is selected from a human papillomavirus (HPV) infection, a human immunodeficiency virus (HIV) infection, a hepatitis B virus (HBV) infection, a hepatitis C virus (HCV) infection, an Ebola virus infection, a West Nile virus infection, a Crimean-Congo virus infection, a dengue virus infection, and an influenza virus infection. In certain embodiments, the disease or condition is a bacterial infection, and the subject has or is at risk for a bacterial infection. In some embodiments, the bacterial infection is a Mycobacterium tuberculosis infection, and the subject has or is at risk for tuberculosis (TB). In certain embodiments, the disease or condition is a parasitic infection, and the subject has or is at risk for a parasitic infection. In some embodiments, the parasitic infection is a Plasmodium infection, and the subject has or is at risk for malaria.

In some embodiments, the disease or condition is cancer, and the subject has or is at risk for cancer. In certain embodiments, the disease or condition is cervical cancer, and the subject has or is at risk for cervical cancer. In certain embodiments, the disease or condition is head and neck cancer, and the subject has or is at risk for head and neck cancer. In certain embodiments, the disease or condition is liver cancer, and the subject has or is at risk for liver cancer.

5.4.1. HPV Infection

In some embodiments, the disease or condition is a human papillomavirus (HPV) infection. In some embodiments, the method induces a cytotoxic T-lymphocyte (CTL) and/or antigen-specific CD4⁺ T cell response in a patient who has an HPV infection. In particular embodiments, the method induces a cytotoxic T-lymphocyte (CTL) response in a patient who has an HPV infection. In some embodiments, the method induces a cytotoxic T-lymphocyte (CTL) and/or antigen-specific CD4⁺ T cell response in a patient who is at risk for HPV infection. In certain embodiments, the method induces a cytotoxic T-lymphocyte (CTL) response in a patient who is at risk for HPV infection.

In some embodiments, the method comprises administering to a patient who has an HPV infection an effective amount of the fusion protein, the polynucleotide, the vector, the extracellular vesicle, the nanoparticle, or the pharmaceutical composition described herein, wherein the immunogenic antigen polypeptide domain is an immunogenic antigen from human papilloma virus (HPV). In some embodiments, the method comprises administering to a patient who is at risk for HPV infection an effective amount of the fusion protein, the polynucleotide, the vector, the extracellular vesicle, the nanoparticle, or the pharmaceutical composition, wherein the immunogenic antigen polypeptide domain is an immunogenic antigen from HPV.

In some embodiments, a fusion protein comprising a Nef^{mut} protein and the E6 antigen of HPV16 is administered to the patient. In some embodiments, a fusion protein comprising a Nef^{mut} protein and the E7 antigen of HPV16 is administered to the patient. In certain embodiments, a fusion protein comprising a Nef^{mut} protein and the E6 antigen of HPV16 and a fusion protein comprising a Nef^{mut} protein and the E7 antigen of HPV16 are administered to the patient. In some embodiments, a fusion protein comprising a truncated Nef^{mut} protein and the E6 antigen of HPV16 is administered to the patient. In some embodiments, a fusion protein comprising a truncated Nef^{mut} protein and the E7 antigen of HPV16 is administered to the patient. In certain embodiments, a fusion protein comprising a truncated Nef^{mut} protein and the E6 antigen of HPV16 and a fusion protein comprising a truncated Nef^{mut} protein and the E7 antigen of HPV16 are administered to the patient. In some of these embodiments, the E6 antigen and/or the E7 antigen are detoxified.

In some embodiments, a polynucleotide encoding the fusion protein comprising a Nef^{mut} protein and the E6 antigen of HPV16 is administered to the patient. In some embodiments, a polynucleotide encoding the fusion protein comprising a Nef^{mut} protein and the E7 antigen of HPV16 is administered to the patient. In certain embodiments, a polynucleotide encoding the fusion protein comprising a Nef^{mut} protein and the E6 antigen of HPV16 and a polynucleotide encoding the fusion protein comprising a Nef^{mut} protein and the E7 antigen of HPV16 are administered to the patient. In some embodiments, a polynucleotide encoding the fusion protein comprising a truncated Nef^{mut} protein and the E6 antigen of HPV16 is administered to the patient. In some embodiments, a polynucleotide encoding the fusion protein comprising a truncated Nef^{mut} protein and the E7 antigen of HPV16 is administered to the patient. In certain embodiments, a polynucleotide encoding the fusion protein comprising a truncated Nef^{mut} protein and the E6 antigen of HPV16 and a polynucleotide encoding the fusion protein comprising a truncated Nef^{mut} protein and the E7 antigen of HPV16 are administered to the patient. In some of these embodiments, the E6 antigen and/or the E7 antigen are detoxified. In certain embodiments, the nucleic acid encoding the E6 antigen and/or the E7 antigen is codon optimized.

In some embodiments, a vector expressing the fusion protein comprising a Nef^{mut} protein and the E6 antigen of HPV16 is administered to the patient. In some embodiments, a vector expressing the fusion protein comprising a Nef^{mut} protein and the E7 antigen of HPV16 is administered to the patient. In certain embodiments, a vector expressing the fusion protein comprising a Nef^{mut} protein and the E6 antigen of HPV16 and a vector expressing the fusion protein comprising a Nef^{mut} protein and the E7 antigen of HPV16 are administered to the patient. In some embodiments, a vector expressing the fusion protein comprising a truncated Nef^{mut} protein and the E6 antigen of IPV16 is administered to the patient. In some embodiments, a vector expressing the fusion protein comprising a truncated Nef^{mut} protein and the E7 antigen of IPV16 is administered to the patient. In certain embodiments, a vector expressing the fusion protein comprising a truncated Nef^{mut} protein and the E6 antigen of HPV16 and a vector expressing the fusion protein comprising a truncated Nef^{mut} protein and the E7 antigen of IPV16 are administered to the patient. In some embodiments, the E6 and E7 antigens of HPV16 are expressed from the same vector. In some other embodiments, the E6 and E7 antigens of HPV16 are expressed from different vectors. In certain embodiments, the E6 antigen and/or the E7 antigen are detoxified. In certain embodiments, the nucleic acid encoding the E6 antigen and/or the E7 antigen is codon optimized.

5.4.2. Tuberculosis Infection

In some embodiments, the disease or condition is a human tuberculosis (TB) infection. In some embodiments, the method induces a cytotoxic T-lymphocyte (CTL) and/or antigen-specific CD4⁺ T cell response in a patient who has a latent tuberculosis infection (LTBI). In particular embodiments, the method induces a cytotoxic T-lymphocyte (CTL) response in a patient who has a LTBI. In some embodiments, the method induces a cytotoxic T-lymphocyte (CTL) and/or antigen-specific CD4⁺ T cell response in a patient who has a TB infection. In certain embodiments, the method induces a cytotoxic T-lymphocyte (CTL) response in a patient who has a TB infection. In some embodiments, the method induces a cytotoxic T-lymphocyte (CTL) and/or antigen-specific CD4⁺ T cell response in a patient who is at risk for TB infection. In certain embodiments, the method induces a cytotoxic T-lymphocyte (CTL) response in a patient who is at risk for TB infection.

In some embodiments, a vector expressing the fusion protein comprising a Nef^{mut} protein and the antigen 85B (Ag85B) of Mycobacterium tuberculosis is administered to the patient. In some embodiments, a vector expressing the fusion protein comprising a Nef^{mut} protein and the Early secretory antigenic target-6 (ESAT-6) of Mycobacterium tuberculosis is administered to the patient. In certain embodiments, a vector expressing the fusion protein comprising a Nef^{mut} protein and the Ag85B antigen of Mycobacterium tuberculosis and a vector expressing the fusion protein comprising a Nef^{mut} protein and the ESAT-6 antigen of Mycobacterium tuberculosis are administered to the patient. In some embodiments, a vector expressing the fusion protein comprising a truncated Nef^{mut} protein and the Ag85B antigen of Mycobacterium tuberculosis is administered to the patient. In some embodiments, a vector expressing the fusion protein comprising a truncated Nef^{mut} protein and the ESAT-6 antigen of Mycobacterium tuberculosis is administered to the patient. In certain embodiments, a vector expressing the fusion protein comprising a truncated Nef^{mut} protein and the Ag85B antigen of Mycobacterium tuberculosis and a vector expressing the fusion protein comprising a truncated Nef^{mut} protein and the ESAT-6 antigen of Mycobacterium tuberculosis are administered to the patient. In certain embodiments, the nucleic acid encoding the Ag85B antigen and/or the ESAT-6 antigen is codon optimized. In certain embodiments, the N-terminal signal peptide of Ag85B is removed.

### 6. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

6.1. General Methods

### Vectors

DNA vectors expressing wild type (wt) Nef and Nef^{mut} have previously been described in D'Aloja P, et al., 2001, J Gen Virol 82:2735-2745. DNA vectors expressing Nef^{mut}/E6 (E6 sequence: AF486315.1, www.ncbi.nlm.nih.gov) have previously been described in Di Bonito P, et al., 2015, Viruses 7:1079-1099. DNA vectors expressing Nefmut/E7 have previously been described in Di Bonita P, et al., 2017, Int. J. Nanomedicine 12:4579-4591. Each of these references is incorporated herein by reference in its entirety.

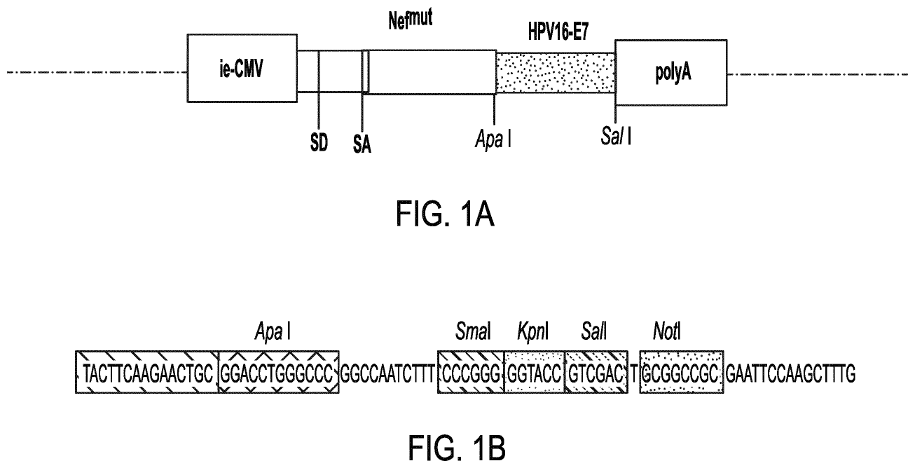

The pTarget-Nef^{mut}/E7 vector was obtained as follows: the backbone was pTarget cloning vector (Promega) where the full length Nef^{mut} sequence was inserted between the two T overhangs at the polylinker region. At the 3′ end of the Nef^{mut} open reading frame (ORF), the stop codon was replaced by a GPGP (SEQ ID NO: 55) linker including an Apa I site at the 3′ end in a way that the ligation with downstream heterologous sequences digested by Apa I resulted in a unique, in frame sequence (FIGS. 1A and 1). This vector was referred to as pTarget-Nef^{mut}/fusion. Downstream restriction sites from the vector polylinker were useful for the directional insertion of foreign sequences. HPV16 E7 ORF was amplified from a vector harboring the sequence AF 486326.1 (www.ncbi.nlm.nih.gov) by PCR with Platinum High Fidelity Taq polymerase (Invitrogen) using the forward primer: TATAGGGCCCCATGGAGATACACC (SEQ ID NO: 56) and the reverse primer: TATCGTCGACTTATGGTTCTGGGAACA (SEQ ID NO: 57). The PCR product was then Apa I/SalI digested and inserted in the appropriately digested pTarget-Nef^{mut}/fusion vector.

pTarget-Nef^{mut}PL was obtained by digesting the vector pTarget-Nef^{mut}/fusion, i.e., a pTarget vector (Invitrogen) where the whole Nef^{mut} sequence was inserted between the two T overhangs of the polylinker region. At the 3′ end of the Nef^{mut} open reading frame (ORF), the stop codon was replaced by a GPGP (SEQ ID NO: 55) linker including an Apa I site at this 3′ end in a way that the ligation with downstream heterologous sequences digested by Apa I results in a unique, in frame sequence. The pTarget-Nef^{mut}/fusion was digested with the Sma I enzyme, recognizing a first restriction site just downstream to the most C-terminal typical Nef^{mut} mutation (i.e., ^{E}177^{G}), and a second one at the 3′ terminal vector polylinker. The subsequent re-ligation generated a C-terminal 29 amino acid deletion, with the de novo formation of a stop codon just downstream the Sma I restriction site.

For pTarget-Nef^{mut}/E6 detoxified (E6^{DETOX}), the HPV16 E6 ORF was excised from the vector pTarget-Nef^{mut}/E6 by Apa 1/Sal I digestion, and replaced with a “detoxified” E6 ORF where the ^{G}130^{V} amino acid substitution generated a loss-of-function of E6 by hindering the E6 interaction with p53. See Shamanin V A, et al., 2008, J Virol 82:3912-3920, which is incorporated by reference in its entirety.

The cloning strategy of pTarget-Nef^{mut}/E6 optimized-detoxified (E6^{OD}) was similar to that followed to obtain the pTarget-Nef^{mut}/E6^{DETOX}. The E6 ORF was detoxified by the ^{G}130^{V} amino acid substitution. In addition, the whole E6 ORF was codon optimized through an adhoc algorithm provided by Codon Optimization On-Line (COOL) service (cool.syncti.org), which introduced 134 base substitutions.

For pTarget-Nef^{mut}PL/E6^{DETOX}, the Nef^{mut}PL ORF was PCR amplified using a reverse primer with an Apa I site in a way that, as in the pTarget-Nef^{mut}/fusion vector, the Apa I insertion of downstream ORFs results in an in-frame sequence, and thus in a fusion protein. The resulting vector was referred to as Nef^{mut}PL/fusion. The cloning and synthesis strategy to obtain the pTarget-Nef^{mut}PL/E6^{DETOX} vector was identical to that described for the pTarget-Nef^{mut}/E6^{DETOX} vector, except that the E6^{DETOX} ORF was inserted in the pTarget-Nef^{mut}PL/fusion vector.

The cloning and synthesis strategy of pTarget-Nef^{mut}PL/E6^{OD} was identical to that described for the pTarget-Nef^{mut}/E6^{DETOX} vector, except that the E6^{OD} ORF was inserted in the pTarget-Nef^{mut}PL/fusion vector.

For pTarget-Nef^{mut}/E7 detoxified (E7^{DETOX}) the HPV16 E7 ORF was excised from the vector pTarget-Nef^{mut}/E7 by Apa I/Sal I digestion, and replaced with a “detoxified” E7 ORF. This E7 variant was obtained by inserting three amino acid substitutions, namely three glycines, at positions 21, 24, and 26 within the retinoblastoma protein (pRB) binding site. In this way, the E7-specific immortalizing activity was reduce or abrogated. See Smahel M, et al., 2001, Virology 281:231-238, which is incorporated by reference in its entirety.

The cloning strategy of pTarget-Nef^{mut}/E7 optimized-detoxified (E7^{OD}) was similar to that followed to obtain the pTarget-Nef^{mut}/E7^{DETOX}. The E7 ORF was detoxified. Additionally, the whole E7 ORF was codon optimized in line with the published results from Cid-Arregui and colleagues (see Cid-Arregui A, et al., 2003, J Virol 77:4928-4937, which is incorporated by reference in its entirety) through the introduction of 64 base substitutions.

For pTarget-Nef^{mut}PL/E7^{DETOX}, the pTarget-Nef^{mut}PL/fusion vector was excised at Apa I and Sal I restriction sites where the E7^{DETOX} ORF as described above, was inserted in frame.

For pTarget-Nef^{mut}PL/E7^{OD}, the pTarget-Nef^{mut}PL/fusion vector was excised at Apa I and Sal I restriction sites where the E7^{OD} ORF as described above, was inserted in frame.

For pcDNA3.1-E6^{OD}, the E6^{OD} ORF described above, but including both Kozak sequences and the ATG start codon at the 5′ end, was inserted in the Not I and Apa I sites of the pcDNA3.1 vector (Invitrogen) polylinker. A 6×His tag sequence (i.e., 5′ CACCATCACCATCACCAT 3′(SEQ ID NO: 58) was included at the 3′ end just before the stop codon.

For pcDNA3.1-E7^{OD}, the E7^{OD} ORF described above, but including both Kozak sequences and the ATG start codon at the 5′ end, was inserted in the Not I and Apa I sites of the pcDNA3.1 vector (Invitrogen) polylinker. A 6×His tag sequence (i.e., 5′ CACCATCACCATCACCAT 3′(SEQ ID NO: 58) was included at the 3′ end just before the stop codon.

### Analysis of Fusion Protein Expression

HEK293T cells were transiently transfected with the DNA vectors described above. 5×10⁶ cells were seeded in 10 cm Petri dishes in 10% FCS DMEM. After 24 hours, cell transfections were carried out using 3 μg/ml polyethylenimine (PEI) (Sigma, cat n. 408727) and 5 μg DNA in 2% FCS DMEM.

After additional 24 hours, extracellular vesicle (EV)-depleted medium (i.e., DMEM supplemented with 5% FCS previously EV-depleted by ultracentrifugation for 4 hours at 70,000 g, 4° C.) was added. At the assay completion (i.e., 48-72 after transfection), both cells and supernatants were harvested.

Western blot analysis on cell lysates was performed by washing cells twice with 1×PBS (pH 7.4) and lysing them for 20 min on ice with lysis buffer (20 mM HEPES pH 7.9, 50 mM NaCl, 10 mM EDTA, 2 mM EGTA, 0.5% nonionic detergent IGEPAL CA-630, 0.5 mM dithiothreitol, 20 mM sodium molybdate, 10 mM sodium orthovanadate, 100 mM sodium fluoride, 10 μg/mL leupeptin, 0.5 mM phenylmethylsulfonyl fluoride). Whole cell lysates were centrifuged at 6,000×g for 10 min at 4° C. The protein concentration of cell extracts was determined by the Lowry protein quantitation assay. Aliquots of cell extracts containing 30 to 50 μg of total proteins were resolved by 8-12% sodium dodecyl sulfate-polyacrylamide gel electrophoresis (SDS-PAGE) and transferred by electroblotting on a 0.45 M pore size nitrocellulose membrane (Amersham) overnight using a Bio-Rad Trans-Blot. For immunoassays, membranes were blocked with 5% non-fat dry milk in PBS containing 0.1% Triton X-100 for 1 h at room temperature, then incubated overnight at 4° C. with specific antibodies diluted in PBS containing 0.1% Triton X-100.

For exosome isolation, supernatants from transfected cells underwent differential centrifugations including a first ultracentrifugation at 10,000×g for 30 min. Supernatants were then harvested, filtered with 0.22 μM pore size, and ultracentrifuged at 70,000 g for 2 hours. Pelleted vesicles were resuspended in PBS, and ultracentrifuged again at 70,000 g for 1 h. Finally, exosomes were lysed in PBS containing 0.1% Triton X-100 for western blot analysis. See Thery C., et al., 2006, Curr Prot Cell Biol. doi: 10.1002/0471143030.cb0322s30, incorporated by reference in its entirety.

The antibodies used in immunoblots were: sheep anti-Nef antiserum ARP 444 (a generous gift of M. Harris, University of Leeds, Leeds, UK, for unrestricted use), anti-His-tag (BioRad MCA1396GA), anti-ALIX (Santa Cruz SC9901), anti-3-Actin (Cell Signaling 5125S) and the appropriate HRP-conjugated secondary Abs: anti-sheep (Santa Cruz SC2770), anti-mouse (BIORAD 170-6516), anti-rabbit (Amersham NA934). ECL (Thermo Scientific 34580) revelation was carried out with a ChemiDoc apparatus (BioRad).

### In Vivo Study

Six-week old C57BL/6 female mice were obtained from Charles River. The day before the first inoculation, microchips from DATAMARS were inserted subcutaneously (s.c.) at the back of the neck between the shoulder blades on the dorsal midline. The indicated amounts of both control and fusion protein expression vectors were diluted in sterile 0.9% saline solution. As control vector, the pTarget homologous pcDNA3.1 (Invitrogen) was used.

Both quality and quantity of the DNA preparation were checked by 260/280 nm absorbance and electrophoresis assays. Each inoculum volume was measured by micropipette, loaded singly into a 1 mL syringe without dead volume, and injected into mouse quadriceps.

For the electroporation procedure, mice were anesthetized with isoflurane. Immediately after inoculation, electroporation was applied at the site of injection through the Agilpulse BTX device using a 4-needle array 4 mm gap, 5 mm needle length (BTX, cat n. 15497370) with the following parameters:
1 pulse of 450 V for 50 μsec;
0.2 msec interval;
1 pulse of 450 V for 50 μsec;
50 msec interval;
8 pulses of 110 V for 10 msec with 20 msec intervals.

EP parameters were those described in the literature (Hobernik D, et al., 2018, Int. J. Med Sci 19: e3605, incorporated by reference in its entirety) and recommended by the manufacturer.

Spleens were explanted and placed into a 2 mL Eppendorf tubes filled with 1 mL of RPMI 1640 (Gibco), 50 μM 2-mercaptoethanol (Sigma). Spleens were transferred into a 60 mm Petri dish containing 2 mL of RPMI 1640 (Gibco), 50 μM 2-mercaptoethanol (Sigma). Splenocytes were extracted by incising the spleen with sterile scissors and pressing the cells out of the spleen sac with the plunger seal of a 1 mL syringe. After addition of 2 mL of RPMI medium, cells were transferred into a 15 mL conical tube, and the Petri plate was washed with 4 mL of medium to collect the remaining cells. After a three-minute sedimentation, splenocytes were transferred to a new sterile tube to remove cell and tissue debris. Counts of live cells were carried out by the trypan blue exclusion method. A total of 5×10⁶ fresh splenocytes was resuspended in RPMI complete medium, containing 50 μM 2-mercaptoethanol and 10% FBS, and tested by IFN-γ ELISpot assay. Leftover cells were frozen in aliquots of 20-30×10⁶ cells/mL in 90% FBS (Gibco), 10% DMSO (Sigma).

### Syngeneic Mouse Model

TC-1 cells used to generate the syngeneic mouse model were derived from a tumor implanted s.c. in a C57BL/6 female mouse. Explanted TC-1 cells were assumed to have an optimal tumorigenicity. They were expanded, and multiple stocks were frozen after no more than five passages to preserve their tumorigenicity. In the present experiments, the cells were characterized by qRT-PCR analysis for the HPV16 E6 and E7 expression before implantation. The assay was carried out on total RNA extracted from one million of either TC-1 or, as negative control, murine macrophage RAW 264.7 cells (ATCC, TIB71), with the TRIzol Reagent (Invitrogen) following the manufacturer's recommendations. One g of the RNA was used to synthesize cDNA by employing the Reverse Transcription (RT) System kit (Promega). One aliquot (2 l) of cDNA was amplified using the oligonucleotide primers from the E6 sequence (forward: 5′-AATGTTTCAGGACCCACAGG-3′ (SEQ ID NO: 59), and reverse: 5′-TTGTTTGCAGCTCTGTGCAT-3′ (SEQ ID NO: 60) or from the E7 sequence (forward: 5′-CAAGTGTGACTCTACGCTTCGG-3′ (SEQ ID NO: 61), and reverse: 5′-GTGGCCCATTAACAGGTCTTCCAA-3′ (SEQ ID NO: 62). Genomic DNA contamination was checked by including RT (−) controls, i.e., conditions run in the absence of RT. The RT reaction was normalized by amplifying samples also for hypoxanthine guanine phosphoribosyltransferase (HPRT) as house-keeping gene. RT-PCR was performed by means of the SYBR Green RT-PCR kit (Qiagen) and the Applied Biosystems 7500 Real-Time PCR System (Applied Biosystems). The reaction mix for each PCR sample comprised: 12.5 μl of SYBR Green mix, 8.5 μl of distilled water, 2 μl of cDNA, 1 μl of primer mix (20 nM of each primer). The PCR reactions were led at 95° C. for 15″, 60° C. for 30″, 72° C. for 1′, for 40 cycles. Data were collected during every elongation step (72° C.) and during final ramping (to control specificity), and analyzed by the Applied Biosystems 7500 SDS software (Applied Biosystems, Carlsbad, Calif.) using the 2^{-DDCt} method.

C57BL/6 mice (12 per group) were challenged with 2×10⁵ TC-1 cells and, the day after the tumor appearance, i.e., when tumor masses became detectable by palpation, the indicated amounts of each expression vector were injected into mouse quadriceps followed by electroporation.

To collect peripheral blood mononuclear cells (PBMCs) for evaluating the immune responses, seven days after the second immunization, 200 μL of blood were collected from each mouse through retro orbital bleeding. Tumor growth was monitored daily by visual inspection, palpation, and measurement of diameter by an electronic caliper, and calculated as (length×width²)/2. Mice were sacrificed by cervical dislocation if in poor health or as soon as tumors reached the size of 1 cm³. After incubation with ACK lysing buffer (Gibco) to eliminate red blood cells, the IFN-γ ELISpot assay was carried out by seeding 0.5-2×10⁵ live cells (as assessed by the trypan blue exclusion method) in each microwell.

### IFN-γ ELISpot Assay

2.5×10⁵ live splenocytes or 0.5-2×10⁵ PBMCs were seeded in each microwell. Cultures were run in triplicate in ELISpot multiwell plates (Millipore, cat n. MSPS4510) pre-coated with the AN18 mAb against mouse IFN-γ (Mabtech) in RPMI 1640 (Gibco) plus 10% FBS (Gibco) for 16 h in the presence or absence of 5 μg/mL of the following peptides:

E6 (for CD4⁺ T lymphocytes)
68-83:
(SEQ ID NO: 63)
CIVYRDGNPYAVCDKC
96-110:
(SEQ ID NO: 64)
EQQYNKPLCDLLIRC;
E6 (for CD8⁺ T lymphocytes)
18-26:
(SEQ ID NO: 65)
KLPQLCTEL;
50-57:
(SEQ ID NO: 66)
YDFAFRDL;
109-117:
(SEQ ID NO: 67)
RCINCQKPL;
127-135:
(SEQ ID NO: 68)
DKKQRFMNI.
E7 (for CD4⁺ T lymphocytes):
44-60:
(SEQ ID NO: 69)
QAEPDRAHYNIVTFCCK;
E7 (for CD8⁺ T lymphocytes):
21-28:
(SEQ ID NO: 70)
DLYCYEQL;
49-57:
(SEQ ID NO: 71)
RAHYNIVTF;
67-75:
(SEQ ID NO: 72)
LCVQSTHVD.

See Tindle R W, et al., 1991, PNAS 88:5887-5891; Bauer S, et al., 1995, Scand J. Immunol 42:317-323; de Oliveira L M, et al., 2015, PLoSOne doi.org/10.1371/journal.pone.0138686.g001; and Azoury-Ziadeh R, et al., 1999, Viral Immunol 12: 297-312; each of which is incorporated by reference in its entirety.

As a negative control, 5 μg/mL of the H2-K^{b}-binding HCV-NS3 specific peptide ITQMYTNV (SEQ ID NO: 73) (see Mikkelsen M, et al., 2011, J. Immunol 186:2355-2364, incorporated by reference in its entirety) were used. More than 70% pure preparations of the peptides were obtained from either UFPeptides, Ferrara, Italy, or JPT, Berlin, Germany. For cell activation control, cultures were treated with 10 ng/mL PMA (Sigma) plus 500 ng/mL of ionomycin (Sigma). After 16 hours, cultures were removed, and the wells were incubated with 100 μL of 1 μg/ml of the R4-6A2 biotinylated anti-IFN-γ (Mabtech) for 2 hours at room temperature. Wells were then washed and treated for 1 hour at room temperature with 1:1000 diluted streptavidine-ALP preparations from Mabtech. After washing, spots were developed by adding 100 μL/well of SigmaFast BCIP/NBT, cat. n. B5655. The spot-forming cells were finally analyzed and counted using an AELVIS ELISpot reader.

### Intracellular Cytokine Staining (ICS) and Flow Cytometry Assay

Thawed splenocytes were seeded (2×10⁶ live cells per well) in 48-well plates in RPMI medium, 10% FCS, 50 μM 2-mercaptoethanol (Sigma), and 1 μg/mL Brefeldin A (BD Biosciences) for 16 hours at 37° C. Control conditions for cell activation were carried out by adding 10 ng/ml PMA (Sigma) and 1 μg/mL ionomycin (Sigma). To assay HPV16 E6- and E7-CD8⁺ T cell specific activation, 5 μg/mL of the 9-mer peptides described above binding the H2-K^{b} complex of C57BL/6 mice were added. As negative control, 5 μg/ml of the H2-K^{b}-binding HCV-NS3 specific peptide were used.

After 16 hours, cultures were stained with 1 μl of LIVE/DEAD Fixable Aqua Dead Cell reagent (Invitrogen ThermoFisher) in 1 mL of PBS for 30 minutes at 4° C. and washed twice with 500 μl of PBS. To minimize nonspecific staining, cells were pre-incubated with 0.5 μg of Fc blocking mAbs (i.e., anti-CD16/CD32 antibodies, Invitrogen/eBioscience) in 100 μL of PBS with 2% FBS for 15 minutes at 4° C.

All mAb batches were preventively tested to establish the optimal concentrations to be used in ICS assays on splenocytes. For the detection of cell surface markers, cells were stained with 2 μL of the following Abs: FITC conjugated anti-mouse CD3, APC-Cy7 conjugated anti-mouse CD8a, and PerCP conjugated anti-mouse CD4 (BD Biosciences) and incubated for 1 hour at 4° C.

After washing, cells were permeabilized and fixed through the Cytofix/Cytoperm kit (BD Biosciences) as per the manufacturer's recommendations, and stained for 1 hour at 4° C. with 2 μl of the following Abs: PE-Cy7 conjugated anti-mouse IFN-γ, PE conjugated anti-mouse IL-2 (Invitrogen eBioscience), and BV421 rat anti-mouse TNF-α BD Biosciences in a total of 100 μL of 1× Perm/Wash Buffer (BD Biosciences). After two washes, cells were fixed in 200 μL of 1×PBS/formaldehyde (2% v/v). Samples were then assessed by a Gallios flow cytometer and analyzed using Kaluza software (Beckman Coulter).

Gating strategy was as follows: live cells as detected by Aqua LIVE/DEAD Dye vs. FSC-A, singlet cells from FSC-A vs. FSC-H (singlet 1) and SSC-A vs SSC-W (singlet 2), CD3 positive cells from CD3 (FITC) vs. SSC-A, CD8 or CD4 positive cells from CD8 (APC-Cy7) vs. CD4 (PerCP). The CD8⁺ cell population was gated against APC-Cy7, PE, and BV421 to observe changes in IFN-γ, IL-2, and TNF-α production, respectively. Boolean gates were created in order to determine any cytokine co-expression pattern.

### CTL Assay

CD8⁺ T cells were isolated from splenocytes by positive immunomagnetic selection (Miltenyi Biotec Gmbh, Teterow, Germany). They were put in co-culture for 4 hours in RPMI 10% FCS with EL-4 cells (ATCC TIB-39) previously labeled with carboxyfluorescein succinimidyl ester (CFSE, Invitrogen, Thermo Fisher) following the manufacturer's recommendation, and treated overnight with either E7 or unrelated peptides. The co-cultures were run at 10:1 effector/target cell ratio in 200 μl of RPMI 10% in U-bottom 96 well plates. Afterwards, EL-4 cell mortality was scored by FACS analysis soon after addition of 7-AAD (Sigma) at final concentration of 1 μg/ml.

### 6.2. Example 1: DNA Dose-Response after Intramuscular Administration or Intramuscular+Electroporation Administration

The HPV16 E7-specific CD8⁺ T cell immune responses elicited by the intramuscular (i.m.) injection of Nef^{mut}/E7-expressing DNA vector with or without electroporation (EP) procedures were compared. The experimental design is shown in Table 2 below.

TABLE 2

| Group | Inoculum |
|---|---|
| A | i.m. |
| | pcDNA 3.1 empty vector |
| | 10 μg, 100 μL |
| B | i.m. |
| | pTarget-Nef^{mut}/E7 |
| | 10 μg, 100 μL |
| C | i.m. |
| | pTarget-Nef^{mut}/E7 |
| | 25 μg, 100 μL |
| D | i.m. |
| | pTarget-Nef^{mut}/E7 |
| | 50 μg, 100 μL |
| E | i.m. + EP |
| | pcDNA 3.1 empty vector |
| | 30 μL |
| F | i.m. + EP |
| | pTarget-Nef^{mut}/E7 |
| | 1 μg, 30 μL |
| G | i.m. + EP |
| | pTarget-Nef^{mut}/E7 |
| | 5 μg, 30 μL |
| H | i.m. + EP |
| | pTarget-Nef^{mut}/E7 |
| | 10 μg, 30 μL |

All groups included 3 mice, except A and E control groups containing 2 mice. The above indicated doses of DNA were injected in each quadriceps, which were the sites where the electric field was applied in the EP conditions. The injections were repeated 14 days thereafter.

The immunization efficiencies were evaluated by HIPV16 E7-specific CD4⁺ and CD8⁺ T lymphocyte activation in IFN-γ ELISpot assays carried out with splenocytes isolated from mice injected with the Nef^{mut}/E7-expressing DNA vector.

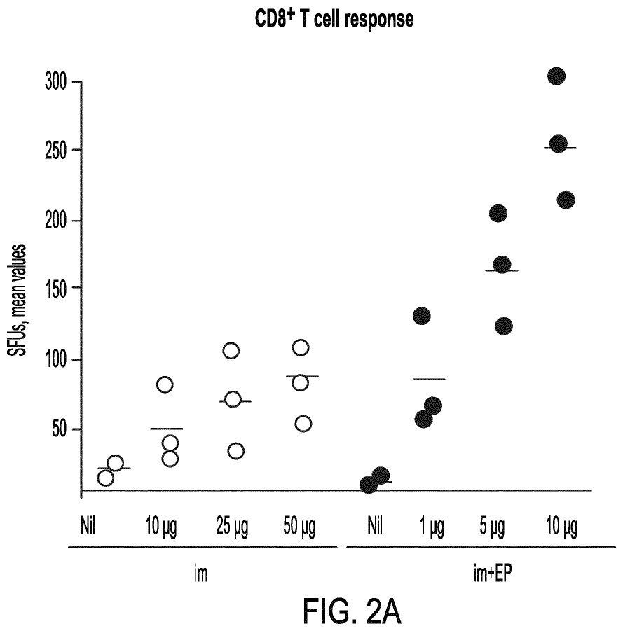

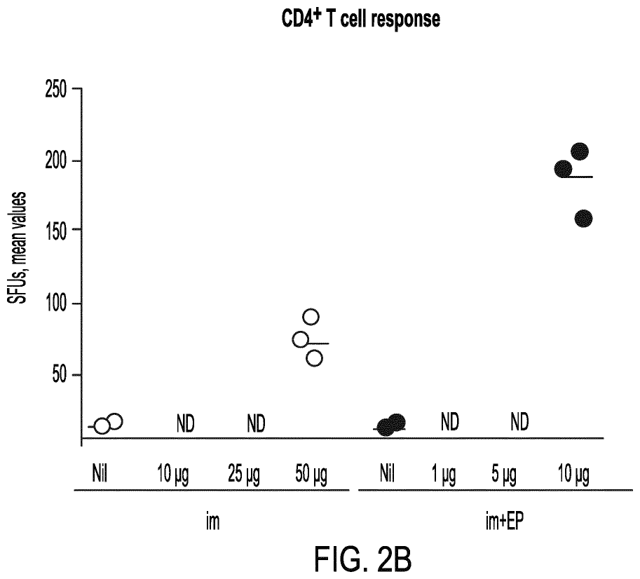

The HPV16 E7-specific CD8⁺ and CD4⁺ T cell immunity induced in mice by injection of Nef^{mut}/E7 expressing DNA vector in the absence or presence of EP are shown FIGS. 2A and 2B. When DNA injections were followed by EP, more potent E7-specific CD8⁺ and CD4⁺ T immunities were produced. In particular, in mice injected with 10 μg of DNA, the subsequent EP led to a 5-fold increase of the immunogenicity compared to i.m. injection alone using the highest DNA dose, i.e., 50 μg. Notably, this dose appeared less immunogenic than the dose of 5 μg given by i.m. injection+EP.

Based on these results, i.m. injection of 10 μg of DNA followed by EP was the dose chosen for the subsequent examples.

### 6.3. Example 2: DNA Optimization

DNA vectors were generated to test the effect of detoxification and codon optimization. For detoxification, E6 and E7 were detoxified to reduce or abrogate binding to the host-cell proteins, tumor suppressor genes p53 and pRb, respectively. Additionally, EV uploading and immunogenicity of a C-terminus truncated Nef^{mut} (referred to as Nef^{mut}PL) have been tested. The experimental design is shown in Table 3 below.

TABLE 3

| Group | Inoculum |
|---|---|
| A | i.m. + EP |
| | pcDNA3.1 empty vector |
| | 10 μg, 30 μL |
| B | i.m. + EP |
| | pTarget-Nef^{mut}/E7 |
| | 10 μg, 30 μL |
| C | i.m. + EP |
| | pTarget-Nef^{mut}/E7^{OD} |
| | 10 μg, 30 μL |
| D | i.m. + EP |
| | pTarget-Nef^{mut}PL/E7^{OD} |
| | 10 μg, 30 μL |
| E | i.m. + EP |
| | pTarget-Nef^{mut}/E6 |
| | 10 μg, 30 μL |
| F | i.m. + EP |
| | pTarget-Nef^{mut}/E6^{OD} |
| | 10 μg, 30 μL |
| G | i.m. + EP |
| | pTarget-Nef^{mut}PL/E6^{OD} |
| | 10 μg, 30 μL |

All groups included 3 mice, except control group (A) comprising 2 mice. Inoculations were carried out in each quadriceps and electroporation was performed after each DNA injection. A second identical immunization was performed 14 days later.

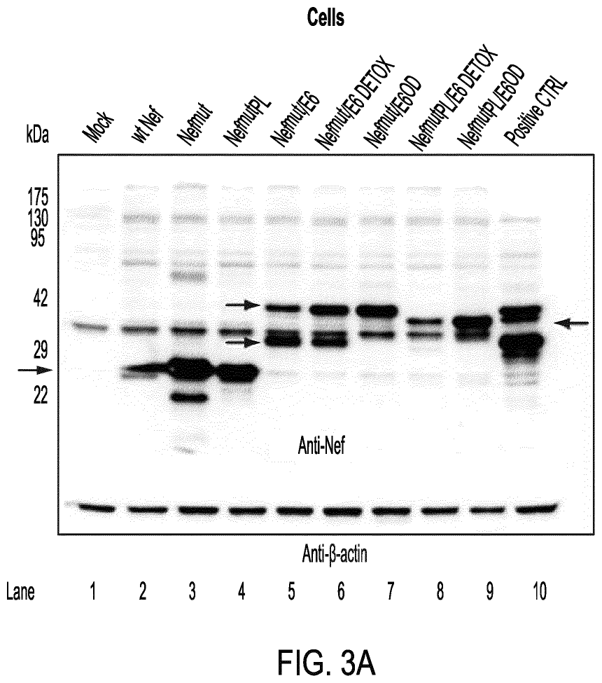

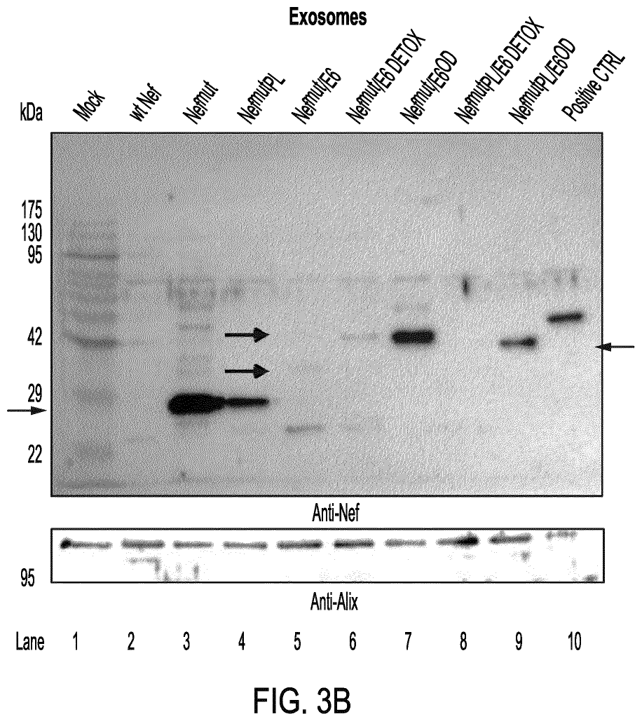

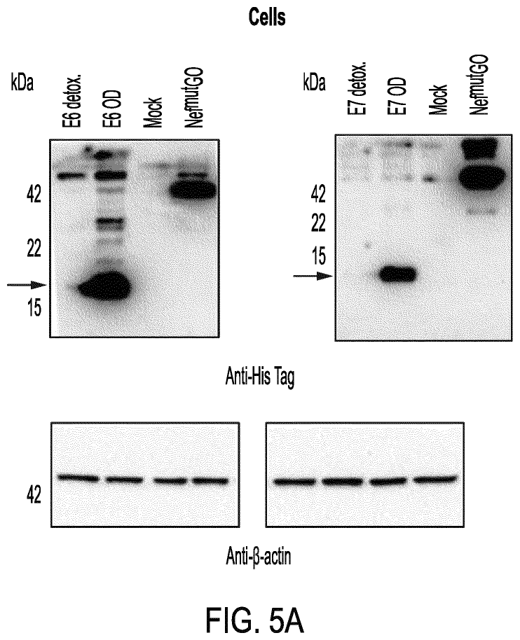

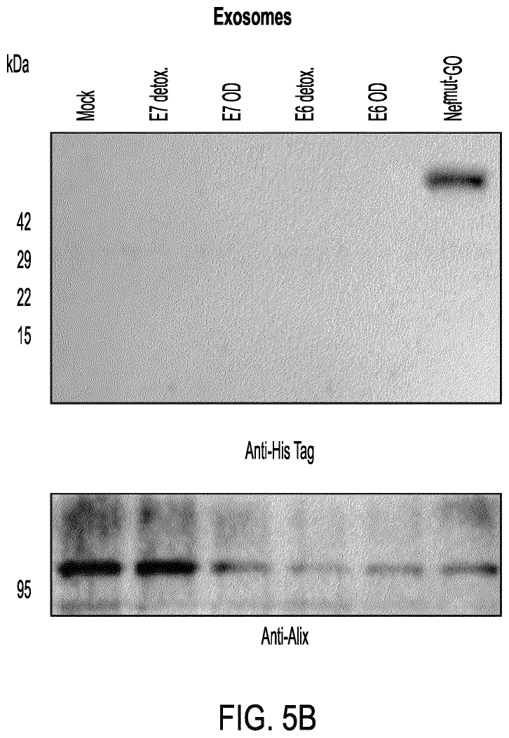

For E6 antigen, the expression in transfected cells appeared similar among all fusion products independent from codon optimization, detoxification, and truncation of Nef^{mut}. However, the efficiency of exosome uploading was different among the fusion products. In particular, Nef^{mut}/E6^{OD} and Nef^{mut}PL/E6^{OD} were most efficiently associated with exosomes (FIGS. 3A and 3B). When expressed alone, E6^{OD} but not E6^{DETOX} was detectable by western blot analysis in cells, while both were absent when exosomes were analyzed (FIGS. 5A and 5B).

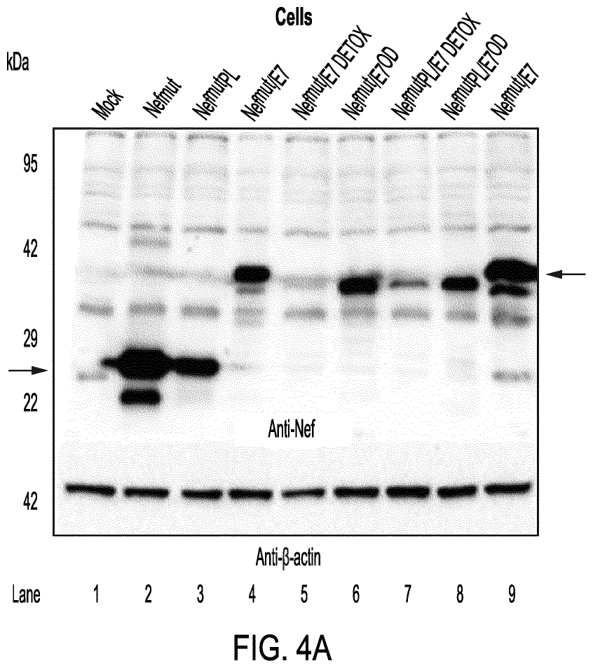

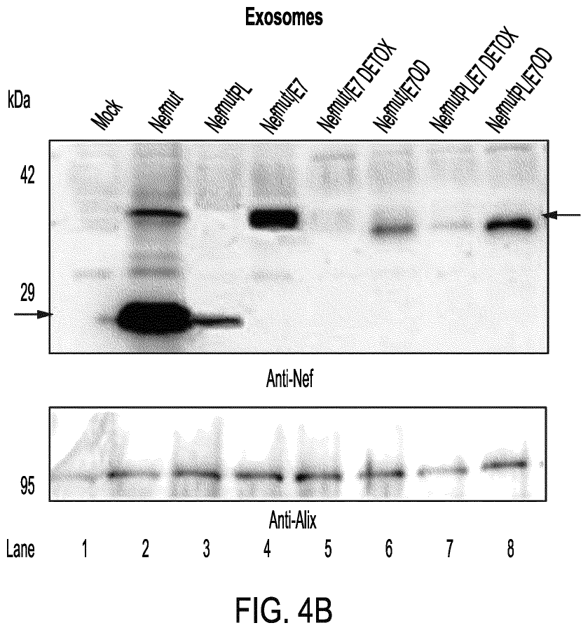

Detoxification of Nef^{mut}/E7 rendered the fusion product barely detectable in cells, and the same occurred when detoxified E7 was fused with Nef^{mut}PL. Similar to E6, both Nef^{mut}/E7^{OD} and Nef^{mut}PL/E7^{OD} were well expressed in cells and efficiently uploaded in exosomes (FIGS. 4A and 4B). When expressed alone, E7^{OD} but not E7^{DETOX} was detectable in cells by western blot analysis, while they were undetectable in exosomes (FIGS. 5A and 5B).

Both expression and exosome association of Nef^{mut}PL appeared comparable to those of full-length Nef^{mut} (FIGS. 3A and 3B; FIGS. 4A and 4B).

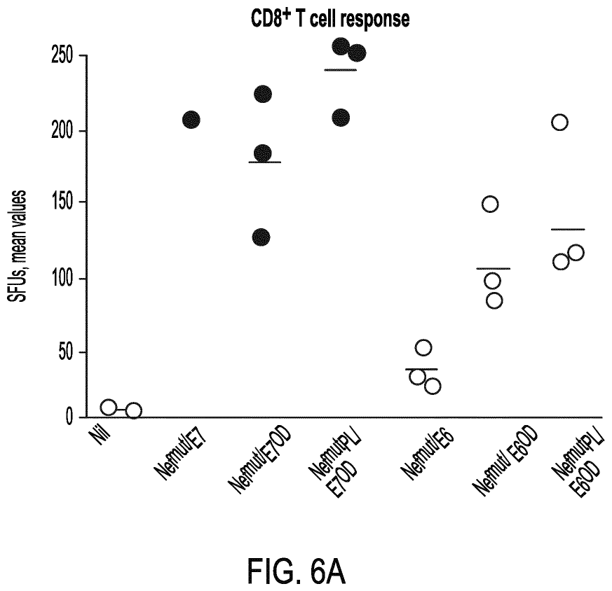

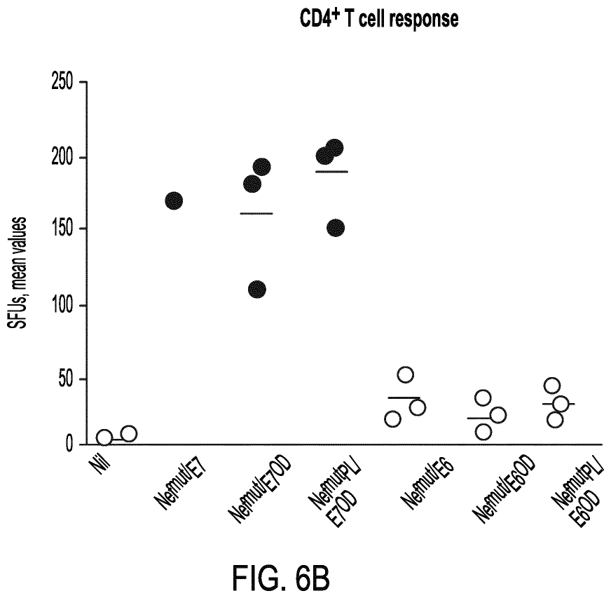

Data obtained in immunogenicity studies by IFN-γ ELISpot assay (FIGS. 6A and 6B) showed lower levels of immunogenicity of Nef^{mut}/E6 expressing vectors compared to the E7-based counterparts in general. E7 optimization and detoxification did not affect the potency of E7-specific immune response, while increased the E6-specific CD8⁺ T cell immune response. The Nef^{mut} truncation did not decrease the immunogenicity of fused products, and detected CD8⁺ T cell activation was comparable to, or higher than full-length Nef^{mut} vectors.

In order to analyze the immunization data in more detail, as well as to precisely quantitate the immune responses, ICS/flow cytometry analysis were performed on cryopreserved splenocytes. These analyses aimed at detecting peptide-activated CD8⁺ T lymphocyte regarding IFN-γ, IL-2 and TNF-α intracellular accumulation. Results were calculated as: i) percentages of CD8⁺ T cells from each injected mouse positive for each cytokine; ii) respective intragroup mean values; iii) fractions of the total CD8⁺ T cells expressing each of the possible combinations of cytokines, i.e., each single positive, triple positive, IFN-γ+IL-2, IL-2+TNF-α, and IFN-γ+TNF-α expressing cells, and iv) intragroup means thereof.

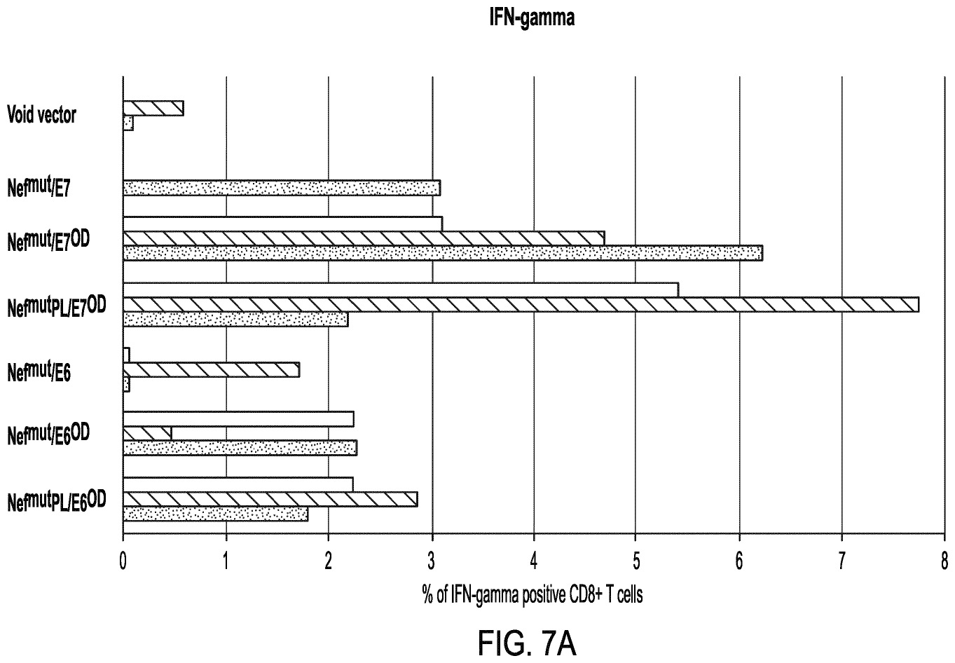

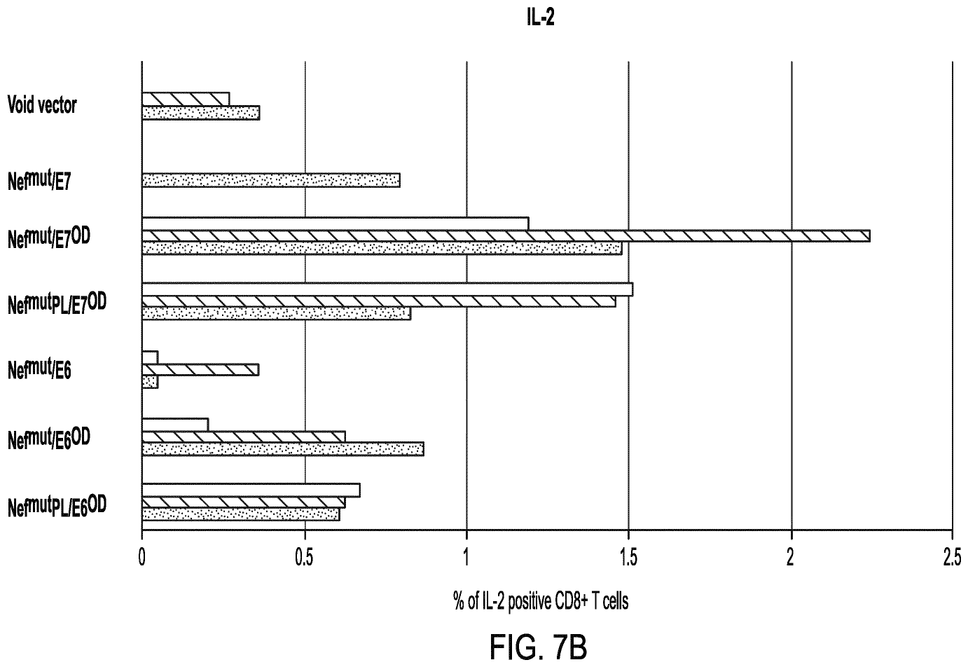

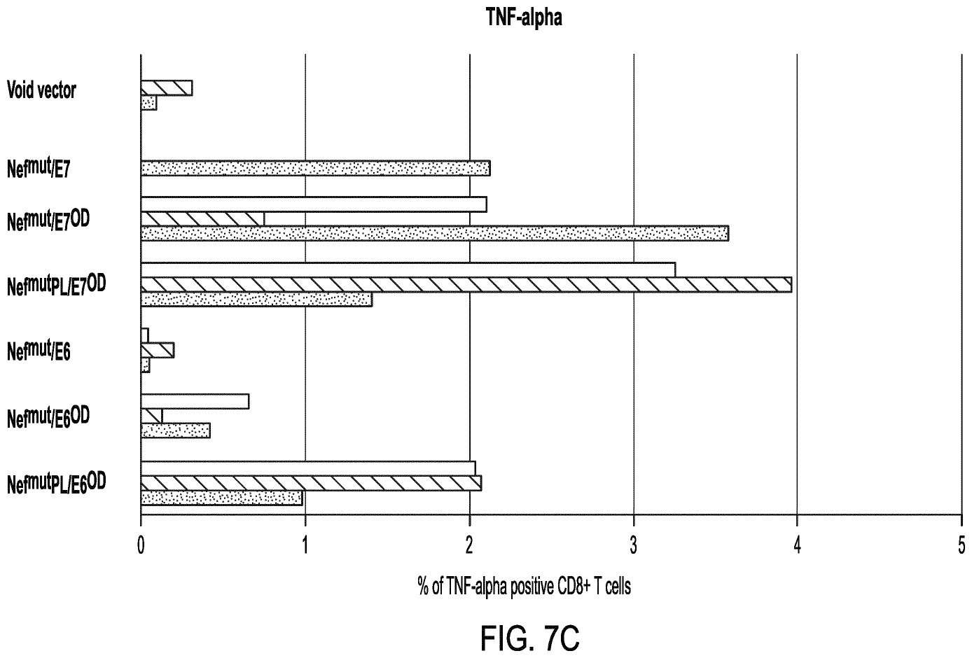

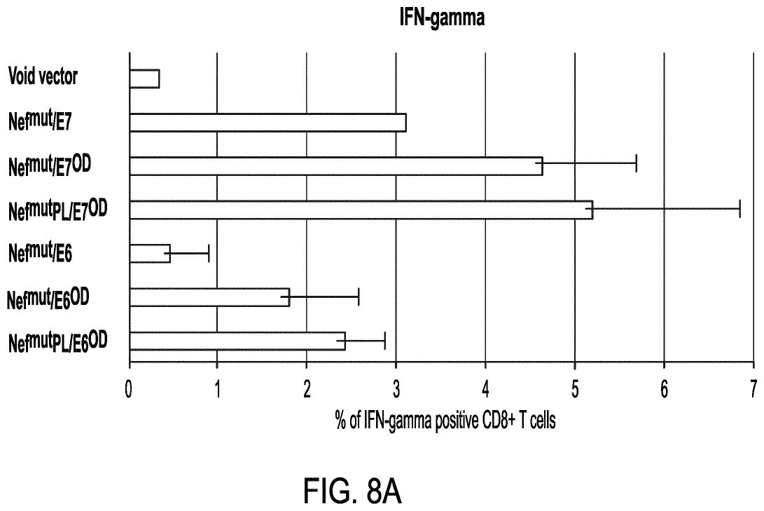

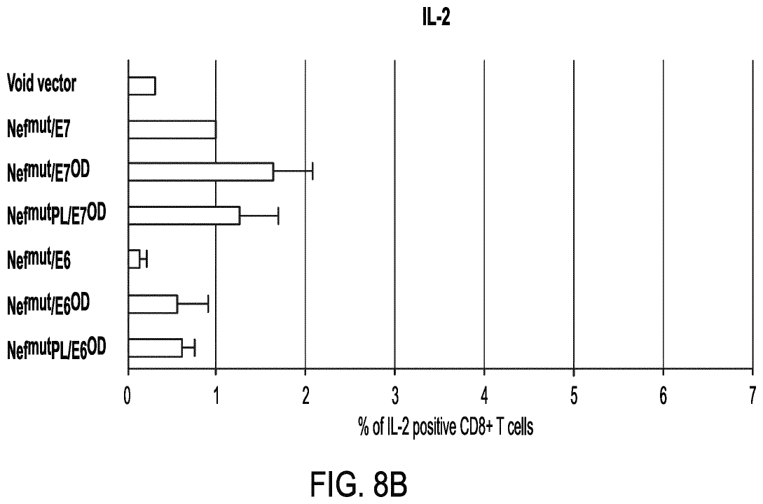

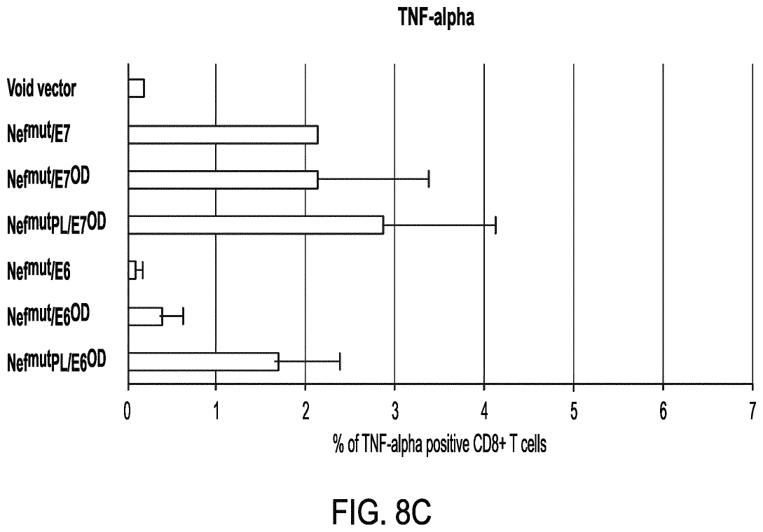

From ICS/flow cytometry analysis of the IFN-γ accumulation in CD8⁺ T cells, the percentages of IFN-γ positive CD8⁺ T cells averaged 3% of total CD8⁺ T cells in Nef^{mut}/E7 injected mice, and increased to more than 4% in the case of Nef^{mut}/E7^{OD} immunization. The Nef^{mut} truncation increased the IFN-γ induction compared to that detected in cells from mice immunized with full-length Nef^{mut}. A similar trend, however in the presence of overall lower levels of IFN-γ induction, was detected in the case of immunization with E6-derived products. Higher percentages of CD8⁺ T cells accumulating either IL-2 or TNF-α (up to 2 and 3%, respectively) were detected in CD8⁺ T cells from mice injected with E7-based DNA vectors compared to E6-based DNA vectors (0.7% for IL-2 and 1.8% for TNF-α) (FIGS. 7A-7C; FIGS. 8A-8C).

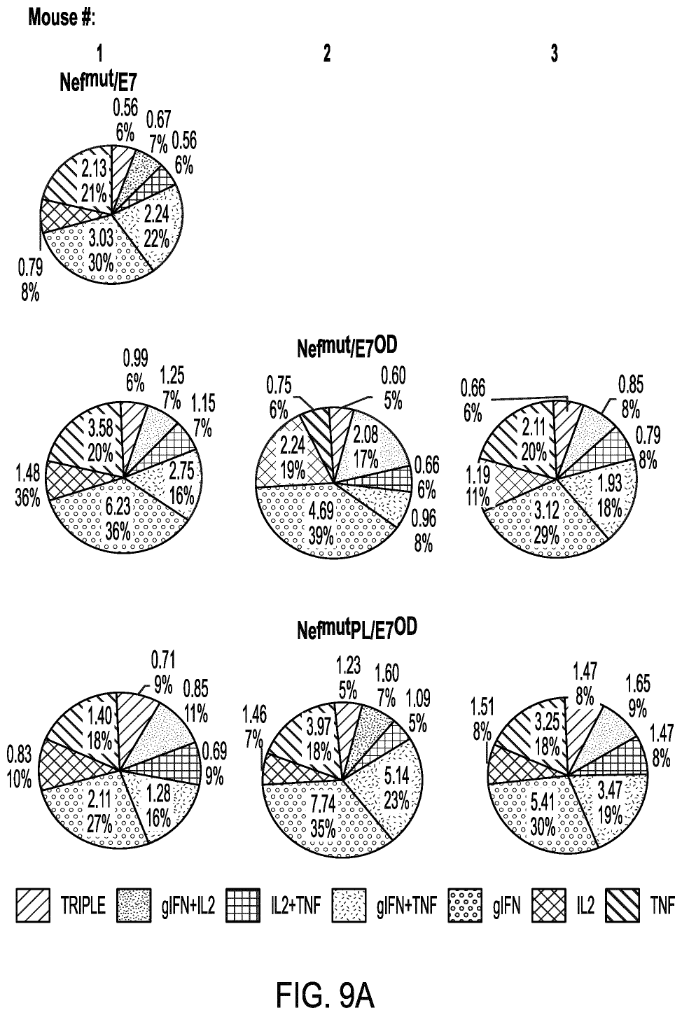

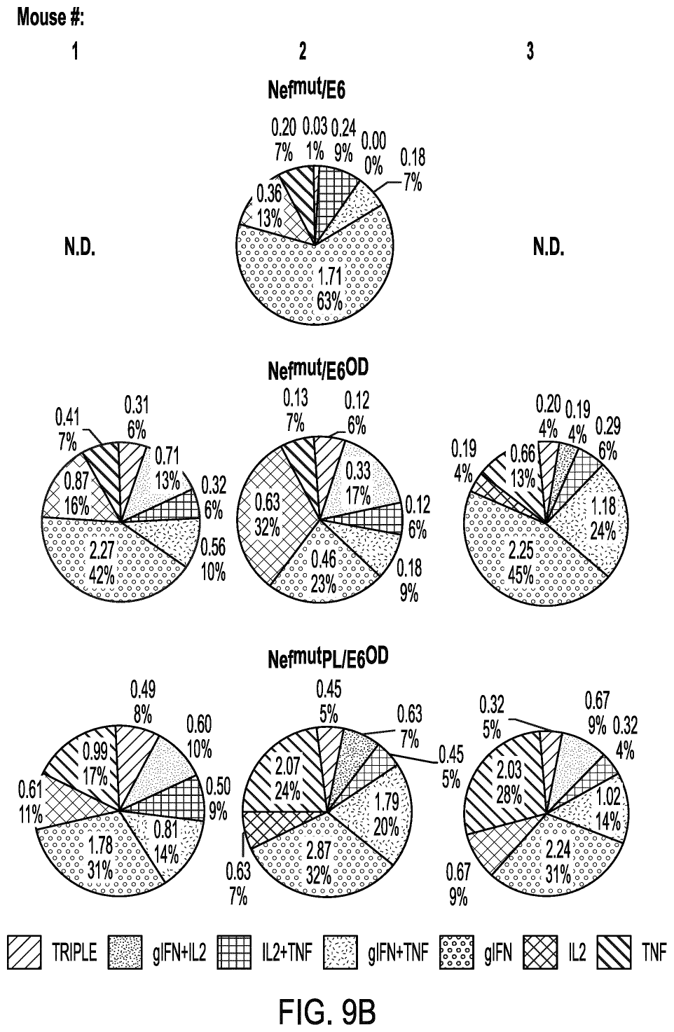

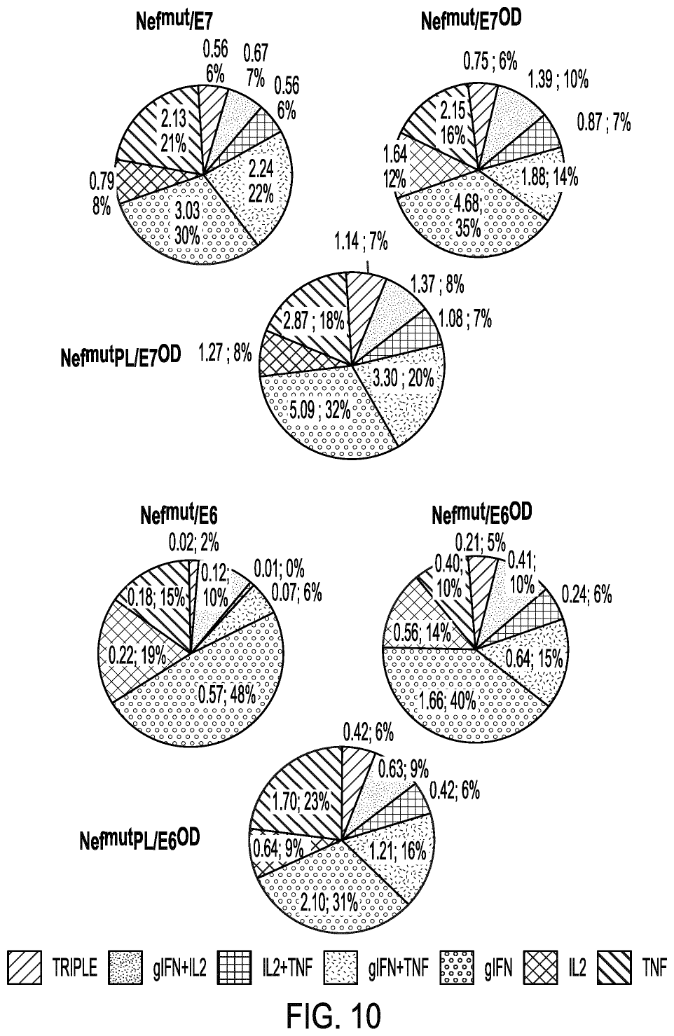

Importantly, the analysis of multiple cytokine accumulation in CD8⁺ T cells highlighted the presence of both bi- and tri-functional CD8⁺ T sub-populations in all responding mice at levels comparable to those detectable in PMA+ionomycin treated cells. In particular, triple positive CD8⁺ T cells averaged up to 7% of activated CD8⁺ T cells in the case of immunization with E7-based products, and up to 6% in the case of immunization with E6-based products. The Nef^{mut} truncation increased the percentage of triple positive CD8⁺ T cells for both E7- and E6-based fusion proteins (FIGS. 9A and 9B; FIG. 10).

The detection of both two- and three-cytokine expressing antigen-specific CD8⁺ T cells indicates that the immunizations induced a functional CD8⁺ T cell-mediated immune response potentially able to target and destroy antigen-expressing cells.

### 6.4. Example 3: Co-Immunization with Two Antigen-Expressing DNA Vectors

Co-injection of two vectors expressing distinct HPV16 antigens fused with Nef^{mut} was tested. Two DNA vectors expressing either Nef^{mut}/E6^{OD} or Nef^{mut}/E7^{OD} were injected i.m.+EP in mice either separately or in combination. The experimental design is shown in Table 4 below.

TABLE 4

| Group | Inoculum |
|---|---|
| A | i.m. + EP |
| | pcDNA3.1 empty vector |
| | 10 μg, 30 μL |
| B | i.m. + EP |
| | pTarget-Nef^{mut}/E7^{OD} |
| | 10 μg, 30 μL |
| C | i.m. + EP |
| | pTarget-Nef^{mut}/E6^{OD} |
| | 10 μg, 30 μL |
| D | i.m. + EP |
| | pTarget-Nef^{mut}/E7^{OD} + pTarget-Nef^{mut}/E6^{OD} |
| | 10 μg + 10 μg, 30 μL |

Control group A included 2 mice, groups B and C included 3 mice, and group D comprised 5 mice. Inoculations were carried out in each quadriceps and electroporation was performed after each DNA injection in all mice as described above. A second identical immunization was performed 14 days later.

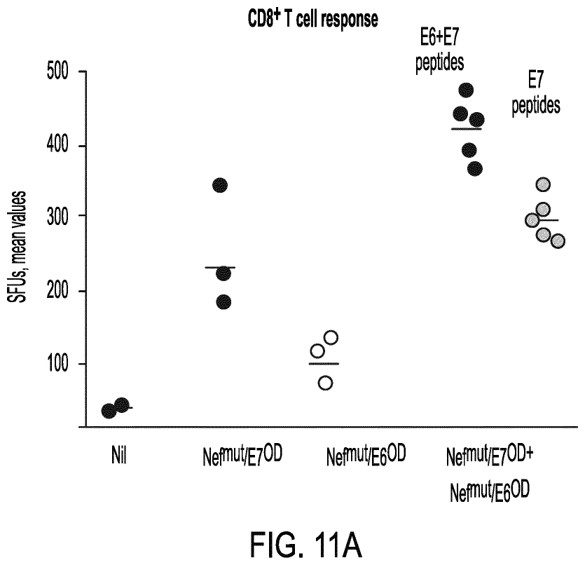

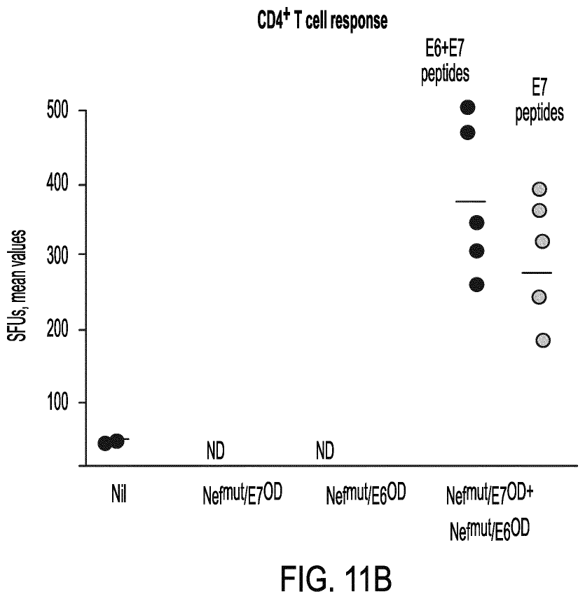

As observed in the previous experiments, results from IFN-γ ELISpot assay indicated stronger CD8⁺ T cell responses against E7 compared to E6. When the two vectors were co-injected, an additive or synergistic E6- and E7-specific immune response was generated, thus excluding possible interference effects between the injected DNA vectors. This conclusion was also supported by the evidence that the E7-specific CD8⁺ T cell response in co-injected animals was not reduced (yet resulting slightly increased) compared to that of mice injected with the Nef^{mut}/E7^{OD}-expressing vector alone (FIGS. 11A and 11B).

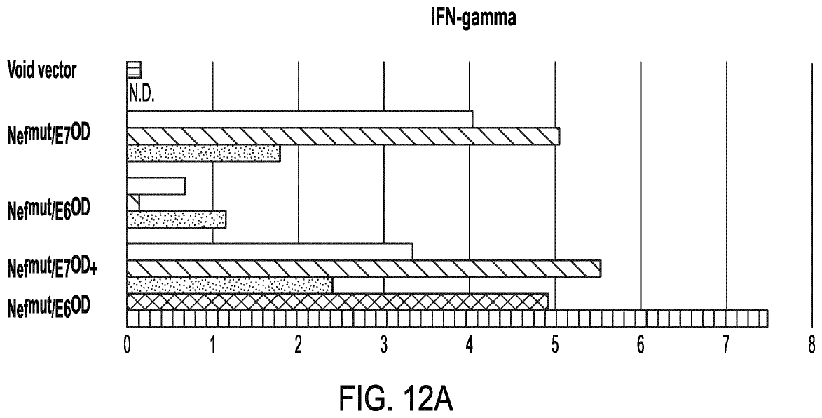

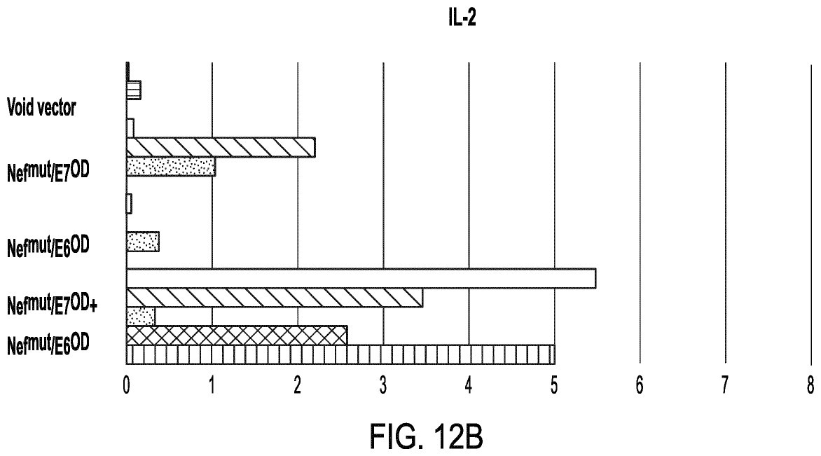

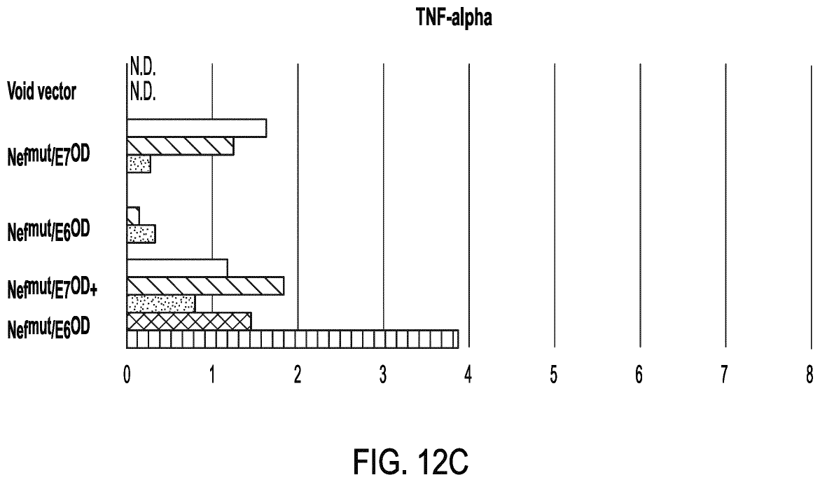

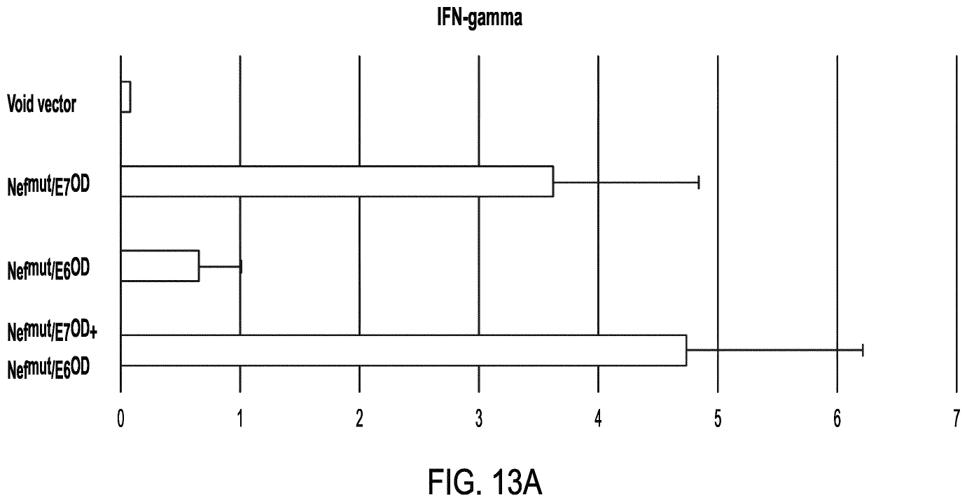

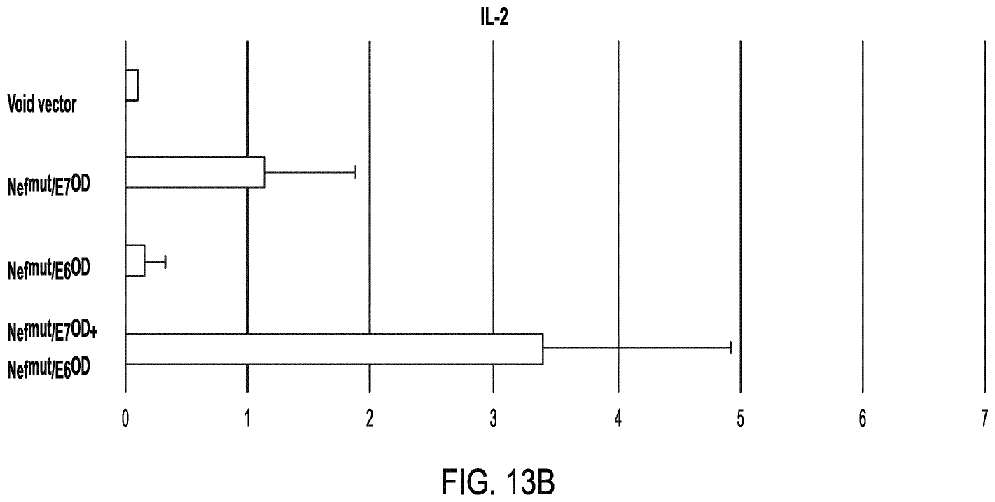

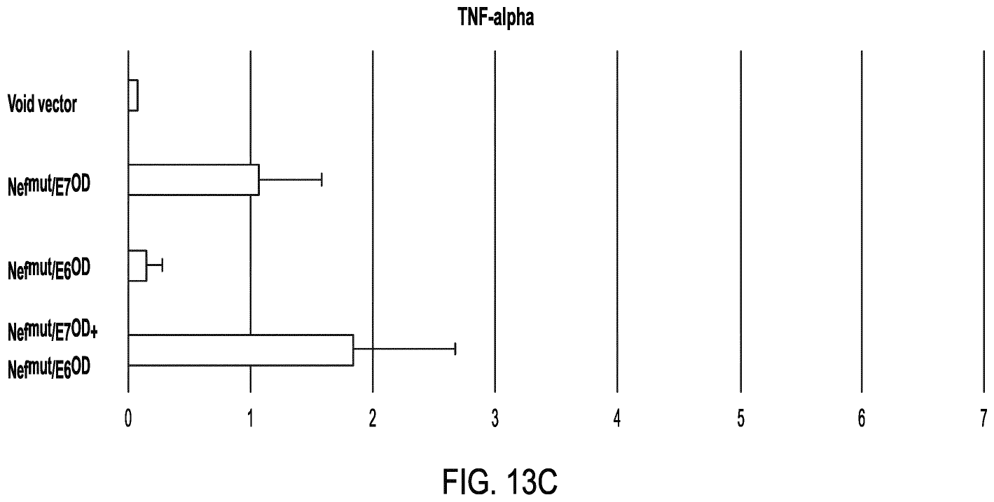

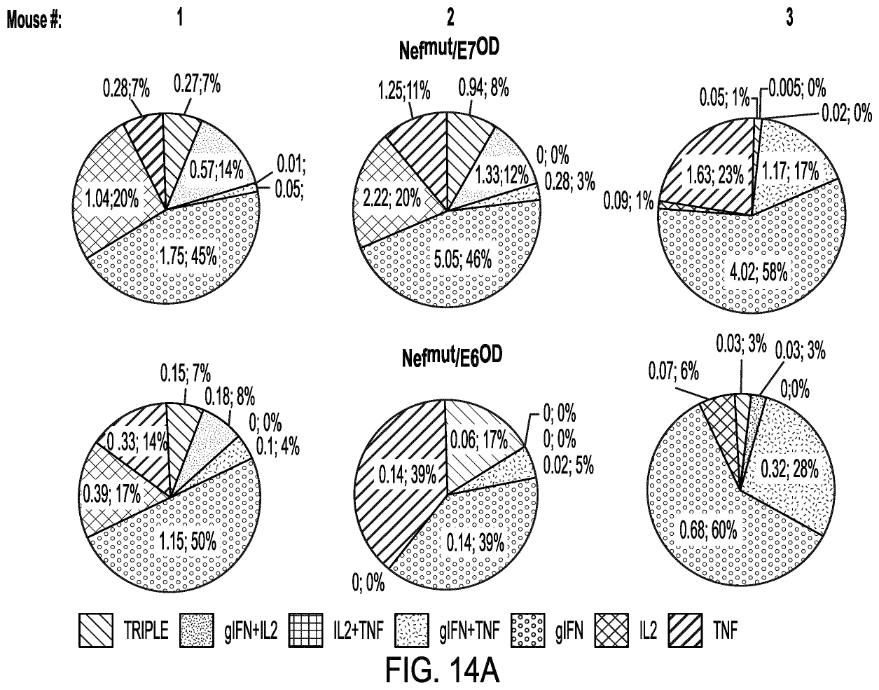

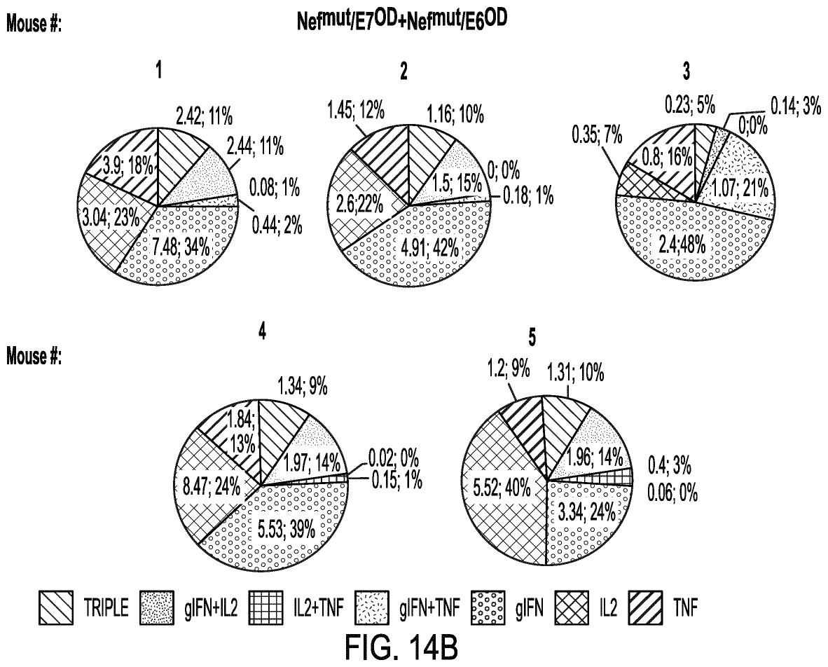

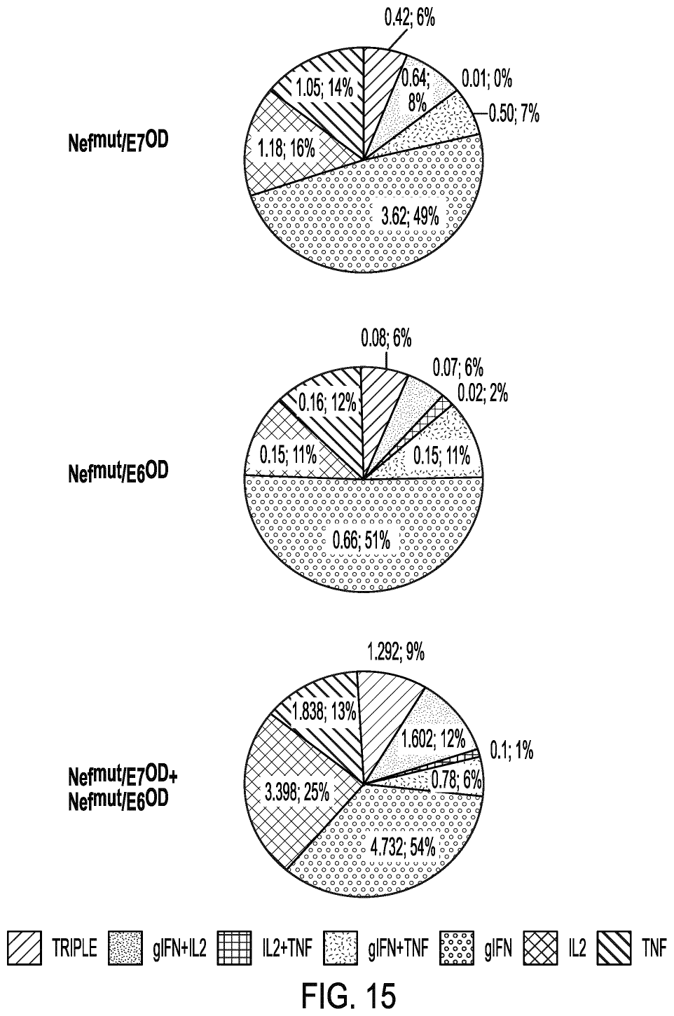

ICS/flow cytometry data on CD8⁺ T cell IFN-γ response obtained with cryopreserved splenocytes served to precisely quantitate the immune response as detected by IFN-γ ELISpot assay. The immunization with the Nef^{mut}/E7^{OD} vector resulted in 3.7% of IFN-γ producing CD8⁺ T cells, whereas the E6-specific response averaged 0.8% of CD8⁺ T cells from Nef^{mut}/E6^{OD} immunized mice. When the two vectors were co-injected, an additive or synergistic immune response reaching a mean of 4.8% of IFN-γ producing CD8⁺ T cells was induced. The E6-specific response appeared mostly towards the production of IFN-γ, whereas, upon peptide stimulation, CD8⁺ T cells from co-injected mice expressed both IL-2 (more than 3%) and TNFα (near 2%) (FIGS. 12A-12C; FIGS. 13A-13C). In addition, a mean of 9% of nonamer-activated CD8⁺ T cells from Nef^{mut}/E6^{OD} and Nef^{mut}/E7^{OD} co-injected mice co-expressed the three cytokines, strongly suggesting the induction of antigen-specific polyfunctional CTLs (FIGS. 14A and 14B; FIG. 15).

These results indicate that the immunization with two DNA vectors expressing different antigens is feasible, thus opening the possibility of immunization with multiple antigens either by using multiple vectors each expressing a different antigen, or depending on the antigen size, multiple antigens in the same vector.

### 6.5. Example 4: Benchmarking Optimized DNA Vaccine

The HPV16 E6 and E7 immunogenicity induced by injection of DNA vectors expressing either E6 or E7 ORFs alone or as fusion protein with Nef^{mut} were compared. The experimental design is shown in Table 5 below.

TABLE 5

| Group | Inoculum |
|---|---|
| A | i.m. + EP |
| | pcDNA3.1 empty vector |
| | 10 μg, 30 μL |
| B | i.m. + EP |
| | pTarget-Nef^{mut}/E7^{OD} |
| | 10 μg, 30 μL |
| C | i.m. + EP |
| | pcDNA3.1-E7^{OD} |
| | 10 μg, 30 μL |
| D | i.m. + EP |
| | pTarget-Nef^{mut}/E6^{OD} |
| | 10 μg, 30 μL |
| E | i.m. + EP |
| | pcDNA3.1-E6^{OD} |
| | 10 μg, 30 μL |

All groups included 3 mice, except control group A with 2 mice. Injections were carried out in both quadriceps and electroporation was performed after each DNA injection in all mice. A second immunization was performed 14 days later.

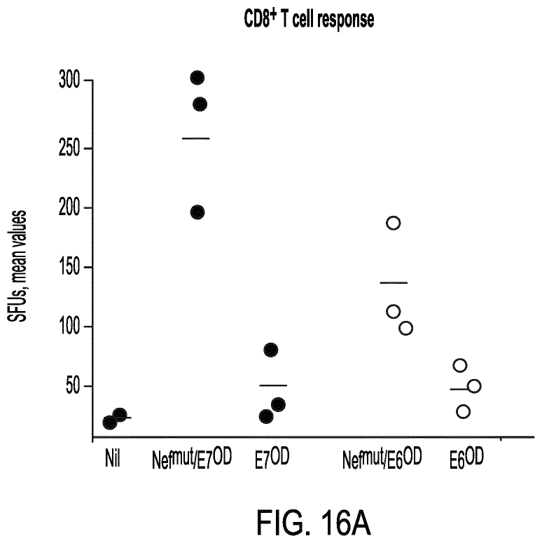

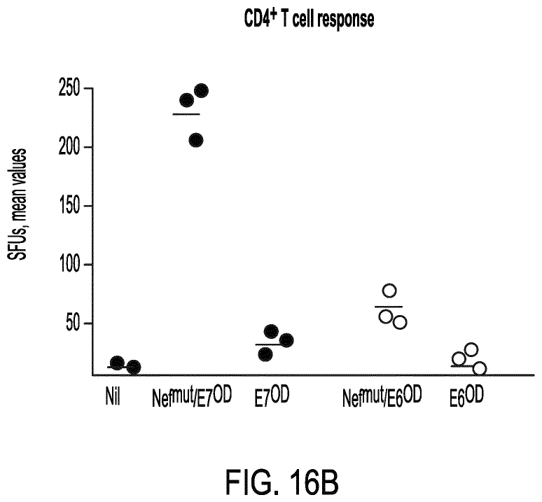

Results from IFN-γ ELISpot are shown in FIGS. 16A and 16B. The response to the vaccination with the Nef^{mut} fusion protein resulted in a ≥5-fold increase of both E7-specific CD8⁺ and CD4⁺ T cell immune responses, and about 3-fold increase of the E6-specific CD8⁺ and CD4⁺ T cell immune responses compared to the vaccination with the vectors expressing E7 and E6 without Nef^{mut}

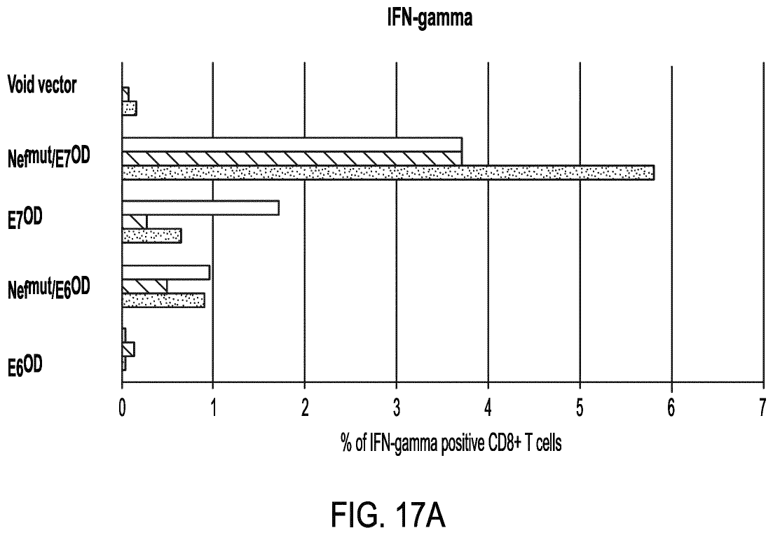

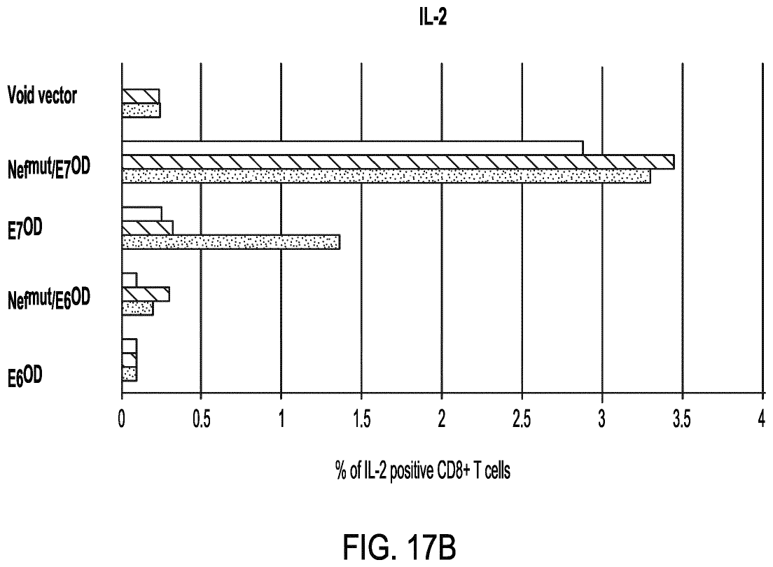

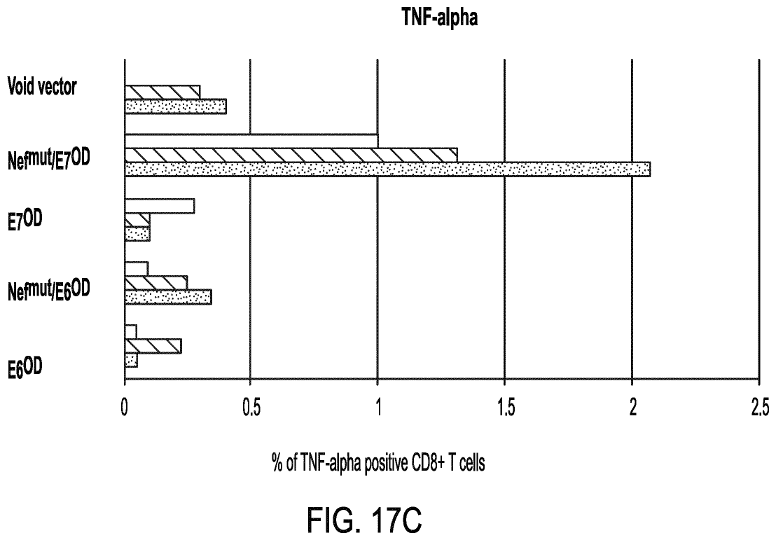

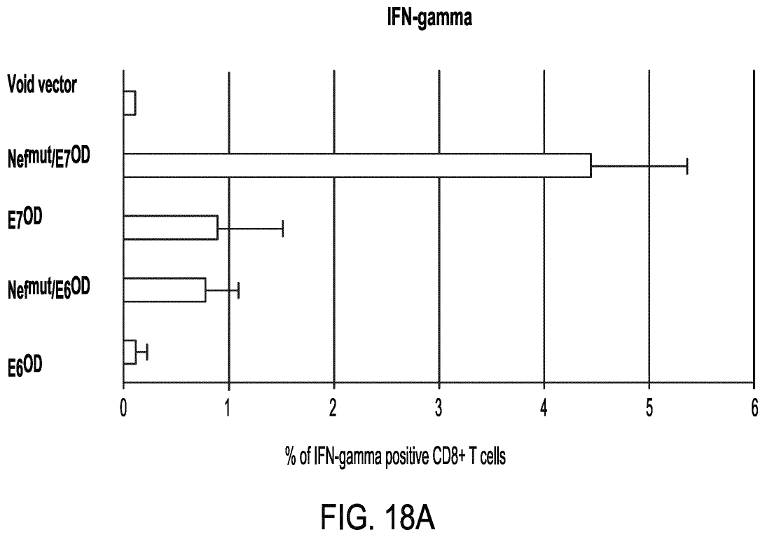

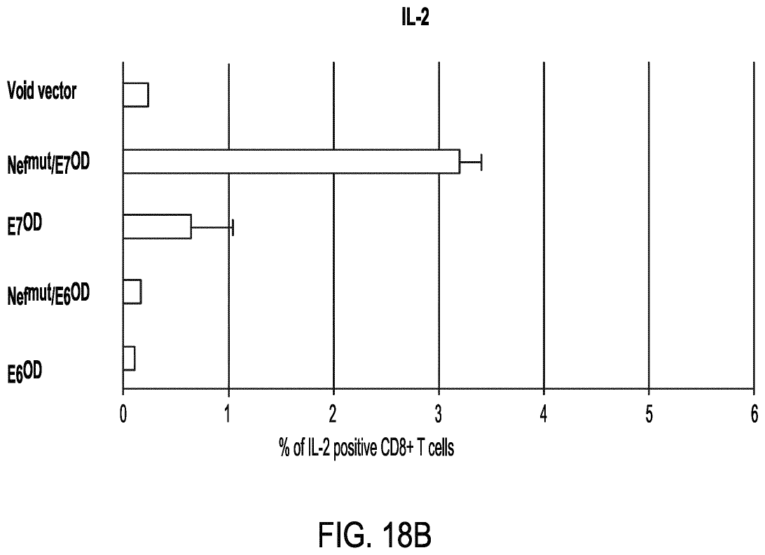

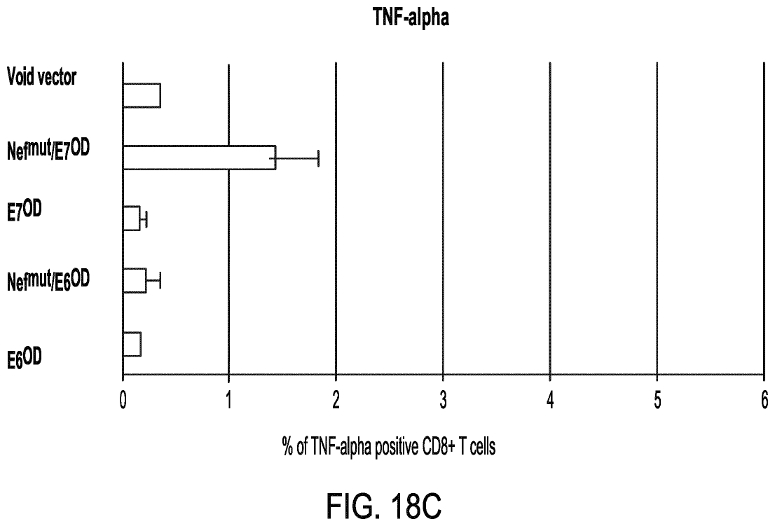

Similarly, ICS/flow cytometry assays indicated that the IFN-γ response within CD8⁺ T cells was much stronger in Nef^{mut}/E7^{OD} immunized mice, reaching a mean of more than 4% of total CD8⁺ T cells, compared to the IFN-γ response detected in splenocytes from mice immunized with E7^{OD}-expressing vector, which was below 1%. The IFN-γ response within CD8⁺ T cells from mice immunized with Nef^{mut}/E6^{OD} approached 1%, whereas it was at baseline level in mice injected with the E6^{OD}-expressing vector (FIGS. 17A-17C; FIGS. 18A-18C).

Nef^{mut}/E7^{OD} immunized mice elicited CD8⁺ T cells also expressing IL-2 (more than 5%) and TNF-α (1.4%). These percentages were significantly higher than those induced in E7^{OD} immunized mice, i.e., 0.7% and 0.2%, respectively. In all mice injected with vectors expressing E6-based products, the antigen-specific response of both IL-2 and TNF-α expression in CD8⁺ T cells appeared at the background levels, i.e. those detected in splenocytes from mice injected with the empty vector (FIGS. 17A-17C; FIGS. 18A-18C).

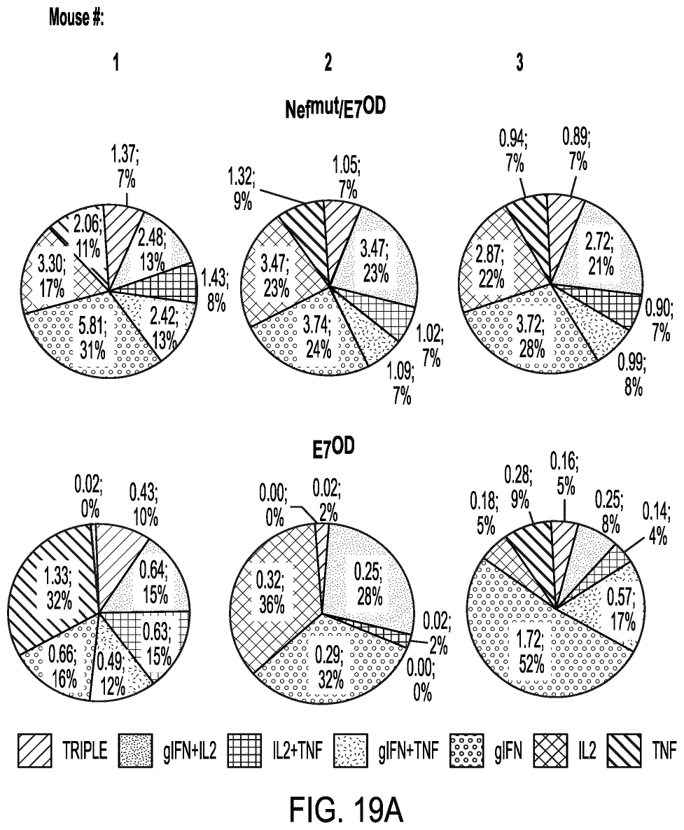

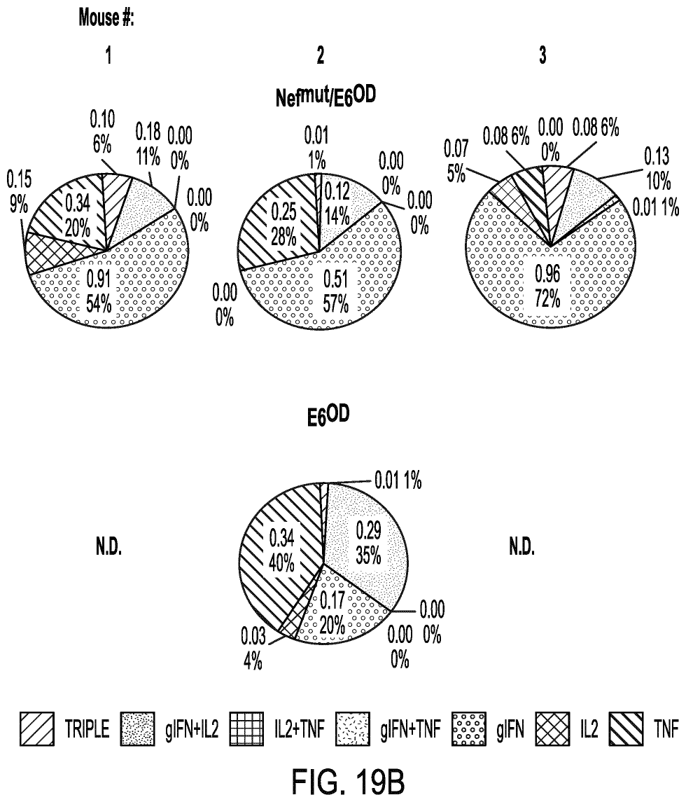

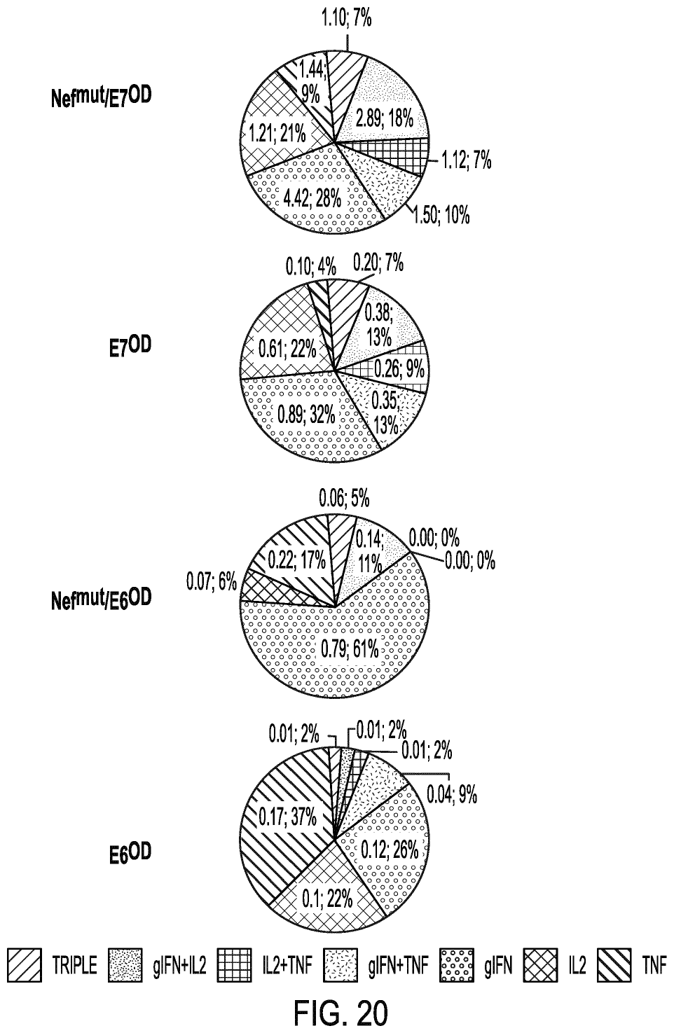

Similar percentages of tri-functional CD8⁺ T cells were detected among mice immunized with DNA vectors expressing either Nef^{mut}/E7^{OD} or E7^{OD}. The percentage of tri-functional CD8⁺ T cells increased to 5% in mice injected with Nef^{mut}/E6^{OD} compared to 1% in injected with E6^{OD} alone (FIGS. 19A and 19B; FIG. 20).

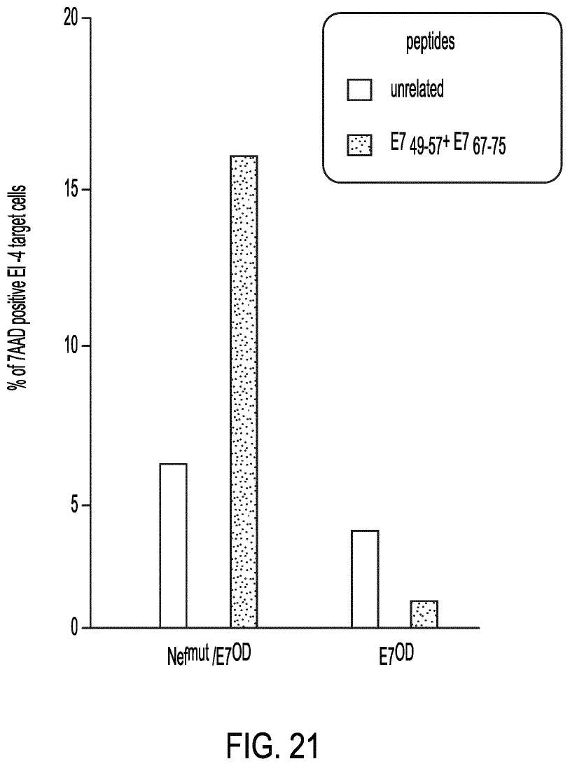

When CD8⁺ T cells isolated from splenocytes were tested for their functionality in antigen recognition by a cytotoxicity assay, CD8⁺ T cells isolated from splenocytes pooled from mice injected with Nef^{mut}/E7^{OD}-expressing vectors killed about 10% of Class I-matched cell targets. Conversely, such an effect was undetectable when CD8⁺ T cells were isolated from splenocytes pooled from mice injected with the E7^{OD}-expressing vector (FIG. 21).

### 6.6. Example 5: Anti-Tumor Therapeutic Efficacy

To assess the anti-tumor efficacy of immunization with Nef^{mut}-based vectors, the system of transplantable tumors based on syngeneic TC-1 cells implanted subcutaneously on C57BL/6 mice was used. The experimental design is shown in Table 6 below.

TABLE 6

| Group | Inoculum |
|---|---|
| A | i.m. + EP |
| | pcDNA3.1 empty vector |
| | 20 μg, 30 μL |
| B | i.m. + EP |
| | pTarget-Nef^{mut}/E6^{OD} + pTarget-Nef^{mut}/E7^{OD} |
| | 10 μg + 10 μg, 30 μL |
| C | i.m. + EP |
| | pcDNA3.1-E6^{OD} + pcDNA3.1-E7^{OD} |
| | 10 μg + 10 μg, 30 μL |
| D | i.m. + EP |
| | pTarget-Nef^{mut} |
| | 20 μg, 30 μL |

Each group included 12 mice. A total of 2×10⁵ TC-1 cells were implanted s.c. As soon as tumors became palpable, inoculations were injected in each quadriceps with EP following the DNA injection. A second immunization was performed 7 days later.

The expression of both HPV16 E6 and E7 genes in TC-1 cells used for tumor implantation was confirmed by qRT-PCR assay.

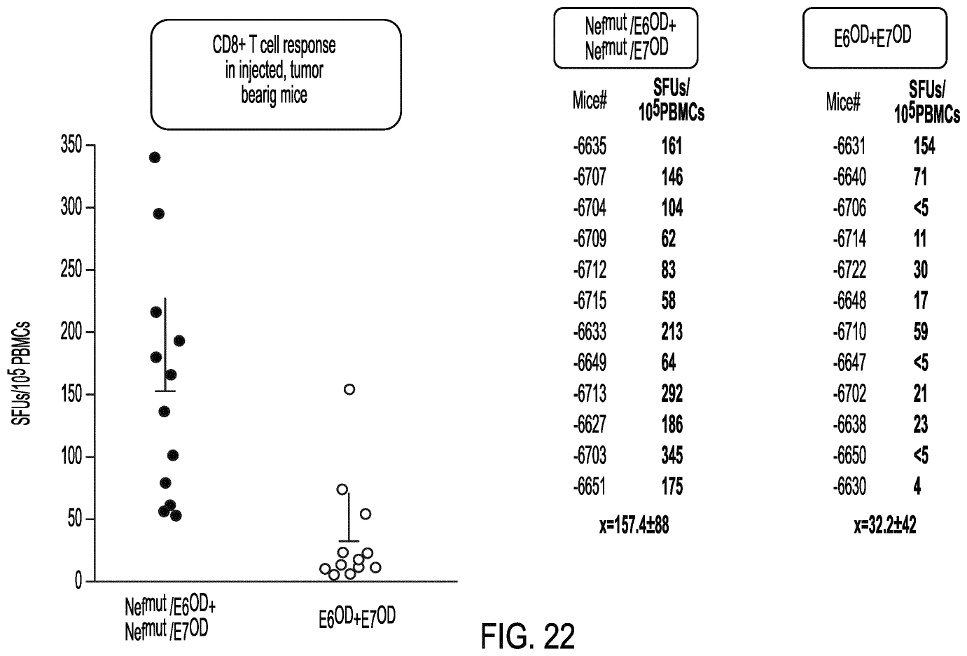

The analysis of E6- and E7-specific CD8⁺ T cell immune response by IFN-γ ELISpot assay on PBMCs recovered upon retro orbital bleedings showed an about 5-fold more potent immune response in mice injected with DNA vectors expressing the HPV16 detoxified and optimized proteins fused with Nef^{mut} compared to that detected in mice co-injected with E6^{OD} and E7^{OD}-expressing vectors (FIG. 22).

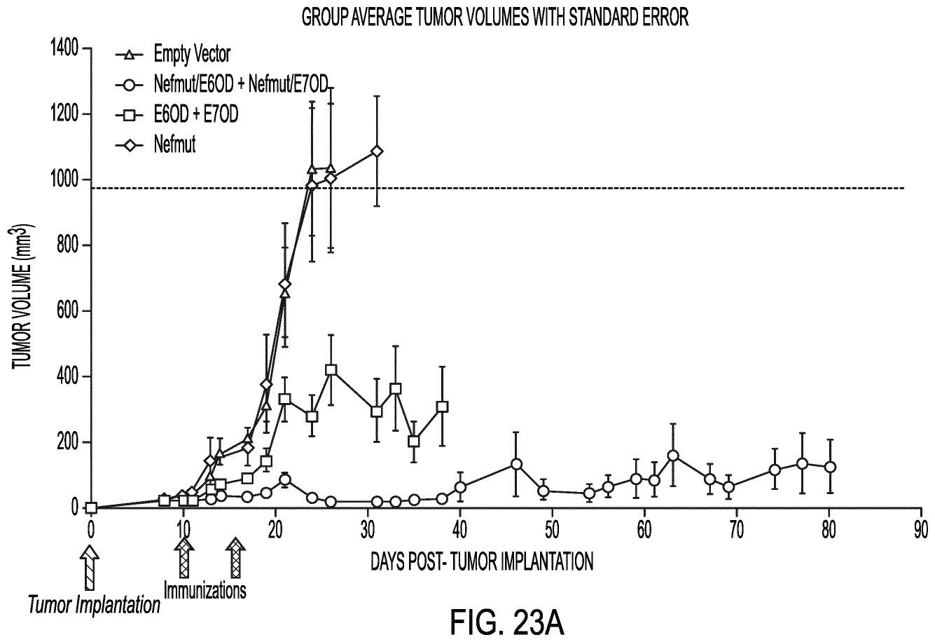

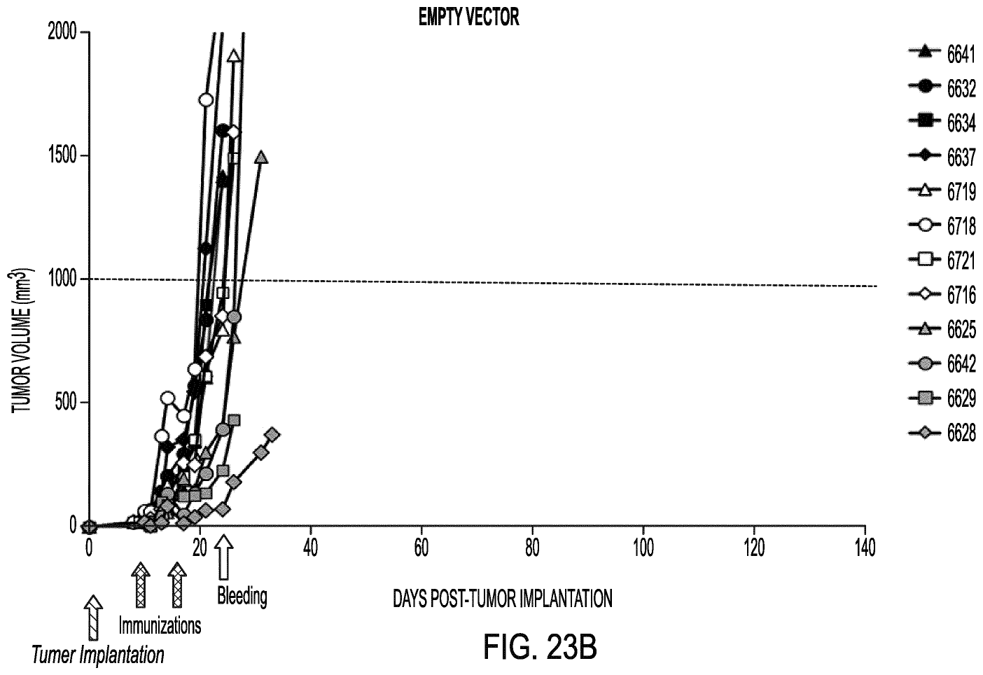

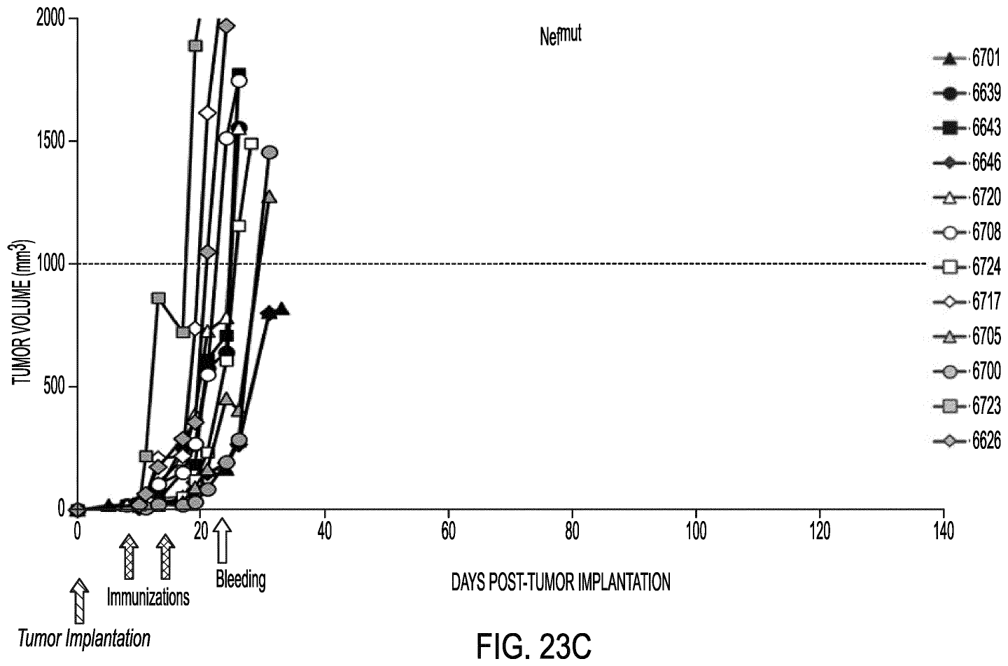

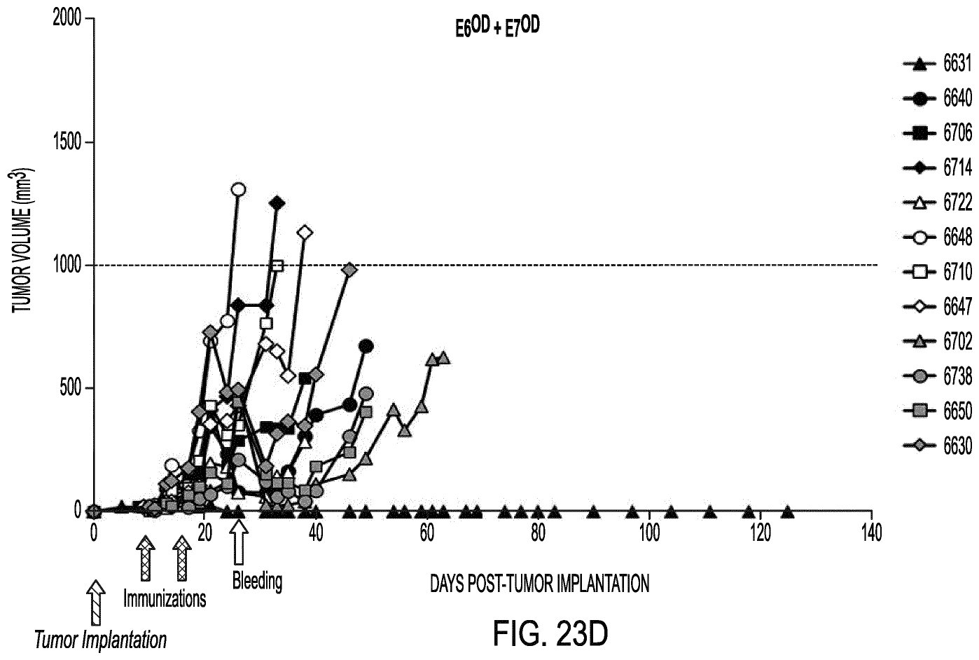

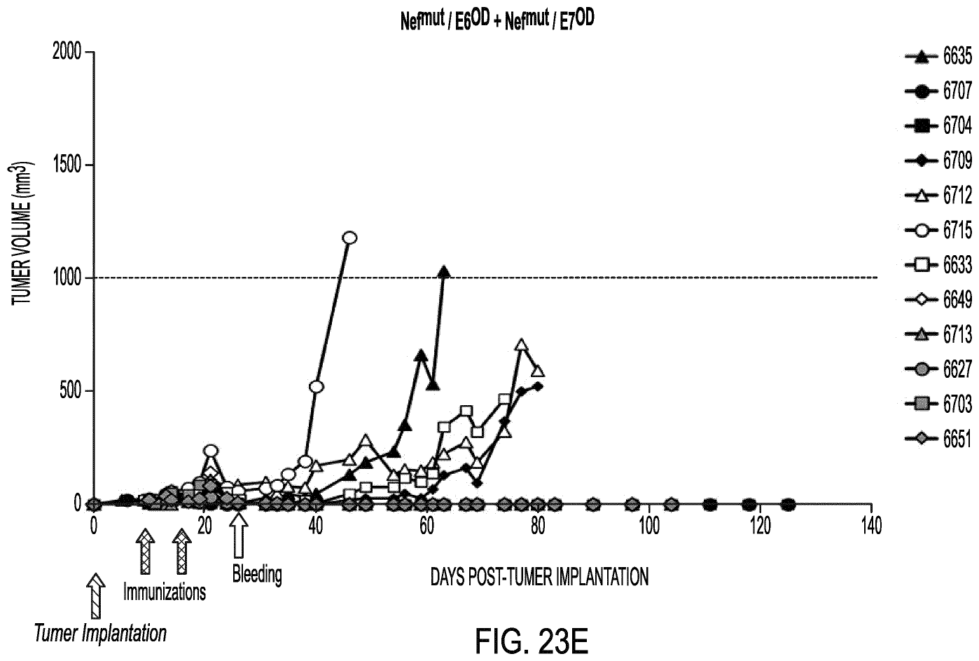

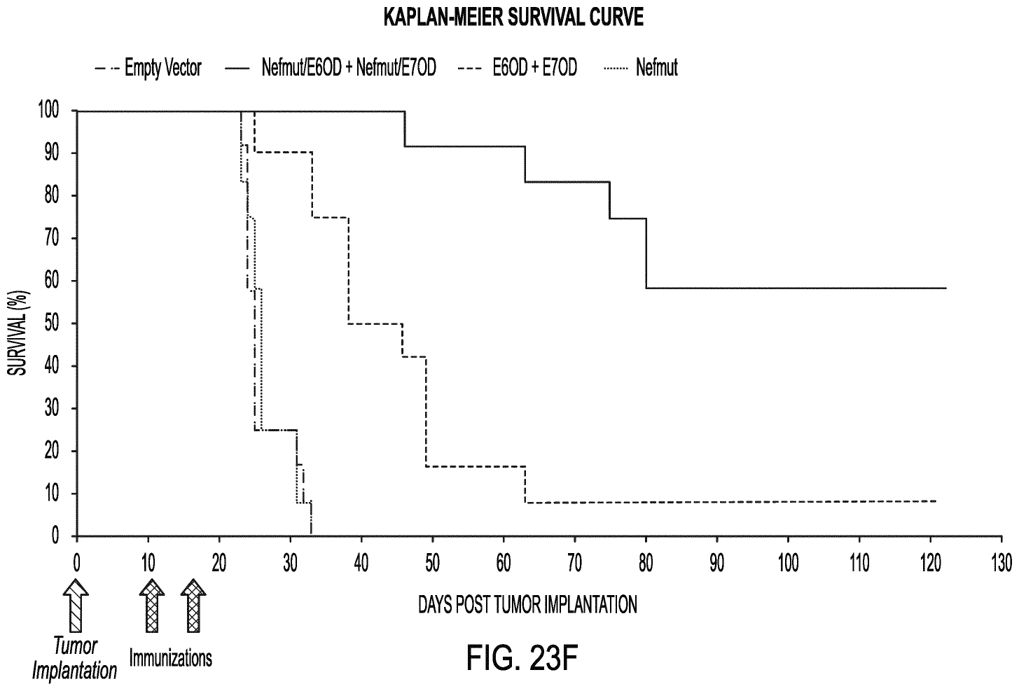

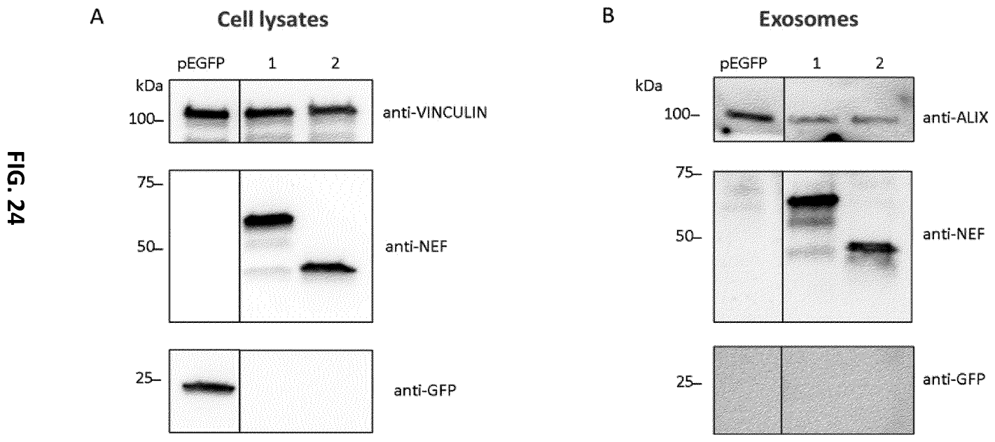

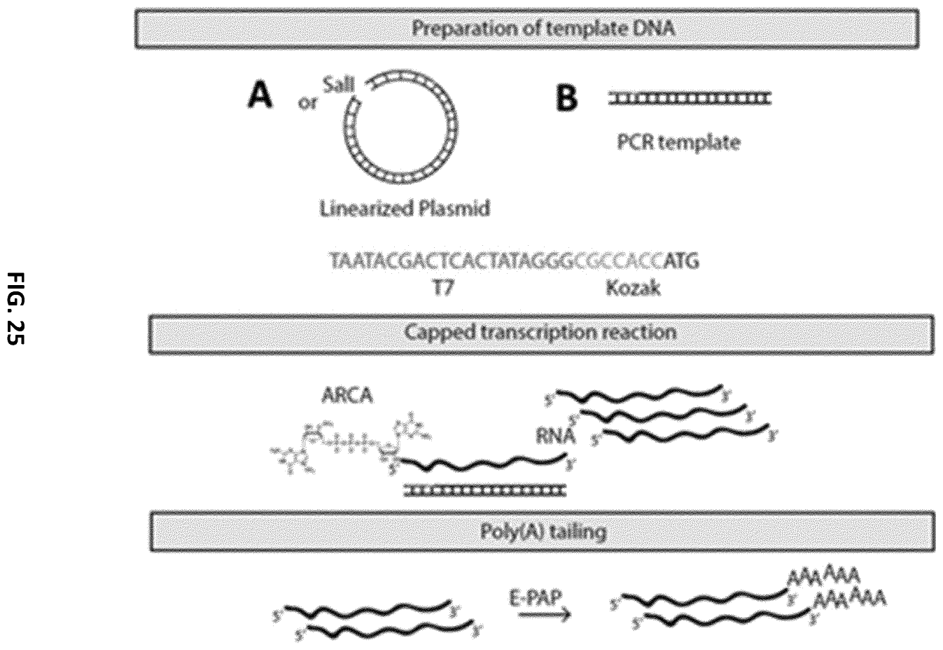

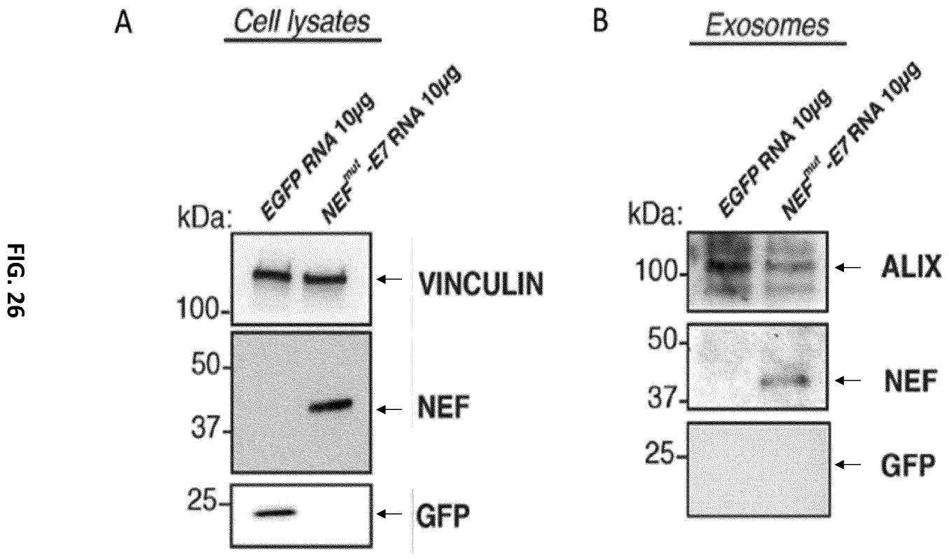

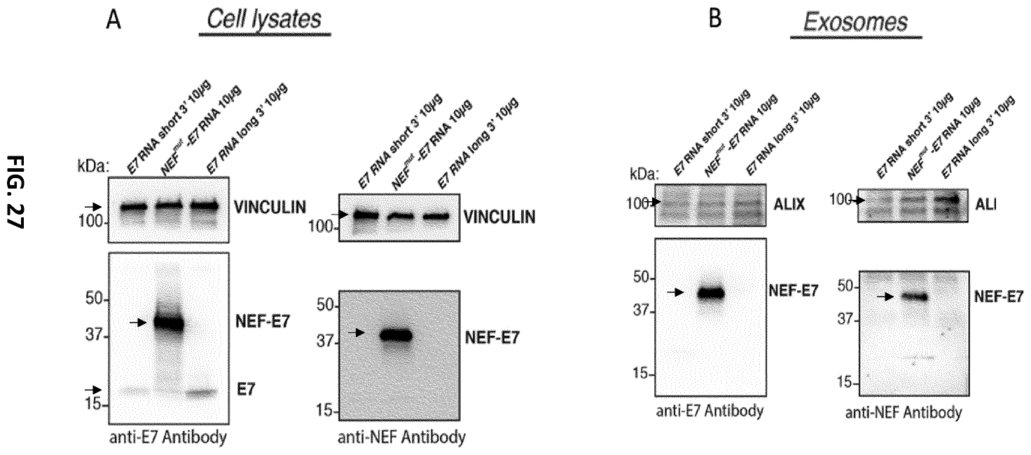

The tumor size evaluation extended until 140 days after tumor implantation indicated that the immunization with Nef^{mut}/E6^{OD} plus Nef^{mut}/E7^{OD}-expressing vectors generated an anti-tumor effect. In particular, at day 35 after tumor implantation (i.e., when all mice of control groups deceased) no or very limited tumor development was observed in Nef^{mut}/E6^{OD} plus Nef^{mut}/E7^{OD} co-injected mice. At day 65 after tumor implantation, only 1 mouse out of the 12 mice injected with DNA vectors expressing E6^{OD} and E7^{OD} was alive, whereas all mice immunized with Nef^{mut}/E6^{OD} plus Nef^{mut}/E7^{OD} still survived (FIGS. 23A-23E). As shown by the survival curve (FIG. 23F), seven mice of the group immunized with DNA vectors expressing Nef^{mut}/E7^{OD} and Nef^{mut}/E6^{OD} remained tumor-free. However, only one mouse of the group injected with DNA vectors expressing E7^{OD} and E6^{OD} Survived without developing a tumor.

FIG. 22 shows CD8⁺ T cell activation levels as detected by IFN-γ ELISpot assay on PBMCs observed on day 35 together with the efficacy data. In the group of Nef^{mut}/E7^{OD} and Nef^{mut}/E6^{OD} co-immunized mice, low levels of cell activation were detected in mice #6715, 6712, and 6709, which developed tumor. On the other hand, the highest level of CD8⁺ T cell activation in mice injected with E6^{OD} and E7^{OD}-expressing vectors was detected in the only mouse of the group (i.e., #6631) which did not develop tumor.

Taken together, these data demonstrate that the Nef^{mut}/E6^{OD}+Nef^{mut}/E7^{OD} vaccine leads to a potent tumor growth control superior than that induced by DNA vaccine expressing HPV16 E6 and E7 detoxified and optimized proteins.

### 6.7. Example 6: Nef^{mut} Fusion Vaccines Incorporating Antigens from Mycobacterium tuberculosis

6.7.1. Antigen Ag85B from Mycobacterium tuberculosis

Antigen: Ag85B
Alternative names: 30 kDa extracellular protein, 85B, Fbps B, Rv1886c,
mpt59
Organism: M. Tuberculosis/H37Rv
GenBank Accession: NP_216402
Gene ID: 885785
Length: 325
Mass (Da): 34,581

Full length Ag85B protein sequence
(SEQ ID NO: 51)
MTDVSRKIRAWGRRLMIGTAAAVVLPGLVGLAGGAATAGAFSRPGLPVEYLQVPSPSMGRDI
KVQFQSGGNNSPAVYLLDGLRAQDDYNGWDINTPAFEWYYQSGLSIVMPVGGQSSFYSDWY
SPACGKAGCQTYKWETFLTSELPQWLSANRAVKPTGSAAIGLSMAGSSAMILAAYHPQQFIY
AGSLSALLDPSQGMGPSLIGLAMGDAGGYKAADMWGPSSDPAWERNDPTQQIPKLVANNTR
LWVYCGNGTPNELGGANIPAEFLENFVRSSNLKFQDAYNAAGGHNAVFNFPPNGTHSWEYW
GAQLNAMKGDLQS SLGAG

Domain Features:

Signal peptide (residues 1-40) (SEQ ID NO: 52):
MTDVSRKIRAWGRRLMIGTAAAVVLPGLVGLAGGAATAGA
Chain (residues 41-325) (SEQ ID NO: 53):
FSRPGLPVEYLQVPSPSMGRDIKVQFQSGGNNSPAVYLLDGLRAQDDYNGWDINTPAFEWYY
QSGLSIVMPVGGQSSFYSDWYSPACGKAGCQTYKWETFLTSELPQWLSANRAVKPTGSAAIG
LSMAGSSAMILAAYHPQQFIYAGSLSALLDPSQGMGPSLIGLAMGDAGGYKAADMWGPSSDP
AWERNDPTQQIPKLVANNTRLWVYCGNGTPNELGGANIPAEFLENFVRSSNLKFQDAYNAAG
GHNAVFNFPPNGTHSWEYWGAQLNAMKGDLQSSLGAG

To design a fusion protein comprising, from N-terminus to C-terminus, an exosome-anchoring domain and immunogenic antigen domain, where the immunogenic antigen domain is M. Tuberculosis antigen 85B, we:
removed the N-terminal signal peptide (residues 1-40)
optimized the nucleic acid encoding sequence according to mammalian codon usage to improve expression: synthetic Ag85B DNA (minus signal peptide) using the codons that appear most frequently in humans, without altering amino acid sequences of Ag85A-encoded protein (GenSmart Optimization, GenScript)
cloned codon-optimized polynucleotide encoding Ag85B (minus signal peptide) into pVAX-1-Nefmut EcoRI/ApaI sites (pVAX-1 from GenScript)
added Kozak consensus sequence,
   to produce a plasmid vector encoding fusion protein Nef^{mut}+Ag85B (495 aa) (SEQ ID NO: 54):

MGCKWSKSSVVGWPAVRERMRRAEPAADGVGAASRDLEKHGAITSSNTAATNADCAWLEAQEEEEVGFP
VTPQVPLRPMTYKAAVDLSHFLKEKGGLEGLIHSQRRQDILDLWIYHTQGYFPDWQNYTPGPGIRYPLT
FGWCYKLVPVEPEKLEEANKGENTSLLHPVSLHGMDDPGREVLEWRFDSRLAFHHVARELHPEYFKNCG
PGPFSRPGLPVEYLQVPSPSMGRDIKVQFQSGGNNSPAVYLLDGLRAQDDYNGWDINTPAFEWYYQSGL
SIVMPVGGQSSFYSDWYSPACGKAGCQTYKWETFLTSELPQWLSANRAVKPTGSAAIGLSMAGSSAMIL
AAYHPQQFIYAGSLSALLDPSQGMGPSLIGLAMGDAGGYKAADMWGPSSDPAWERNDPTQQIPKLVANN
TRLWVYCGNGTPNELGGANIPAEFLENFVRSSNLKFQDAYNAAGGHNAVFNFPPNGTHSWEYWGAQLNA
MKGDLQSSLGAG

where the N-terminal Nef^{mut} domain is underlined and in bold, the 4 amino acid linker GPGP is double underlined, and the C-terminal Ag85B antigen (without signal sequence) is in italics.

The encoding Nef^{mut}+Ag85B DNA sequence is (SEQ ID NO: 74):

ATGGGTTGCAAGTGGTCAAAAAGTAGTGTGGTTGGATGGCCTGCTGTAAGGGAAAGAATGAGACGAGCT
GAGCCAGCAGCAGATGGGGTGGGAGCAGCATCTCGAGACCTAGAAAAACATGGAGCAATCACAAGTAGC
AATACAGCAGCTACCAATGCTGATTGTGCCTGGCTAGAAGCACAAGAGGAGGAGGAGGTGGGTTTTCCA
GTCACACCTCAGGTACCTTTAAGACCAATGACTTACAAGGCAGCTGTAGATCTTAGCCACTTTTTAAAA
GAAAAGGGGGGACTGGAAGGGCTAATTCACTCCCAACGAAGACAAGATATCCTTGATCTGTGGATCTAC
CACACACAAGGCTACTTCCCTGATTGGCAGAACTACACACCAGGACCAGGGATCAGATATCCACTGACC
TTTGGATGGTGCTACAAGCTAGTACCAGTTGAGCCAGAGAAGTTAGAAGAAGCCAACAAAGGAGAGAAC
ACCAGCTTGTTACACCCTGTGAGCCTGCATGGAATGGATGACCCGGGGAGAGAAGTGTTAGAGTGGAGG
TTTGACAGCCGCCTAGCATTTCATCACGTGGCCCGAGAGCTGCATCCGGAGTACTTCAAGAACTGCGGA
CCTGGGCCCTTTAGCAGGCCTGGCCTGCCCGTGGAATACCTGCAAGTGCCTTCCCCTAGCATGGGAAGA
GATATTAAGGTGCAGTTCCAGAGCGGCGGAAACAACAGCCCCGCTGTTTACCTGCTCGACGGCCTGAGA
GCCCAGGATGACTACAACGGCTGGGACATCAACACCCCTGCCTTCGAGTGGTACTACCAGTCTGGCCTG
TCTATCGTGATGCCAGTGGGCGGCCAGAGCAGCTTCTACAGCGACTGGTATAGCCCTGCCTGCGGCAAG
GCCGGTTGTCAGACCTACAAATGGGAGACATTCCTGACCAGCGAGCTGCCTCAGTGGCTGTCCGCCAAT
CGGGCGGTCAAGCCAACCGGCAGCGCCGCTATCGGCCTGAGCATGGCCGGCAGCAGCGCCATGATCCTG
GCCGCTTATCACCCCCAACAATTTATCTACGCCGGCTCCCTGAGCGCCCTGCTGGACCCCAGCCAGGGC
ATGGGACCTAGCCTGATCGGACTTGCTATGGGCGATGCTGGAGGCTACAAGGCCGCCGACATGTGGGGA
CCTTCTTCTGATCCTGCCTGGGAGAGAAACGACCCTACACAGCAGATCCCCAAGCTGGTGGCCAACAAT
ACCAGACTGTGGGTGTACTGCGGCAACGGAACACCTAACGAGCTGGGCGGAGCCAACATCCCTGCCGAG
TTCCTGGAAAACTTCGTGCGGAGCTCTAATCTGAAGTTCCAGGACGCCTACAATGCCGCCGGCGGCCAC
AACGCCGTGTTCAACTTCCCACCTAACGGCACCCACAGCTGGGAATACTGGGGCGCTCAGCTGAACGCC
ATGAAAGGCGACCTGCAGTCCTCTCTGGGAGCCGGATGA

6.7.2. Antigen ESAT-6 from Mycobacterium tuberculosis

Antigen: ESAT-6
Alternative names: esxA, Rv3875
Organism: M. Tuberculosis/H37Rv
NCBI Reference Sequence (protein): YP_178023.1

NCBI Reference Sequence (DNA): NC_000962.3

Gene ID: 886209

Length: 95

Mass (Da): 9,904

Full length ESAT-6 protein sequence
(SEQ ID NO: 75)
MTEQQWNFAGIEAAASAIQGNVTSIHSLLDEGKQSLTKLAAAWGGSGSE
AYQGVQQKWDATATELNNALQNLARTISEAGQAMASTEGNVTGMFA

Domain Features:

Transmembrane domain (aa 11-43)
(SEQ ID NO: 76)
IEAAASAIQGNVTSIHSLLDEGKQSLTKLAAAW
Transmembrane (aa 49-85) (SEQ ID NO: 77):
EAYQGVOQKWDATATELNNALQNLARTISEAGQAMAS

To design a fusion protein comprising, from N-terminus to C-terminus, an exosome-anchoring domain and immunogenic antigen domain, where the immunogenic antigen domain is M. Tuberculosis ESAT-6, we:
used full length sequence, as removal of the transmembrane (TM) domains (to avoid protein insertion into the membranes) would remove a very large portion of the epitopes and the remaining protein might not be adequately immunogenic.
optimized the coding sequence according to mammalian codon usage to improve the expression: synthetic ESAT-6 DNA using the codons which most frequently appear in humans, without altering amino acid sequences of ESAT-6 encoded protein (GenSmart Optimization, GenScript).
cloned codon-optimized polynucleotide encoding ESAT-6 in pVAX-1-Nefmut (pVAX-1 from GenScript)
added Kozak consensus sequence,
   to produce a plasmid vector encoding fusion protein Nef^{mut}+ESAT-6 fusion protein (304 aa) (SEQ ID NO: 78):

MGCKWSKSSVVGWPAVRERMRRAEPAADGVGAASRDLEKHGAITSSNTAATNADCAWLEAQEEEEVGE
PVTPQVPLRPMTYKAAVDLSHFLKEKGGLEGLIHSQRRQDILDLWIYHTQGYFPDWQNYTPGPGIRYP
LTFGWCYKLVPVEPEKLEEANKGENTSLLHPVSLHGMDDPGREVLEWRFDSRLAFHHVARELHPEYFK
NCGPGPTEQQWNFAGIEAAASAIQGNVTSIHSLLDEGKQSLTKLAAAWGGSGSEAYQGVQQKWDATAT
ELNNALQNLARTISEAGQAMASTEGNVTGMEA

The encoding Nef^{mut}+ESAT-6 DNA sequence is (SEQ ID NO: 79):

ATGGGTTGCAAGTGGTCAAAAAGTAGTGTGGTTGGATGGCCTGCTGTAAGGGAAAGAATGAGACGAGCT
GAGCCAGCAGCAGATGGGGTGGGAGCAGCATCTCGAGACCTAGAAAAACATGGAGCAATCACAAGTAGC
AATACAGCAGCTACCAATGCTGATTGTGCCTGGCTAGAAGCACAAGAGGAGGAGGAGGTGGGTTTTCCA
GTCACACCTCAGGTACCTTTAAGACCAATGACTTACAAGGCAGCTGTAGATCTTAGCCACTTTTTAAAA
GAAAAGGGGGGACTGGAAGGGCTAATTCACTCCCAACGAAGACAAGATATCCTTGATCTGTGGATCTAC
CACACACAAGGCTACTTCCCTGATTGGCAGAACTACACACCAGGACCAGGGATCAGATATCCACTGACC
TTTGGATGGTGCTACAAGCTAGTACCAGTTGAGCCAGAGAAGTTAGAAGAAGCCAACAAAGGAGAGAAC
ACCAGCTTGTTACACCCTGTGAGCCTGCATGGAATGGATGACCCGGGGAGAGAAGTGTTAGAGTGGAGG
TTTGACAGCCGCCTAGCATTTCATCACGTGGCCCGAGAGCTGCATCCGGAGTACTTCAAGAACTGCGGA
CCTGGGCCCACCGAGCAGCAATGGAACTTCGCCGGCATCGAGGCCGCTGCTTCCGCCATCCAGGGCAAC
GTGACCAGCATCCACAGCCTGCTGGACGAGGGCAAGCAGAGCCTGACCAAGCTGGCCGCCGCTTGGGGC
GGATCTGGCTCTGAAGCCTACCAGGGCGTGCAGCAGAAATGGGATGCCACAGCCACAGAACTGAACAAC
GCCCTGCAGAACCTGGCTAGAACCATCAGCGAGGCCGGACAGGCCATGGCCAGCACCGAAGGCAATGTG
ACAGGCATGTTCGCCTGA

6.7.3. Methods

6.7.3.1. Plasmid Construction

pVAX1-Nef^{mut}/Ag85B and pVAX1-Nef^{mut}/ESAT-6 were obtained by digesting the vector pVAX1-Nef^{mut}/E7^{OD}, where the E7^{OD} sequence was removed by digestion with Apa I. The Ag85B ORF and ESAT-6 ORF (GenScript) including both Kozak sequences and the ATG start codon at the 5′ end, were inserted at the Apa I site at the 3′ end in each plasmid in a way that the ligation with downstream heterologous sequences digested by Apa I resulted in a unique, in frame sequence.

Human embryonic kidney HEK 293 cells (American Type Culture Collection, CRL-1573) were grown in DMEM (w/glucose 4.5 g/l w/o L-Glutamine; Euroclone #ECB1501L) supplemented with 10% fetal bovine serum (FBS; Euroclone, #ECS0180L), L-Glutamine (4 mM, Euroclone, #ECB3000D) and penicillin/streptomycin (100 U/l, Euroclone, #ECB3001D).

3.5×10⁵ HEK 293 cells were seeded on 12 well plates. Cells were transfected with plasmid DNA 24 h after seeding, at a confluence of 80-90% with Lipofectamine LTX & Plus Reagent (LifeTechnologies, #A12621). 500 ng of plasmid DNA diluted in Opti-MEM (Gibco, #31985062) were transfected using 3.5 microliters of lipofectamine and 1 microliters of Plus Reagent.

6.7.3.2. Exosome Isolation

Cells and debris were removed from the culture medium with a first centrifugation for 30 min at 2.000 g. Total exosomes were isolated from culture medium with Total Exosome Isolation Reagent (LifeTechnologies, #4478359) according to manufacturer's instructions. In brief, 0.5 V of the reagent was added to the culture media samples and incubated overnight at 4° C. Exosomes were collected by centrifugation at 10.0000 g for 1 h at 4° C. The pelleted exosomes were lysed in Laemmli buffer (4% SDS, 16% glycerol, 40 mM Tris-HCl pH 6.8) supplemented with protease inhibitors (cOmplete™ and EDTA-free Protease Inhibitor cocktail; Roche, #11873580001).

6.7.3.3. Western Blot

Proteins were quantified with Pierce BCA Protein Assay Kit (LifeTechnologies, #23225) according to the manufacturer's instructions. Proteins were resolved with precast gels, 4-15% (Biorad, #4568084; #4568083) in Tris-Glycine buffer (3% Tris-base; 14.4%, Glycine, 1% SDS). Marker to visualize protein molecular weight was Precision Plus Protein Dual Color Standards (Biorad, #1610374). Semi-dry protein transfer on 0.2 m nitrocellulose membrane was performed with Trans-Blot Turbo (Biorad) 7 min at 2.5 A constant (up to 25 V). Blocking of the membrane was performed with 5% non-fat dry milk in TBST (500 mM Tris HCl pH 7.5, 500 mM NaCl, 0.15% Tween20). The following primary antibodies were used: anti-VINCULIN (1:5.000 Millipore, #MAB2081), anti-GFP 1:3.000-1:1.000 (Millipore, #MAB3580); anti-HIV1 NEF 1:5.000 (Abcam, #ab42358), anti-ALIX 1:500 (LifeTechnologies, #MA1-83977).

The following secondary antibody linked to horseradish peroxidase (Jackson ImmunoResearch) was used: anti-Mouse (1:5.000). Immunostained bands were detected using the chemiluminescent method (Euroclone, LiteAblot Plus/Extend/Turbo, #EMP011005/#EMP013001/#EMP013001) with ImageQuant LAS 500 instrument (GE HealthCare).

6.7.4. Results: Nef^{mut}-M. Tuberculosis Fusion Expression and Exosome Loading

FIGS. 24A and 24B show the detection of Nef^{mut}/Ag85B and Nef^{mut}/ESAT-6 fusion in transfected cells (cell lysates) and respective exosomes, with FIG. 24A showing the fusion protein expression in transfected cells and FIG. 24B showing the fusion protein in exosomes. Western blot analysis was performed with 30 μg of cell lysates from 293T cells transfected with DNA vectors expressing the indicated Nef^{mut}-based fusion products (lane 1 is Ag85B; lane 2 is ESAT-6), and with one fifth of volume of buffer where purified exosomes were resuspended after isolation of the respective supernatants. As control, cells transfected with EGFP (Clontech, PT-3027-5 6085-1) were included. Mouse monoclonal anti-Nef antibodies served to detect Nef^{mut}/Ag85B and Nef^{mut}/ESAT-6 product, while anti-Vinculin and anti-Alix were used as markers for cell lysates and exosomes, respectively. Molecular markers are provided in kDa.

### 6.8. Example 7: Nef^{mut} RNA Vaccines

FIG. 25 illustrates the two different approaches used to produce Nefmut/E7OD mRNA for use as RNA vaccines, with FIG. 25A illustrating use of linearized plasmid as template for in vitro transcription and FIG. 25B showing use of a PCR amplicon as a template for in vitro transcription.

As shown below, our experiments demonstrated that Nef^{mut}-E7^{OD} mRNA delivery is feasible in human cells. Nevertheless, the expression of the corresponding protein in cells transfected with the in vitro transcribed (IVT) mRNA was lower compared to the plasmid. However, our results showed that this is not due to a reduction of transfection efficiency. Probably, different improvements on the design of the transfected IVT mRNA (i.e. inclusion of 5′ and 3′ UTR able to stabilize the transcripts) could help to increase protein synthesis after mRNA transfection.

Notably, we have demonstrated that the NEF^{mut}-E7^{OD} protein expressed by a synthetic mRNA was uploaded in the exosomes after its transfection in human cells.

### 6.8.1. Example 7A: Transcription of Nef^{mut}/E7^{OD} Fusion from Linearized Plasmids and Expression in Transfected Cells

RNA was transcribed from 1 μg of linearized DNA plasmid with the T7 RNA polymerase of the mMESSAGE mMACHINE T7 Ultra Kit (Life Technologies, #AM1345) following the manufacturer's instructions. 10 μg of mRNA were transfected with Lipofectamine MessengerMAX (Life Technologies, LMNRNA001). Western blot analysis was performed with 30 μg of cell lysates from 293T cells transfected with in vitro transcribed mRNA expressing Nef^{mut}/E7^{OD} and 10 μg of buffer where purified exosomes were resuspended after isolation from culture medium with Total Exosome Isolation Reagent (Life Technologies, #4478359) according to manufacturer's instructions.

FIGS. 26A and 26B show the detection of Nef^{mut}/E7^{OD} fusion product in transfected cells and respective exosomes following mRNA delivery, with FIG. 26A showing the fusion protein expression in transfected cells and FIG. 26B showing the fusion protein in exosomes. As control, cells transfected with EGFP (Clontech, PT-3027-5 6085-1) were included. Mouse monoclonal anti-Nef antibodies served to detect Nef^{mut}/E7^{OD} product, while Vinculin and Alix were revealed as markers for cell lysates and exosomes, respectively. Relevant protein products are indicated by arrows. Molecular markers are provided in kDa.

### 6.8.2. Example 7B: Transcription of Nef^{mut}/E7^{OD} from PCR Fragments

6.8.2.1. Methods

6.8.2.1.1. Plasmid Construction

pVAX1-E7^{OD} was obtained by digesting the vector pVAX1-Nefmut/E7^{OD}, where the whole Nef^{mut}/E7^{OD} sequence was removed by digestion with EcoRI and Apa I. The E7^{OD} ORF (GenScript) including both Kozak sequences the ATG start codon at the 5′ end, was inserted between the EcoRI and Apa I sites.

6.8.2.1.2. Template Generation for In Vitro Transcription

200 ng of pEGFP-N1 (Clontech) was used as a substrate for PCR-amplification of the EGFP cDNA containing a T7 promoter sequence by using a high-fidelity Taq Polymerase (Platinum SuperFi Green DNA Polymerase) following manufacturer's instruction and these primers:

(SEQ ID NO: 80)
TAATACGACTCACTATAGGGCGCCACCATGGTGAG
and
TGCAGTGAAAAAAATGCTTTATTTG

(T7 promoter sequence is underlined) (SEQ ID NO: 81). The resulting PCR product was purified from agarose gel with the QIA quick Gel Extraction Kit (Qiagen, #28704) upon an electrophoretic separation to verify the correct size of the amplicon. The eluate was stored at −20° C. before the in vitro RNA transcription reaction.

pVax1-NEF^{mut}E7^{OD} and pVax1-E7^{OD} plasmids DNA were linearized through enzymatic digestion with SalI restriction enzyme (Promega #R605A) for 3 h at 37° C. The linearized vectors were then purified from agarose gel with QIAquick Gel Extraction Kit (Qiagen, #28704).

2 μg of plasmid DNA pVax1-NEF^{mut}E7^{OD} was digested with BcuI (SpeI, Thermo Scientific, (#ER1251) to cut away the T7 promoter sequence. The resulting fragment was gel purified and used as a template of a PCR reaction with a high-fidelity Taq Polymerase in order to generate DNA molecules containing only the E7 sequence; the following primers were used:

(SEQ ID NO: 82)
TAATACGACTCACTATAGGGCGCCACCATGCACGGC
and
(SEQ ID NO: 83)
GTCGACTTAGGGCTTCTGGCT

(giving rise to the fragment named short 3′ since finish just with the stop codon of the E7 protein) or with the same forward primer and a different reverse primer TGACACCTACTCAGACAATGCGATG (SEQ ID NO: 84) mapping on the cleavage site (CA) after the BGH polyA signal present in the vector (giving rise to the fragment called long 3′). The length of the PCR products was verified on agarose gel; amplicons were gel extracted by using the QIAquick Gel Extraction Kit. 200 ng of purified DNA templates were then used for the subsequent transcription reaction.

6.8.2.1.3. In Vitro RNA Transcription (IVT) and polyA Tailing

RNA was transcribed with the T7 RNA polymerase of the mMESSAGE mMACHINE T7 Ultra Kit (Life Technologies, #AM1345) following the manufacturer's instructions. In brief, 1 μg of linearized plasmid or 200 ng of purified PCR-products were used as a template in in vitro transcription reaction that incorporates a 5′ ARCA cap, carried out for 2 h at 37° C. and followed by treatment with TURBO DNase 15 min at 37° C. E-PAP enzyme was used 45 min at 37° C. for Poly(A) tailing reaction, stopped by the addition of 10 l of Ammonium Acetate Stop solution. In vitro transcribd (IVT) RNAs were extracted first with an equal volume of acidic phenol (Ambion, #AM9720) and then with chloroform, and, finally, precipitated with isopropanol. RNA was resuspended in nuclease-free water, quantified at nanophotometer (Implen), and stored at −80° C. The concentration of IVT RNAs varies from 150 to 1780 ng/l, with A260/280 near to 2, indicating pure RNA preparation. RNA was managed in a dedicated workplace with filter tips, RNAse-free plastic, and separated chemicals.

6.8.2.1.4. Analysis of Fusion Protein Expression

Human embryonic kidney HEK 293 cells (American Type Culture Collection, CRL-1573) were grown in DMEM (w/glucose 4.5 g/l w/o L-Glutamine; Euroclone #ECB1501L) supplemented with 10% fetal bovine serum (FBS; Euroclone, #ECS0180L), L-Glutamine (4 mM, Euroclone, #ECB3000D) and penicillin/streptomycin (100 U/l, Euroclone, #ECB3001D).

HEK293T cells were transiently transfected with the DNA vectors or in vitro transcribed RNAs described above. 2.5×10⁶ cells were seeded in 10 cm Petri dishes in 10% FCS DMEM. After 24 hours, cell transfections were carried out using 3 μg/ml polyethylenimine (PEI) (Sigma, cat no. 408727) and 5 μg DNA in 2% FCS DMEM.

2.5×10⁶ HEK 293 cells were seeded in P6 Petri dish and transfected with IVT mRNAs 24 h after seeding, at 80-90% of confluence with Lipofectamine MessengerMAX (Life Technologies, #LMNRNA001). For P6 plates, 10 μg of mRNAs in combination with 27 l of transfection reagent were used, diluted in Opti-MEM medium (Gibco, #31985060). Just before mRNA transfection, culture medium was replaced with 5% EV-depleted South American Fetal Bovine Serum (FBS). For extravesical depletion, FBS was centrifuged with Beckman LC40 Type 70 Ti Rotor for 4 h at 70,000×g. Culture medium or cells were collected at 18 h after transfection.

6.8.2.1.5. Exosome Isolation

Cells and debris were removed from the culture medium with a first centrifugation for 30 min at 2.000 g. Total exosomes were isolated from culture medium with Total Exosome Isolation Reagent (Life Technologies, #4478359) according to manufacturer's instructions. In brief, 0.5 V of the reagent was added to the culture media samples and incubated overnight at 4° C. Exosomes were collected by centrifugation at 10.0000 g for 1 h at 4° C. The pelleted exosomes were lysed in Laembli buffer (4% SDS, 16% glycerol, 40 mM Tris-HCl pH 6.8) supplemented with protease inhibitors (cOmplete™ and EDTA-free Protease Inhibitor cocktail; Roche, #11873580001).

6.8.2.1.6. Western Blot

Proteins were quantified with Pierce BCA Protein Assay Kit (Life Technologies, #23225) according to the manufacturer's instructions. Proteins were resolved with precast gels, 4-15% (Biorad, #4568084; #4568083) in Tris-Glycine buffer (3% Tris-base; 14.4%, Glycine, 1% SDS). Marker to visualize protein molecular weight was Precision Plus Protein Dual Color Standards (Biorad, #1610374). Semi-dry protein transfer on 0.2 m nitrocellulose membrane was performed with Trans-Blot Turbo (BioRad) 7 min at 2.5 A constant (up to 25 V). Blocking of the membrane was performed with 5% non-fat dry milk in TBST (500 mM Tris HCl pH 7.5, 500 mM NaCl, 0.15% Tween20). The following primary antibodies were used: anti-VINCULIN (1:5.000 Millipore, #MAB2081), anti-GFP 1:3.000-1:1.000 (Millipore, #MAB3580), anti-HPV16 E7 1:200 (NM Santa Cruz, sc-65711); anti-Human Papillomavirus 16 (E7), Abcam, #ab20191; anti-HIV1 NEF 1:5.000 (Abcam, #ab42358), anti-ALIX 1:500 (Life Technologies, #MA1-83977), anti-GAPDH 1:10.000 (Immunological Sciences, #MAB-10578).

The following secondary antibody linked to horseradish peroxidase (Jackson ImmunoResearch) was used: anti-Mouse (1:5.000). Immunostained bands were detected using the chemiluminescent method (Euroclone, LiteAblot Plus/Extend/Turbo, #EMP011005/#EMP013001/#EMP013001) with ImageQuant LAS 500 instrument (GE HealthCare).

6.8.2.2. Results

RNA was transcribed from 200 ng of purified DNA templates (PCR products) with the T7 RNA polymerase of the mMESSAGE mMACHINE T7 Ultra Kit (Life Technologies, #AM1345) following the manufacturer's instructions. 10 μg of mRNA were transfected with Lipofectamine MessengerMAX (Life Technologies, #LMNRNA001). Western blot analysis was performed with 30 μg of cell lysates from 293T cells transfected with in vitro transcribed mRNA expressing Nef^{mut}/E7^{OD} and 10 μg of buffer where purified exosomes were resuspended after isolation from culture medium with Total Exosome Isolation Reagent (Life Technologies, #4478359) according to manufacturer's instructions.

FIGS. 27A and 27B show the detection of Nefmut/E7OD fusion product by mRNA delivery in transfected cells and respective exosomes with FIG. XA showing the fusion protein expression in transfected cells and FIG. XB showing the fusion protein in exosomes. As control, cells transfected with E7^{OD} mRNA were included. Mouse monoclonal anti-Nef antibodies served to detect Nef^{mut}/E7^{OD} product, mouse monoclonal anti E7 served to detect E7^{OD} product while Vinculin and Alix were revealed as markers for cell lysates and exosomes, respectively. Relevant protein products are indicated by arrows in FIG. 27A and FIG. 27B. Molecular markers are provided in kDa.

### 7. EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

What is claimed is:

1. A fusion protein, comprising:
from N-terminus to C-terminus, (a) an exosome-anchoring polypeptide domain and (b) an immunogenic antigen polypeptide domain, optionally with a peptide linker therebetween,
wherein the exosome-anchoring polypeptide is a Nef^{mut} protein or a truncated Nef^{mut} protein having an amino acid sequence selected from SEQ ID NOs: 1-30.

2. The fusion protein of claim 1, wherein the exosome-anchoring polypeptide has the amino acid sequence of SEQ ID NO: 1.

3. The fusion protein of claim 1, wherein the exosome-anchoring polypeptide has the amino acid sequence of any one of SEQ ID NO:2-30.

4. The fusion protein of any one of claims 1 to 3, wherein the immunogenic antigen is a virus antigen, a bacterial antigen, or a tumor antigen.

5. The fusion protein of claim 4, wherein the immunogenic antigen is a virus antigen.

6. The fusion protein of claim 5, wherein the virus antigen is selected from the group consisting of: a human papillomavirus (HPV) antigen, a human immunodeficiency virus (HIV) antigen, a hepatitis B virus (HBV) antigen, a hepatitis C virus (HCV) antigen, an Ebola virus antigen, a West Nile virus antigen, a Crimean-Congo virus antigen, a dengue virus antigen, and an influenza virus antigen.

7. The fusion protein of claim 6, wherein the virus antigen is a human papillomavirus (HPV) antigen.

8. The fusion protein of claim 7, wherein the HPV antigen is E6 or E7 of human papillomavirus.

9. The fusion protein of claim 6, wherein the virus antigen is a human immunodeficiency virus (HIV) antigen.

10. The fusion protein of claim 9, wherein the HIV antigen is Gag or Tat of human immunodeficiency virus.

11. The fusion protein of claim 6, wherein the virus antigen is a hepatitis B virus (HBV) antigen.

12. The fusion protein of claim 11, wherein the HBV antigen is Core of hepatitis B virus.

13. The fusion protein of claim 6, wherein the virus antigen is a hepatitis C virus (HCV) antigen.

14. The fusion protein of claim 13, wherein the HCV antigen is Core, NS3, E1, or E2 of hepatitis C virus.

15. The fusion protein of claim 6, wherein the virus antigen is an Ebola virus antigen.

16. The fusion protein of claim 15, wherein the Ebola virus antigen is VP24, VP40, NP, or GP of Ebola virus.

17. The fusion protein of claim 6, wherein the virus antigen is a West Nile virus antigen.

18. The fusion protein of claim 17, wherein the West Nile virus antigen is NS3 of West Nile virus.

19. The fusion protein of claim 6, wherein the virus antigen is a Crimean-Congo virus antigen.

20. The fusion protein of claim 19, wherein the Crimean-Congo virus antigen is GP or NP of Crimean-Congo virus.

21. The fusion protein of claim 6, wherein the virus antigen is a dengue virus antigen.

22. The fusion protein of claim 6, wherein the virus antigen is an influenza virus antigen.

23. The fusion protein of claim 22, wherein the influenza virus is selected from the group consisting of: parainfluenza virus 1, parainfluenza virus 2, influenza A virus, and influenza B virus.

24. The fusion protein of claim 23, wherein the influenza virus is influenza A virus.

25. The fusion protein of claim 24, wherein the virus antigen is the nucleoprotein (NP) or the matrix protein (M1) of influenza A virus.

26. The fusion protein of claim 4, wherein the immunogenic antigen is a bacteria antigen.

27. The fusion protein of claim 26, wherein the bacteria antigen is a Mycobacterium tuberculosis antigen.

28. The fusion protein of claim 27, wherein the bacteria antigen is the antigen 85B (Ag85B) or the Early secretory antigenic target-6 (ESAT-6) of Mycobacterium tuberculosis.

29. The fusion protein of claim 4, wherein the immunogenic antigen is a parasite antigen.

30. The fusion protein of claim 29, wherein the parasite antigen is a Plasmodium antigen.

31. The fusion protein of claim 4, wherein the immunogenic antigen is a tumor antigen.

32. The fusion protein of claim 31, wherein the tumor antigen is a tumor-specific antigen.

33. The fusion protein of claim 31, wherein the tumor antigen is a tumor-associated antigen.

34. A polynucleotide encoding the fusion protein of any of the preceding claims.

35. The polynucleotide of claim 34, wherein the polynucleotide is DNA.

36. The polynucleotide of claim 34, wherein the polynucleotide is RNA.

37. The polynucleotide of claim 26, wherein the RNA is a messenger RNA (mRNA).

38. The polynucleotide of claim 37, wherein the mRNA is suitable for translation obtained by T7 RNA polymerase transcription from a DNA template.

39. A vector comprising at least one polynucleotide of any one of claims 34 to 38, wherein the vector expresses at least one fusion protein of any one of claims 1 to 33.

40. The vector of claim 39, wherein the vector is a plasmid vector.

41. The vector of claim 39, wherein the vector is a viral vector.

42. The vector of claim 41, wherein the viral vector is an adenovirus vector, an adeno-associated virus (AAV) vector, or a vaccinia vector.

43. An extracellular vesicle comprising the fusion protein, the polynucleotide, or the vector of any of the preceding claims.

44. The extracellular vesicle of claim 43, wherein the extracellular vesicle is an exosome.

45. A nanoparticle comprising the fusion protein, the polynucleotide, or the vector of any one of claims 1 to 42.

46. A vaccine composition comprising the fusion protein, the polynucleotide, the vector, the extracellular vesicle, or the nanoparticle of any of the preceding claims, and a pharmaceutically acceptable excipient.

47. The vaccine composition of claim 46, formulated for intramuscular administration.

48. A method of immunizing a subject against a desired antigen, comprising:
administering to the subject an effective amount of the fusion protein, the polynucleotide, the vector, the extracellular vesicle, the nanoparticle, or the pharmaceutical composition of any of the preceding claims,
wherein the immunogenic antigen polypeptide domain of the fusion protein, encoded by the polynucleotide, expressed by the vector, or included in the extracellular vesicle, nanoparticle, or pharmaceutical composition comprises the desired antigen.

49. The method of claim 48, wherein a plurality of immunogenic antigens are administered as fusions to the subject.

50. The method of claim 49, wherein the immunogenic antigens are expressed from the same vector or mRNA.

51. The method of claim 49, wherein the immunogenic antigens are expressed from different vectors or mRNAs.

52. The method of any one of claims 49 to 51, wherein the immunogenic antigens are administered simultaneously to the subject.

53. The method of any one of claims 48 to 52, wherein the disease or condition is a viral infection.

54. The method of any one of claims 48 to 52, wherein the disease or condition is cancer.

55. The method of any one of claims 48 to 54, wherein the fusion protein, the polynucleotide, the vector, the extracellular vesicle, the nanoparticle, or the pharmaceutical composition is administered by intramuscular administration.

56. The method of claim 55, wherein the method further comprises electroporation immediately after the intramuscular administration.

57. A method of inducing an antigen-specific cytotoxic T-lymphocyte (CTL) and/or antigen-specific CD4⁺ T cell response in a subject in need thereof, comprising:
administering to the subject an effective amount of the fusion protein, the polynucleotide, the vector, the extracellular vesicle, the nanoparticle, or the pharmaceutical composition of any one of claims 1 to 47.

58. The method of claim 57, wherein a plurality immunogenic antigens fused to an exosome-anchoring polypeptide domain are administered to the subject.

59. The method of claim 58, wherein the immunogenic antigens are expressed from the same vector or mRNA.

60. The method of claim 58, wherein the immunogenic antigens are expressed from different vectors or mRNAs.

61. The method of any one of claims 58 to 60, wherein the immunogenic antigens are administered simultaneously to the subject.

62. The method of any one of claims 57 to 61, wherein the subject has or is at risk for a viral infection.

63. The method of any one of claims 57 to 61, wherein the subject has or is at risk for cancer.

64. The method of any one of claims 57 to 61, wherein the subject has or is at risk for a bacterial infection.

65. The method of any one of claims 57 to 63, wherein the fusion protein, the polynucleotide, the vector, the extracellular vesicle, the nanoparticle, or the pharmaceutical composition is administered by intramuscular administration.

66. The method of claim 65, wherein the method further comprises electroporation immediately after the intramuscular administration.

67. A fusion protein, comprising:
from N-terminus to C-terminus, (a) an exosome-anchoring polypeptide domain and (b) an immunogenic antigen polypeptide domain,
wherein the exosome-anchoring polypeptide is a Nef^{mut} protein or a truncated Nef^{mut} protein having an amino acid sequence selected from SEQ ID NOs: 1-30, and
wherein the immunogenic antigen has the amino acid sequence of SEQ ID NO: 33, SEQ ID NO: 36, SEQ ID NO: 51 or SEQ ID NO: 75.

68. The fusion protein of claim 67, wherein the exosome-anchoring polypeptide is a Nef^{mut} protein having the amino acid sequence of SEQ ID NO: 1, and wherein the immunogenic antigen has the amino acid sequence of SEQ ID NO: 33.

69. The fusion protein of claim 68, wherein the fusion protein has the amino acid sequence of SEQ ID NO: 39.

70. The fusion protein of claim 67, wherein the exosome-anchoring polypeptide is a Nef^{mut} protein having the amino acid sequence of SEQ ID NO: 1, and wherein the immunogenic antigen has the amino acid sequence of SEQ ID NO: 36.

71. The fusion protein of claim 70, wherein the fusion protein has the amino acid sequence of SEQ ID NO: 42.

72. The fusion protein of claim 67, wherein the exosome-anchoring polypeptide is a truncated Nef^{mut} protein having the amino acid sequence of SEQ ID NO: 30, and wherein the immunogenic antigen has the amino acid sequence of SEQ ID NO: 33.

73. The fusion protein of claim 72, wherein the fusion protein has the amino acid sequence of SEQ ID NO: 45.

74. The fusion protein of claim 67, wherein the exosome-anchoring polypeptide is a truncated Nef^{mut} protein having the amino acid sequence of SEQ ID NO: 30, and wherein the immunogenic antigen has the amino acid sequence of SEQ ID NO: 36.

75. The fusion protein of claim 74, wherein the fusion protein has the amino acid sequence of SEQ ID NO: 48.

76. The fusion protein of claim 67, wherein the exosome-anchoring polypeptide is a Nef^{mut} protein having the amino acid sequence of SEQ ID NO: 30, and wherein the immunogenic antigen has the amino acid sequence of SEQ ID NO: 51.

77. The fusion protein of claim 76, wherein the fusion protein has the amino acid sequence of SEQ ID NO: 85.

78. The fusion protein of claim 67, wherein the exosome-anchoring polypeptide is a Nef^{mut} protein having the amino acid sequence of SEQ ID NO: 30, and wherein the immunogenic antigen has the amino acid sequence of SEQ ID NO: 75.

79. The fusion protein of claim 68, wherein the fusion protein has the amino acid sequence of SEQ ID NO: 87.

80. A polynucleotide encoding the fusion protein of any one of claims 67 to 79.

81. The polynucleotide of claim 80, wherein the polynucleotide is DNA.

82. The polynucleotide of claim 81, having the nucleotide sequence of SEQ ID NOs: 38, 41, 44, 47, 74 or 79.

83. The polynucleotide of claim 80, wherein the polynucleotide is RNA.

84. A vector comprising at least one polynucleotide of any one of claims 80 to 83, wherein the vector expresses at least one fusion protein of any one of claims 67 to 75.

85. The vector of claim 84, wherein the vector is a plasmid vector.

86. The vector of claim 84, wherein the vector is a viral vector.

87. An extracellular vesicle comprising the fusion protein, the polynucleotide, or the vector of any one of claims 67 to 86.

88. The extracellular vesicle of claim 87, wherein the extracellular vesicle is an exosome.

89. A nanoparticle comprising the fusion protein, the polynucleotide, or the vector of any one of claims 67 to 86.

90. A pharmaceutical composition comprising the fusion protein, the polynucleotide, the vector, the extracellular vesicle, or the nanoparticle of any one of claims 67 to 89, and a pharmaceutically acceptable excipient.

91. The pharmaceutical composition of claim 90, formulated for intramuscular administration.

92. A method of treating or preventing a human papillomavirus (HPV) infection, comprising:
administering to a patient who has or is at risk for HPV infection an effective amount of the fusion protein, the polynucleotide, the vector, the extracellular vesicle, the nanoparticle, or the pharmaceutical composition of any one of claims 67 to 91.

93. The method of claim 92, wherein E6 and E7 antigens of HPV16 are administered to the patient.

94. The method of claim 93, wherein the E6 and E7 antigens of HPV16 are expressed from the same vector or mRNA.

95. The method of claim 93, wherein the E6 and E7 antigens of HPV16 are expressed from different vectors or mRNAs.

96. The method any one of claims 93 to 95, wherein the E6 and E7 antigens of HPV16 are administered simultaneously to the patient.

97. The method of any one of claims 92 to 96, wherein the fusion protein, the polynucleotide, the vector, the extracellular vesicle, the nanoparticle, or the pharmaceutical composition is administered by intramuscular administration.

98. The method of claim 97, wherein the method further comprises electroporation immediately after the intramuscular administration.

99. A method of inducing a cytotoxic T-lymphocyte (CTL) and/or antigen-specific CD4⁺ T cell response in a patient who has or is at risk for HPV infection, comprising:
administering to a patient who has or is at risk for HPV infection an effective amount of the fusion protein, the polynucleotide, the vector, the extracellular vesicle, the nanoparticle, or the pharmaceutical composition of any one of claims 67 to 91.

100. The method of claim 99, wherein E6 and E7 antigens of HPV16 are administered to the patient.

101. The method of claim 100, wherein the E6 and E7 antigens of HPV16 are expressed from the same vector or mRNA.

102. The method of claim 100, wherein the E6 and E7 antigens of HPV16 are expressed from different vectors or mRNAs.

103. The method any one of claims 100 to 102, wherein the E6 and E7 antigens of HPV16 are administered simultaneously to the patient.

104. The method of any one of claims 99 to 103, wherein the fusion protein, the polynucleotide, the vector, the extracellular vesicle, the nanoparticle, or the pharmaceutical composition is administered by intramuscular administration.

105. The method of claim 104, wherein the method further comprises electroporation immediately after the intramuscular administration.
